(19) United States
(12) Patent Application Publication
(10) Pub. No.: US 2004/0072354 A1
(43) Pub. Date: Apr. 15, 2004

(54) Natural promoters for gene expression and metabolic monitoring in bacillus species

(76) Inventors: Laura A. Bedzyk, Odessa, DE (US); Tao Wang, Hockessin, DE (US); Rick W. Ye, Hockessin, DE (US)

Correspondence Address: S Neil Feltham, E I du Pont de Nemours And Company, Legal - Patents, Wilmington, DE, 19898, US

(21) Appl. No.: 10/275,191

(22) PCT Filed: Jun. 29, 2001
(86) PCT No.: PCT/US01/20873

(51) Int. Cl. C12N 15/74; C12N 1/21

(52) U.S. Cl. 435/471; 435/252.31; 435/320.1

(57) ABSTRACT

Genes have been identified in the Bacillus genome that are responsive to various metabolic conditions and growth cycle changes. The new responsiveness of these genes allows for their use in regulated gene expression in Bacillus sp. and for the monitoring of bioreactor health.

### FIELD OF THE INVENTION

This application claims the benefit of U.S. Provisional Application No. 60/214,967, filed Jun. 29, 2000 and of U.S. Provisional Application No. 60/268,320, filed Feb. 13, 2001.

This invention is in the field of bacterial gene expression and fermentation monitoring. More specifically, the invention relates to the use of promoter regions isolated from a Bacillus sp. for regulated gene expression and process control monitoring of fermentation cultures.

### BACKGROUND INFORMATION

The Bacillus bacteria are useful.production hosts for a variety of biological materials including enzymes, antibiotics and other pharmaceutically active products. The use of Bacillus species for production of biomaterials is particularly advantageous as compared with other microbial production hosts, particularly gram negative organisms. For example, the most common gram negative organism used in industrial microbiology, E. coli, suffers from the presence of endotoxins which, being pathogenic in man, are undesirable products. Additionally, gram negative hosts often produce proteins in inactive or insoluble forms which necessitate expensive reactivation and purification schemes. In contrast, Bacillus has a highly develop secretory system for the expression and transport of active proteins to the growth medium, thereby facilitating purification and eliminating costly reactivation procedures. Thus Bacillus is a production host of choice for many industrial applications. Methods to enhance gene expression or monitor culture health and biomass production for these organisms are desirable.

The Bacillus sp. and particularly Bacillus subtilis is well-known for its stationary metabolism (Stragier, P. and Losick, R. 1996. Annu. Rev. Genet. 30:297-341, Lazazzera, B. A. 2000. Curr. Opin Microbiol.3:177-182, Msadek, T. 1999. Trends Microbiol. 7:201-207). A wide variety of genes, such as those involved in catabolism, amino acid biosynthesis, antibiotic production, cell to cell communication, competence, and sporulation, are induced at stationary phase. Bacillus subtilis is also a facultative bacterium capable of growing in the presence or absence of oxygen. In the absence of oxygen, Bacillus subtilis uses nitrate or nitrite as the alternative electron acceptor or grows in the presence of pyruvate (Nakano et al., 1998. Annu. Rev. Microbiol. 52:165-190). It has been shown that promoters that control the expression of genes involved in nitrate and nitrite respiration are under the control of the two-component signal transduction system ResDE (Sun et al., 1996. J. Bacteriol. 178:1374-1385).

In general, prokaryotic promoters can play an important role in biotechnology particularly in expressing those genes whose products can be made in their active forms and in large quantities in prokaryotic hosts. Identification of the promoters regulated during stationary phase growth when the cells reach a certain density is valuable when Bacillus subtilis is used as a production host. Similarly, promoters induced by oxygen-limiting conditions are very applicable in industrial settings since oxygen level can adjusted easily.

Investigation of promoter activity in Bacillus subtilis or any other bacterium often employs Northern or Southern blots, enzymatic assays, or reporting genes. These methods permit monitoring of the effect of environmental changes on gene expression by comparing expression levels of a limited number of genes. Furthermore, they often enable investigation of one or a subset of the physiological events and fail to monitor the comprehensive responses of a preponderance of individual genes in the genome of an organism in reliable and useful manner.

With the advances in genomic research, a powerful way to identify promoters is the use of DNA microarray. DNA microarray is a technology used to explore gene expression profiles in a genome-wide scale (DeRisi, J. L., V. R. Iyer, and P. O. Brown. 1997. Science. 278:680-686). It allows for the identification of genes that are expressed in different growth stages or environmental conditions. This is especially valuable for industrial environments where the conditions for promoter induction have to be convenient, cost effective and compatible with a specific bio-manufacturing process. A significant advance in the art would be a process which would allow for analysis of the timing and extent of induction of most of the genes involved in production and provide inclusive information on the state of the biomass and cell response to growth conditions.

The problem to be solved therefore is to identify genes within the Bacillus genome that are regulated by metabolic conditions or growth cycle changes, and to apply these genes for gene expression and bioreactor monitoring in Bacillus sp. cultures. Applicants have solved the stated problem by using microarray technology to identify genes which are responsive to oxygen depletion, the presence of nitrite, or are sensitive to various stages of the stationary growth phase.

### SUMMARY OF THE INVENTION

The present invention provides a method for the expression of a coding region of interest in a Bacillus sp comprising: a) providing a transformed Bacillus sp cell containing a chimeric gene comprising a nucleic acid fragment consisting of the promoter region of a Bacillus gene operably linked to a coding region of interest expressible in a Bacillus sp, wherein the nucleic acid fragment comprising the promoter region of a Bacillus gene is selected from the group consisting of narGHJI, csn, yncm, yncD, yvaWXY, ydjL, sunA, and yolIJK and homologues thereof; and b) growing the transformed Bacillus sp cell of step (a) in the absence of oxygen wherein the chimeric gene of step (a) is expressed.

Optionally cells may be grown in the presence of oxygen to increase the cell biomass and the oxygen level then decreased to allow for induction and expression for the chimeric gene. Subsequently oxygen levels may be restored to permit bioconversion utilizing the product of the expressed coding region.

Similarly the invention provides a method for the expression of a coding region of interest in a Bacillus sp comprising: a) providing a transformed Bacillus sp cell containing a chimeric gene comprising a nucleic acid fragment consisting of the promoter region of a Bacillus gene operably linked to a coding region of interest expressible in a Bacillus sp, wherein the nucleic acid fragment comprising the promoter region of a Bacillus gene is selected from the group consisting of feuABC, ykuNOP, and dhbABC, and homologues thereof; and b) growing the transformed Bacillus sp cell of step (a).in the absence of oxygen and in the presence of nitrite wherein the chimeric gene of step (a) is expressed.

In another embodiment the invention provides a method for the expression of a coding region of interest in a Bacillus sp comprising: a) providing a transformed Bacillus sp cell containing a chimeric gene comprising a nucleic acid fragment consisting of the promoter region of a Bacillus gene operably linked to a coding region of interest expressible in a Bacillus sp, wherein the nucleic acid fragment comprising the promoter region of a Bacillus gene is selected from the group consisting of ycgAN, dhaS rapF, rapG, rapH, rapK, yqhIJ, yveKLMNOPQST, yhfRSTUV, csn, yncM, yvyD, yvaWXY, ydjL, sunA, and yolIJK, and homologues thereof; and b) growing the transformed Bacillus sp cell of step (a) in the presence of oxygen until the cell reaches about T0 of the stationary phasewherein the chimeric gene of step (a) is expressed.

In an alternate embodiment the invention provides a method for the expression of a coding region of interest in a Bacillus sp comprising: a) providing a transformed Bacillus sp cell containing a chimeric gene comprising a nucleic acid fragment consisting of the promoter region of a Bacillus gene operably linked to a coding region of interest expressible in a Bacillus sp, wherein the nucleic acid fragment comprising the promoter region of a Bacillus gene is selected from the group consisting of acoABCL, and glvAC, and homologues thereof; and b) growing the transformed Bacillus sp cell of step (a) in the presence of oxygen until the cell reaches about T1 of the stationary phasewherein the chimeric gene of step (a) is expressed.

In yet another embodiment the invention provides a method for the expression of a coding region of interest in a Bacillus sp comprising: a) providing a transformed Bacillus sp cell containing a chimeric gene comprse a nucleic acid fragment consisting of the promoter region of a Bacillus gene operably linked to a coding region of interest expressible in a Bacillus sp, wherein the nucleic acid fragment comprising the promoter region of a Bacillus gene is selected from the group consisting of yxjCDEF, yngEFGHI, yjmCDEFG, ykfABCD, and yodOPRST; and homologues thereof; ahd b) growing the transformed Bacillus sp cell of step (a) in the presence of oxygen until the cell reaches about T3 of the stationary phase wherein the chimeric gene of step (a) is expressed.

Within the context of the present invention the Bacillus sp. cell is selected from the species consisting of Bacillus subtillus, Bacillus thuringiensis, Bacillus anthiacis, Bacillus cereus, Bacillus brevis, Bacillus megaterium, Bacillus intermedius, Bacillus thermoamyloliquefaciens, Bacillus arnyloliquefaciens, Bacillus circulans, Bacillus lichenifornis, Bacillus macerans, Bacillus sphaericus, Bacillus stearothernophilus, Bacillus laterosporus, Bacillus acidocaldarius, Bacillus pumilus, and Bacillus pseudofirnus.

Additionally within the context of the present invention the coding region of interest is selected from the group consisting of crtE crtB, pds, crtD, crtL, crtZ, crtX crtO, phaC, phaE, efe, pdc, adh, genes encoding limonene synthase, pinene synthase, bornyl synthase, phellandrene synthase, cineole synthase, sabinene synthase, and taxadiene synthase.

Additionally the present invention provides a method for monitoring the state of the cell metabolism of a Bacillus sp. culture comprising: a) providing a culture of actively growing Bacillus sp. cells; and b) measuring the expression levels of a pool of genes isolated from the Bacillus cells of step (a), the pool of genes comprising narGHJI, feuABC, ykuNOP, dhbABC, ydjL, sunA, yolIJK, csn ,yncM, yvyD, yvaWXY, yhfRSTUV, yveKLMNOPQST, dhaS, rapF, rapG, rapH, rapK, ycgMN, yqhIJ, glvAC, acoABCL, yxjCDEF, yngEFGHI yjmCDEFG, ykfABCD, yodOPRST, alsT, and yxeKLMN, and homologues thereof.

In a preferred embodiment the invention provides a monitoring method wherein an actively growing culture is grown in the absence of oxygen and the expression of genes narGHJI, ydjL, sunA, yolIJK, csn, yncM, yvyD, and yvaWXY are up-regulated in the log phase.

In another preferred embodiment the invention provides a monitoring method wherein the actively growing culture is grown in the absence of oxygen and in the presenece of nitrite and the expression of genes feuABC, ykuNOP, and dhbABC are up-regulated in the log phase.

Similarly the invention provides a monitoring method wherein the expression of genes narGHJI is down-regillated at about T0 of the stationary phase.

Additionally the invention provides a monitoring method wherein the actively growing culture is grown in the presence of oxygen and the expression of genes ycgMN, yqhIJ, ydjL, sunA, yolIJK, csn, yncM, yvyD, yvaWXY, yhfRSTUV, yveKLAMOPQST, dhaS, rapF, rapG, rapH, rapK are up-regulated at about T0 of the stationary phase.

Similarly the invention provides a monitoring method wherein the actively growing culture is grown in the presence of oxygen and the expression of genes, acoABCL and glvAC are up-regulated at about T1 of the stationary phase.

In an alternate embodiment the invention provides a monitoring method wherein the actively growing culture is grown in the presence of oxygen and the expression of genes, yxjCDEF, yngEFGHI yjmCDEFG, ykfABCD, and yodOPRST are up-regulated at about T3 of the stationary phase.

In another embodiment the invention provides a monitoring method wherein the actively growing culture is grown in the presence of oxygen and the expression of genes, alsT and yxeKLAN are down-regulated at stationary phase or under nutrient-limiting conditions.

### BRIEF DESCRIPTION OF THE SEQUENCES

The invention can be more fully understood from the following detailed description and the accompanying sequence descriptions which form a part of this application.

The following sequences conform with 37 C.F.R. 1.821-1.825 (“Requirements for Patent Applications Containing Nucleotide Sequences and/or Amino Acid Sequence Disclosures—the Sequence Rules”) and are consistent with World Intellectual Property Organization (WIPO) Standard ST.25 (1998) and the sequence listing requirements of the EPO and PCT (Rules 5.2 and 49.5(a-bis), and Section 208 and Annex C of the Administrative Instructions). The symbols and format used for nucleotide and amino acid sequence data comply with the rules set forth in 37 C.F.R. §1.822.

| | SEQ ID | | SEQ ID |
|---|---|---|---|
| Description | Nucleic acid | Description | Nucleic acid |
| Nucleotide sequence | 1 | Nucleotide sequence | 42 |
| of a narG gene | | of a acoB gene | |
| Nucleotide sequence | 2 | Nucleotide sequence | 43 |
| of a narH gene | | of a acoC gene | |
| Nucleotide sequence | 3 | Nucleotide sequence | 44 |
| of a narJ gene | | of a acoL gene | |
| Nucleotide sequence | 4 | Nucleotide sequence | 45 |
| of a narI gene | | of a yhfR gene | |
| Nucleotide sequence | 5 | Nucleotide sequence | 46 |
| of a csn gene | | of a yhfS gene | |
| Nucleotide sequence | 6 | Nucleotide sequence | 47 |
| of a yncM gene | | of a yhfT gene | |
| Nucleotide sequence | 7 | Nucleotide sequence | 48 |
| of a yvyD gene | | of a yhfU gene | |
| Nucleotide sequence | 8 | Nucleotide sequence | 49 |
| of a yvaW gene | | of a yhfV gene | |
| Nucleotide sequence | 9 | Nucleotide sequence | 50 |
| of a yvaX gene | | of a glvA gene | |
| Nucleotide sequence | 10 | Nucleotide sequence | 51 |
| of a yvaY gene | | of a glvC gene | |
| Nucleotide sequence | 11 | Nucleotide sequence | 52 |
| of a ydjL gene | | of a yxjC gene | |
| Nucleotide sequence | 12 | Nucleotide sequence | 53 |
| of a sunA gene | | of a yxjD gene | |
| Nucleotide sequence | 13 | Nucleotide sequence | 54 |
| of a yolI gene | | of a yxjE gene | |
| Nucleotide sequence | 14 | Nucleotide sequence | 55 |
| of a yolJ gene | | of a yxjF gene | |
| Nucleotide sequence | 15 | Nucleotide sequence | 56 |
| of a yolK gene | | of a yngE gene | |
| Nucleotide sequence | 16 | Nucleotide sequence | 57 |
| of a feuA gene | | of a yngF gene | |
| Nucleotide sequence | 17 | Nucleotide sequence | 58 |
| of a feuB gene | | of a yngG gene | |
| Nucleotide sequence | 18 | Nucleotide sequence | 59 |
| of a feuC gene | | of a yngH gene | |
| Nucleotide sequence | 19 | Nucleotide sequence | 60 |
| of a ykuN gene | | of a yngI gene | |
| Nucleotide sequence | 20 | Nucleotide sequence | 61 |
| of a ykuO gene | | of a yjmC gene | |
| Nucleotide sequence | 21 | Nucleotide sequence | 62 |
| of a ykuP gene | | of a yjmD gene | |
| Nucleotide sequence | 22 | Nucleotide sequence | 63 |
| of a dhbA gene | | of a yjmE gene | |
| Nucleotide sequence | 23 | Nucleotide sequence | 64 |
| of a dhbB gene | | of a yjmF gene | |
| Nucleotide sequence | 24 | Nucleotide sequence | 65 |
| of a dhbC gene | | of a yjmG gene | |
| Nucleotide sequence | 25 | Nucleotide sequence | 66 |
| of a dhaS gene | | of a ykfA gene | |
| Nucleotide sequence | 26 | Nucleotide sequence | 67 |
| of a rapF gene | | of a ykfB gene | |
| Nucleotide sequence | 27 | Nucleotide sequence | 68 |
| of a rapG gene | | of a ykfC gene | |
| Nucleotide sequence | 28 | Nucleotide sequence | 69 |
| of a rapH gene | | of a ykfD gene | |
| Nucleotide sequence | 29 | Nucleotide sequence | 70 |
| of a rapK gene | | of a yodO gene | |
| Nucleotide sequence | 30 | Nucleotide sequence | 71 |
| of a yqhI gene | | of a yodP gene | |
| Nucleotide sequence | 31 | Nucleotide sequence | 72 |
| of a yqhJ gene | | of a yodR gene | |
| Nucleotide sequence | 32 | Nucleotide sequence | 73 |
| of a yveK gene | | of a yodS gene | |
| Nucleotide sequence | 33 | Nucleotide sequence | 74 |
| of a yveL gene | | of a yodT gene | |
| Nucleotide sequence | 34 | Nucleotide sequence | 75 |
| of a yveM gene | | of a ycgM gene | |
| Nucleotide sequence | 35 | Nucleotide sequence | 76 |
| of a yveN gene | | of a ycgN gene | |
| Nucleotide sequence | 36 | Nucleotide sequence | 77 |
| of a yveO gene | | of a alsT gene | |
| Nucleotide sequence | 37 | Nucleotide sequence | 78 |
| of a yveP gene | | of a yxeN gene | |
| Nucleotide sequence | 38 | Nucleotide sequence | 79 |
| of a yveQ gene | | of a yxeM gene | |
| Nucleotide sequence | 39 | Nucleotide sequence | 80 |
| of a yveS gene | | of a yxeL gene | |
| Nucleotide sequence | 40 | Nucleotide sequence | 81 |
| of a yveT gene | | of a yxeK gene | |
| Nucleotide sequence | 41 | | |
| of a acoA gene | | | |

### DETAILED DESCRIPTION OF THE INVENTION

The present invention advances the art by providing:

(i) the first instance of a comprehensive survey of endogenous promoters and metabolic markers with a micro-array comprising greater than 75% of all open reading frames from a Bacillus subtilis, overcoming the problems of high concentration of endogenous RNAase and ribosomal RNA;

(ii) A method for the expression of a coding region of interest in a Bacillus sp during the anaerobic growth or induced by oxygen-limiting conditions.

(iii) A method for the expression of a coding region of interest in a Bacillus sp during the stationary growth phase.

(iv) A method for monitoring the metabolic state of Bacillus sp with gene expression patterns generated by DNA microarray.

The present invention has utility in many different fields. Gene expression profiles can be used to detect genotypic alterations among strains. The present invention enables the monitoring of expression profiles when changes in growth conditions occur. The genes of the present invention may be used in a modeling system to test perturbations in fermentation process conditions which will determine the requirements for the high yield of bioprocess production. Additionally, many discovery compounds can be screened by comparing a gene expression profile to a known compound that affects the desirable target gene products.

In this disclosure, a number of terms and abbreviations are used. The following definitions are provided.

A “nucleic acid” is a polymeric compound comprised of covalently linked subunits called nucleotides. Nucleic acid includes polyribonucleic acid (RNA) and polydeoxyribonucleic acid (DNA), both of which may be single-stranded or double-stranded. DNA includes cDNA, genomic DNA, synthetic DNA, and semi-synthetic DNA.

As used herein, an “isolated nucleic acid fragment” is a polymer of RNA or DNA that is single- or double-stranded, optionally containing synthetic, non-natural or altered nucleotide bases. An isolated nucleic acid fragment in the form of a polymer of DNA may be comprised of one or more segments of cDNA, genomic DNA or synthetic DNA.

A nucleic acid fragment is “hybridizable” to another nucleic acid fragment, such as a cDNA, genomic DNA, or RNA, when a single stranded form of the nucleic acid fragment can anneal to the other nucleic acid fragment under the appropriate conditions of temperature and solution ionic strength. Hybridization and washing conditions are well known and exemplified in Sambroolc, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein (entirely incorporated herein by reference). The conditions of temperature and ionic strength determine the “stringency” of the hybridization. Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions. One set of preferred conditions uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. A more preferred set of stringent conditions uses higher temperatures in which the washes are identical to those above except for the temperature of the final two 30 min washes in 0.2×SSC, 0.5% SDS was increased to 60° C. Another preferred set of highly stringent conditions uses two final washes in 0.1×SSC, 0.1% SDS at 65° C. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tm for hybrids of nucleic acids having those sequences. The relative stability (corresponding to higher Tm) of nucleic acid hybridizations decreases in the following order: RNA:RNA, DNA:RNA, DNA:DNA. For hybrids of greater than 100 nucleotides in length, equations for calculating Tm have been derived (see Sambrook et al., supra, 9.50-9.51). For hybridizations with shorter nucleic acids, i.e., oligonucleotides, the position of mismatches becomes more important, and the length of the oligonucleotide determines its specificity (see Sambrook et al., supra, 11.7-11.8). In one embodiment the length for a hybridizable nucleic acid is at least about 10 nucleotides. Preferable a minimum length for a hybridizable nucleic acid is at least about 15 nucleotides; more preferably at least about 20 nucleotides; and most preferably the length is at least 30 nucleotides. Furthermore, the slilled artisan will recognize that the temperature and wash solution salt concentration may be adjusted as necessary according to factors such as length of the probe.

As used herein, the term “oligonucleotide” refers to a nucleic acid, generally of at least 18 nucleotides, that is hybridizable to a genomic DNA molecule, a cDNA molecule, or an mRNA molecule. Oligonucleotides can be labeled, e.g., with ³²P-nucleotides or nucleotides to which a label, such as biotin, has been covalently conjugated. In one embodiment, a labeled oligonucleotide can be used as a probe to detect the presence of a nucleic acid according to the invention. In another embodiment, oligonucleotides (one or both of which may be labeled) can be used as PCR primers, either for cloning fall length or a fragment of a nucleic acid of the invention, or to detect the presence of nucleic acids according to the invention. In a further embodiment, an oligonucleotide of the invention can form a triple helix with a DNTA molecule. Generally, oligonucleotides are prepared synthetically, preferably on a nucleic aid synthesizer. Accordingly, oligonucleotides can be prepared with non-naturally occurring phosphoester analog bonds, such as thioester bonds, etc.

A “gene” refers to an assembly of nucleotides that encode a polypeptide, and includes cDNA and genomic DNA nucleic acids. “Gene” also refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. Chimeric genes of the present invention will typically comprise an inducible promoter operably linked to a coding region of interest. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

The term “inducible gene” means any Bacillus gene whose expression is up-regulated in response to a specific stress or stimulus. Inducible genes of the present invention include the genes identified as narGHJI, feuABC, ykuNOP, dhbABC, ydjL, sunA, yolIJK, csn, yncM, yvyD, yvaWXY, yhfRSTUV, yveKLMNOPQST, dhaS, rapF, rapG, rapH, rapK, yqhIJ, ycgMN, glvAC, acoABCL,yxjCDEF, yngEFGHI, yjmCDEFG, ykfABCD, yodOPRST, alsT, and yxeKLMN.

“Coding sequence” or “open reading frame” (ORF) refers to a DNA sequence that codes for a specific amino acid sequence. A coding sequence is “under the control” of transcriptional and translational control sequences in a cell when RNA polymerase transcribes the coding sequence into mRNA, which is then trans-RNA spliced (if the coding sequence contains introns) and translated into the protein encoded by the coding sequence. The term “coding region of interest” refers to any coding region or open reading frame that is expressible in a desired host and may be regulated by the promoter of the present inducible genes.

“Promoter” refers to a DNA sequence capable of controlling the expression of a coding sequence or functional RNA. In general, a coding sequence is located 3′ to a promoter sequence Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity. “Inducible promoter” mean any promoter that is responsive to a particular stimulus. Inducible promoters of the present invention will typically be derived from the “inducible genes” and will be responsive to various metabolic conditions (oxygen input, nutrient composition, environmental stress such as pH and temperature changes, or overproduction of a particular product or expression of a foreign gene. product) or stages in the cell growth cycle.

The term “expression”, as used herein, refers to the transcription and stable accumulation of sense (mRNA) or antisense RNA derived from the nucleic acid fragment of the invention. Expression may also refer to translation of mRNA into a polypeptide.

The term “up-regulated” as applied to gene expression means the mRNA transcriptional level of a particular gene or region in the test condition is increased as compared to the control condition.

The term “down-regulated” as applied to gene expression means the mRNA transcriptional level of a particular gene or region in the test condition is decreased as compared to the control condition.

The term “homologue” as applied to a gene means any gene derived from the same or a different microbe having the same function and may have significant sequence similarity.

“Transcriptional and translational control sequences” are DNA regulatory sequences, such as promoters, enhancers, terminators, and the like, that provide for the expression of a coding sequence in a host cell. In eulcaryotic cells, polyadenylation signals are control sequences.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “genomic DNA” refers to total DNA from an organism.

The term “total RNA” refers to non-fractionated RNA from an organism.

The term “probe” refers to a single-stranded nucleic acid molecule that can base pair with a complementary single stranded target nucleic acid to form a double-stranded molecule.

The term “label” will refer to any conventional molecule which can be readily attached to mRNA or DNA and which can produce a detectable signal, the intensity of which indicates the relative amount of hybridization of the labeled probe to the DNA fragment. Preferred labels are fluorescent molecules or radioactive molecules. A variety of well-known labels can be used.

The term “complementary” is used to describe the relationship between nucleotide bases that are capable to hybridizing to one another. For example, with respect to DNA, adenosine is complementary to thymine and cytosine is complementary to guanine. Accordingly, the instant invention also includes isolated nucleic acid fragments that are complementary to the complete sequences as reported in the accompanying Sequence Listing as well as those substantially similar nucleic acid sequences.

The term “growth cycle” as applied to a cell refers to the metabolic cycle through which a cell moves in culture conditions. The cycle may be divided into various stages known as the exponential phase, the end of exponential, and the stationary phase.

The term “exponential growth”, “exponential phase growth”, “log phase” or “log phase growth” refer to the rate at which microorganisms are growing and dividing. When growing in log phase microorganisms are growing at the maximal rate possible given their genetic potential, the nature of the medium, and the conditions under which they are grown. Microorganism rate of growth is constant during exponential phase and the microorganism divides and doubles in number at regular intervals. Cells that are “actively growing” are those that are growing in log phase.

The term “stationary phase” refers to the growth cycle phase where cell growth in a culture slows or even ceases. In Bacillus subtilis, T0 represents the end of the exponential growth phase or the beginning of the stationary phase. T1 means one hour after T0 or one hour into the stationary phase. T3 means three hours from T0 or three hours into the stationary phase.

The term “growth-altering environment” refers to energy, chemicals, or living things that have the capacity to either inhibit cell growth or kill cells. Inhibitory agents may include but are not limited to mutagens, antibiotics, UV light, gamma-rays, x-rays, extreme temperature, phage, macrophages, organic chemicals and inorganic chemicals.

“State of the cell” refers to metabolic state of the organism when grown under different conditions.

The term “alkyl” will mean a univalent group derived from alkanes by removal of a hydrogen atom from any carbon atom: CₙH₂ₙ₊₁—. The groups derived by removal of a hydrogen atom from a terminal carbon atom of unbranched alkanes form a subclass of normal alkyl (n-alkyl) groups: H[CH₂]ₙ—. The groups RCH₂—, R₂CH— (R not equal to H), and R₃C— (R not equal to H) are primary, secondary and tertiary alkyl groups respectively.

The term “alkenyl” will mean an acyclic branched or unbranched hydrocarbon having one carbon-carbon double bond and the general formula CₙH₂ₙ. Acyclic branched or unbranched hydrocarbons having more than one double bond are alkadienes, alkatrienes, etc.

The term “alkylidene” will mean the divalent groups formed from alkanes by removal of two hydrogen atoms from the same carbon atom, the free valencies of which are part of a double bond (e.g. (CH₃)₂C=propan-2-ylidene).

The term “DNA microarray” or “DNA chip” means the assembling of PCR products of a group of genes or all genes within a genome on a solid surface in a high density format or array. General methods for array construction and use are available (see Schena M., Shalon D., Davis R. W., Brown P. O., Quantitative monitoring of gene expression patternswith a complementary DNA microarray. Science. 1995 Oct. 20; 270(5235): 467-70 and http://cmgm.stanford.edu/pbrown/mguide/index.html). A DNA microarray allows for the analysis of gene expression patterns or profiles of many genes to be performed simultaneously by hybridizing the DNA microarray comprising these genes or PCR products of these genes with cDNA probes prepared from the sample to be analyzed. DNA microarray or “chip” technology permits examination of gene expression on a genomic scale, allowing transcription levels of many genes to be measured simultaneously. Briefly, DNA microarray or chip technology comprises arraying microscopic amounts of DNA complementary to genes of interest or open reading frames on a solid surface at defined positions. This solid surface is generally a glass slide, or a membrane (such as nylon membrane). The DNA sequences may be arrayed by spotting or by photolithography (see http://www.affymetrix.com/). Two separate fluorescently-labeled probe mixes prepared fiom the two sample(s) to be compared are hybridized to the microarray and the presence and amount of the bound probes are detected by fluorescence followimg laser excitation using a scanning confocal microscope and quantitated using a laser scanner and appropriate array analysis software packages. Cy3 (green) and Cy5 (red) fluorescent labels are routinely used in the art, however, other similar fluorescent labels may also be employed. To obtain and quantitate a gene expression profile or pattern between the two compared samples, the ratio between the signals in the two channels (red:green) is calculated with the relative intensity of Cy5/Cy3 probes taken as a reliable measure of the relative abundance of specific mRNAs in each sample. Materials for the construction of DNA microarrays are commercially available (Affymetrix (Santa Clara Calif.) Sigma Chemical Company (St Louis, Mo.) Genosys (The Woodlands, Tex.) Clontech (Palo Alto Calif.) and Corning (Corning N.Y.). In addition, custom DNA microarrays can be prepared by commercial vendors such as Affymetrix, Clontech, and Corning.

The term “expression profile” refers to the expression of groups of genes under a given conditions.

The term “gene expression profile” refers to the expression of an individual gene and of suites of individual genes.

Standard recombinant DNA and molecular cloning techniques used here are well known in the art and are described by Sambrook, J., Fritsch, E. F. and Maniatis, T., Molecular Cloning: A Laboratory Manual Second Edition, Cold Spring Harbor Laboratory Press, Cold Spring Harbor, N.Y. (1989) (hereinafter “Maniatis”); and by Silhavy, T. J., Bennan, M. L. and Enquist, L. W., Experiments with Gene Fusions, Cold Spring Harbor Laboratory Cold Press Spring Harbor, N.Y. (1984); and by Ausubel, F. M. et al., Current Protocols in Molecular Biology, published by Greene Publishing Assoc. and Wiley-Interscience (1987).

The present invention identifies a number of genes contained within the Bacillus subtilis genome that are responsive to various metabolic conditions or growth cycle conditions. The discovery that these genes are regulated in response to these conditions allows for their use in gene expression and in the monitoring and regulating of bioreactor health.

### Generation of Microarrays

The invention identifies a number of genes known in the art as being responsive to various conditions not heretofore appreciated. The identification of these new inducing conditions was made by means of the application of DNA mircoarray technology to the Bacillus subitilis genome. Any Bacillus species may be used, however Bacillus subtillis strain, obtained from Bacillus Genetic Stock Center (Ohio State University, Columbus, Ohio) is preferred.

The generation of DNA microarrays is common and well kcnown in the art (see for example Brown et al., U.S. Pat. No. 6,110,426). Typically generation of a microarry begins with providing a nucleic acid sample comprising mRNA transcript(s) of the gene or genes, or nucleic acids derived from the mRNA transcript(s) to be included in the array. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the MnRNA transcript and detection of such derived products is indicative of the presence and/or abundance of the original transcript in a sample. Thus, suitable samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

Typically the genes are amplified by methods of primer directed amplification such as polymerase chain reaction (PCR) (U.S. Pat. No. 4,683,202 (1987, Mullis, et al.) and U.S. Pat. No. 4,683,195 (1986, Mullis, et al.), ligase chain reaction (LCR) (Tabor et al., Proc. Acad Sci. U.S.A., 82, 1074-1078 (1985)) or strand displacement amplification (Walker et al., Proc. Natl. Acad. Sci. U.S.A., 89, 392, (1992)) for example.

Amplified ORF's are then spotted on slides comprised of glass or some other solid substrate by methods well known in the art to form a micro-array. Methods of forming high density arrays of oligonucleotides, with a minimal number of synthetic steps are known (see for example Brown et al., U.S. Pat. No. 6,110,426). The oligonucleotide analogue array can be synthesized on a solid substrate by a variety of methods, including, but not limited to, light-directed chemical coupling, and mechanically directed coupling. See Pirrung et al., U.S. Pat. No. 5,143,854 (see also PCT Application No. WO 90/15070) and Fodor et al., PCT Publication Nos. WO 92/10092 and WO 93/09668 which disclose methods of forming vast arrays of peptides, oligonucleotides and other molecules using, for example, light-directed synthesis techniques. See also, Fodor et al., Science, 251, 767-77 (1991).

The ORF's are arrayed in high density on at least one glass microscope slide. Once all the genes of ORF's from the genome are amplified, isolated and arrayed, a set of probes, bearing a signal generating label are synthesized. Probes may be randomlv generated or may be synthesized based on the sequence of specific open reading frames. Probes are typically single stranded nucleic acid sequences which are complementary to the nucleic acid sequences to be detected. Probes are “hybridizable” to the ORF's. The probe length can vary from 5 bases to tens of thousands of bases, and will depend upon the specific test to be done. Typically a probe length of about 15 bases to about 30 bases is suitable. Only part of the probe molecule need be complementary to the nucleic acid sequence to be detected. In addition, the complementrrity between the probe and the target sequence need not be perfect. Hybridization does occur between imperfectly complementary molecules with the result that a certain fraction of the bases in the hybridized region are not paired with the proper complementary base.

Signal generating labels that may be incorporated into the probes are well known in the art. For example labels may include but are not limited to fluorescent moieties, chemiluminescent moieties, particles, enzymes, radioactive tags, or light emitting moieties or molecules, where fluorescent moieties are preferred. Most preferred are fluorescent dyes capable of attaching to nucleic acids and emitting a fluorescent signal. A variety of dyes are known in the art such as fluorescein, texas red, and rhodamine. Preferred are the mono reactive dyes cy3 (146368-16-3) and cy5 (146368-14-1) both available commercially (i.e.Amersham Pharmacia Biotech, Arlington Heights, Ill.). Suitable dyes are discussed in U.S. Pat. No. 5,814,454 hereby incorporated by reference.

Labels may be incorporated by any of a number of means well known to those of skill in the art. However, in a preferred embodiment, the label is simultaneously incorporated during the amplification step in the preparation of the probe nucleic acids. Thus, for example, polymerase chain reaction (PCR) with labeled primers or labeled nucleotides will provide a labeled amplification product. In a preferred embodiment, reverse transcription or replication, using a labeled nucleotide (e.g. dye-labeled UTP and/or CTP) incorporates a label into the transcribed nucleic acids.

Alternatively, a label may be added directly to the original nucleic acid sample (e.g., mRNA, polyA mRNA, cDNA, etc.) or to the amplification product after the synthesis is completed. Means of attaching labels to nucleic acids are well known to those of skill in the art and include, for example nick translation or end-labeling (e.g. with a labeled RNA) by kinasing of the nucleic acid and subsequent attachment (ligation) of a nucleic acid linker joining the sample nucleic acid to a label (e.g., a fluorophore).

Following incorporation of the label into the probe the probes are then hybridized to the micro-array using standard conditions where hybridization results in a double stranded nucleic acid, generatuig a detectable signal from the label at the site of capture reagent attachment to the surface. Typically the probe and array must be mixed with each other under conditions which will permit nucleic acid hybridization. This involves contacting the probe and array in the presence of an inorganic or organic salt under the proper concentration and temperature conditions. The probe and array nucleic acids must be in contact for a long enough time that any possible hybridization between the probe and sample nucleic acid may occur. The concentration of probe or array in the mixture will determine the time necessary for hybridization to occur. The higher the probe or array concentration the shorter the hybridization incubation time needed. Optionally a chaotropic agent may be added. The chaotropic agent stabilizes nucleic acids by inhibiting nuclease activity. Furthermore, the chaotropic agent allows sensitive and stringent hybridization of short oligonucleotide probes at room temperature [Van Ness and Chen (1991) Nucl. Acids Res. 19:5143-5151]. Suitable chaotropic agents include guanidinium chloride, guanidinium thiocyanate, sodium thiocyanate, lithium tetrachloroacetate, sodium perchlorate, rubidium tetrachloroacetate, potassium iodide, and cesium trifluoroacetate, among others. Typically, the chaotropic agent will be present at a final concentration of about 3 M. If desired, one can add formamide to the hybridization mixture, typically 30-50% (v/v).

Various hybridization solutions can be employed. Typically, these comprise from about 20 to 60% volume, preferably 30%, of a polar organic solvent. A common hybridization solution employs about 30-50% v/v formamide, about 0.15 to 1 M sodium chloride, about 0.05 to 0.1 M buffers, such as sodium citrate, Tris-HCl, PIPES or HEPES (pH range about 6-9), about 0.05 to 0.2% detergent, such as sodium dodecylsulfate, or between 0.5-20 mM EDTA, FICOLL (Pharmacia Inc.) (about 300-500 kilodaltons), polyvinylpyrrolidone (about 250-500 kdal), and serum albumin. Also included in the typical hybridization solution will be unlabeled carrier nucleic acids from about 0.1 to 5 mg/mL, fragmented nucleic DNA, e.g., calf thymus or salmon sperm DNA, or yeast RNA, and optionally from about 0.5 to 2% wt./vol. glycine. Other additives may also be included, such as volume exclusion agents which include a variety of polar water-soluble or swellable agents, such as polyethylene glycol, anionic polymers such as polyacrylate or polymethylacrylate, and anionic saccharidic polymers, such as dextran sulfate. Methods of optimizing hybridization conditions are well lmown to those of skill in the art (see, e.g., Laboratory Techniques in Biochemistry and Molecular Biology, Vol. 24: Hybridization With Nucleic Acid Probes, P. Tijssen, ed. Elsevier, N.Y., (1993)) and Maniatis, supra.

### Identification of Responsive CGenes

The basis of gene expression profiling via micro-array technology relies on comparing an organism under a variety of conditions that result in alteration of the genes expressed. Within the context of the present invention a single population of cells was exposed to a variety of stresses that resulted in the alteration of gene expression. The stresses or induction conditions analyzed included 1) oxygen deprivation 2) the combination of oxygen deprivation and presence of nitrite and 3) reaching the stationary growth phase. Non-stressed cells are used for generation of “control” arrays and stressed cells are used to generate an “experimental”, “stressed” or “induced” arrays.

Using the above described method of DNA microarray technology and comparing induced vs. non-induced cultures it was determined that the genes narGHJI, csn, yncM, yvyD, yva WXY, ydjL, sunA, and yolIJK are induced in the absence of oxygen in the log or exponential phase of the Bacillus cell cycle. Similarly it was determined that absence of oxygen combined with the presence of nitrite was sufficient to upregulate or induce the genes feuABC, ykuNOP, and dhbABC. Typically the concentration of nitrite is from about 1 mM to about 10 mM in the medium. In these instances the necessary elements for induction include both the lack of oxygen and growth in the log phase. Either the addition of oxygen or reaching the stationary growth phase resulted in the down regulation of these genes.

Additionally it was discovered that a number of genes were highly induced at various times in the stationary phase of the cell growth cycle. For example, reaching T0 of the stationary phase under aerobic conditions was sufficient to upregulate the genes ycgMN, dhaS rapF, rapG, raph, rapIC yqhIJ yveKLMNOPQST, yhfRSTUV, csn, yncM, yvyD, yvaVXT, ydjL, sunA, and yolIJK. Similarly reaching T1 of the stationary phase under aerobic conditions was sufficient to upregulate the genes acoABCL, andglvAC. Reaching T3 of the stationary phase under aerobic conditions was sufficient to upregulate the genes yxjCDEF, yngEFGHI, yjmCDEFG, ykfABCD, and yodOPRST

In addition to the discovery of the induction conditions for the above mentioned genes, it was further discovered that a number of genes were down regulated at very specific times during the growth cycle. For example, alsT and yxeKLMN regions are down-regulated upon entering the stationary phase.

It will be appreciated by the skilled person that the genes of the present invention have homologues in a variety of Bacillus species and the use of the genes for heterologus gene expression and the monitoring of bioreactor health and production are not limited to those genes derived from Bacillus subitilis but extend to homologues in any Bacillus species if they are present. For example the invention encompasses homologues derived from species including, but not limited to Bacillus subtillus, Bacillus thuringiensis, Bacillus anthracis, Bacillus cereus, Bacillus brevis, Bacillus megaterium, Bacillus intermedius, Bacillus thermoamyloliquefaciens, Bacillus amyloliquefaciens, Bacillus circulans, Bacillus licheniformis, Bacillus macerans, Bacillus sphaericus, Bacillus stearothermophilus, Bacillus laterosporus, Bacillus acidocaldarius, Bacillus pumilus, and Bacillus pseudofirmus. Although all of the genes of the present invention have been identified in the Bacillus subtilis genome (Kunst et al., Nature 390 (6657), 249-256 (1997) homologs of csn for example have been identified in Bacillus circulans, and Bacillus ehimensis (Shimosalca et al., Appl. Microbiol Biotechnol. (2000), 54(3), 354-360; Masson et al., Gene (1994), 140(1), 103-7 and in Bacillus aimyloliquefaciens (Seki et., Adv. Chitin Sci. 20 (1997), 2, 284-289.

The function of the instant genes and the conditions under which they are up-regulated or down-regulated are given in Table 1 below.

TABLE 1

| Gene or Gene | | | |
|---|---|---|---|
| Cluster Name* | Function | Up-regulated | Down-regulated |
| NarGHJI | Nitrate | in Log Phase | Stationary Phase |
| | reduction | under oxygen- | under oxygen- |
| | | limiting | limitation |
| | | conditions | conditions |
| csn | chitosanase | O2 depletion | |
| | | in Log Phase | |
| | | or | |
| | | +O2 in | |
| | | stationary | |
| | | Phase in the | |
| yncM | Unknown | O2 depletion | |
| | | in Log Phase | |
| | | or | |
| | | +O2 in | |
| | | stationary | |
| | | Phase | |
| yvyD | | O2 depletion | |
| | | in Log Phase | |
| | | or | |
| | | +O2 in | |
| | | stationary | |
| | | Phase | |
| yvaWXY | | O2 depletion | |
| | | in Log Phase | |
| | | or | |
| | | +O2 in | |
| | | stationary | |
| | | Phase | |
| ydjL | | O2 depletion | Stationary Phase |
| | | in Log Phase | |
| | | or | |
| | | +O2 in | |
| | | stationary | |
| | | Phase | |
| sunA | Sublancin | O2 depletion | |
| | lantibiotic | in Log Phase | |
| | | or | |
| | | +O2 in | |
| | | stationary | |
| | | Phase (T0 & | |
| | | T1) | |
| yolIJK | Modification | O2 depletion | |
| | of SunA | in Log Phase | |
| | | or | |
| | | +O2 in | |
| | | stationary | |
| | | Phase (T0 & | |
| | | T1) | |
| feuABC | Fe transport | O2 limiting- | |
| | | condition and | |
| | | in the | |
| | | presence of | |
| | | nitrite | |
| ykuNOP | Unknown | O2 limiting- | |
| | | condition and | |
| | | in the | |
| | | presence of | |
| | | nitrite | |
| dhbABC | Fe uptake | O2 limiting- | |
| | | condition and | |
| | | in the | |
| | | presence of | |
| | | nitrite | |
| dhaS | aldehyde | Aerobic, | |
| | dehydrogenase | stationary | |
| | | (T0, T1, T3) | |
| rapF | response | Aerobic, | |
| | regulator | stationary | |
| | aspartate | (T0, T1, T3) | |
| | phosphatase | | |
| rapG | response | Aerobic, | |
| | regulator | stationary | |
| | aspartate | (T0, T1, T3) | |
| | phosphatase | | |
| rapH | response | Aerobic, | |
| | regulator | stationary | |
| | aspartate | (T0, T1, T3) | |
| | phosphatase | | |
| rapK | response | Aerobic, | |
| | regulator | stationary | |
| | aspartate | (T0, T1, T3) | |
| | phosphatase | | |
| yqhIJ | Possibly | Aerobic, | |
| | involved in | stationary | |
| | amino acide | (T0, T1, T3) | |
| | biosynthesis | | |
| yveKLMNOPQST, | Polysaccharide | Aerobic, | |
| | biosythesis | stationary | |
| | | (T0, T1) | |
| yhfRSTUV | unknown | Aerobic, | |
| | | stationary | |
| | | (T0) | |
| acoABCL, | Acetoin | Aerobic, | |
| | metablism | stationary | |
| | | (T1, T3) | |
| glvAC | glvA 6- | Aerobic, | |
| | phospho- | stationary | |
| | alpha- | (T1) | |
| | glucosidase | | |
| yxjCDEF | unknown | Aerobic, | |
| | | stationary | |
| | | (T3) | |
| yngEFGHI | unknown | Aerobic, | |
| | | stationary | |
| | | (T3) | |
| yjmCDEFG | unknown | Aerobic, | |
| | | stationary | |
| | | (T3) | |
| ykfABCD | unknown | Aerobic, | |
| | | stationary | |
| | | (T3) | |
| yodOPRST | . YodO, lysine | Aerobic, | |
| | 2,3-amino- | stationary | |
| | mutase | (T3) | |
| | Rest unknown | | |
| ycgMN | Possible | Aerobic | |
| | proline | stationary | |
| | biosynthesis | (T1, T0, T3) | |
| alsT | sodium/proton- | | Aerobic |
| | dependent | | stationary |
| | alanine carrier | | |
| | (alsT) | | |
| yxeKLMN | Similar to | | Aerobic |
| | amino acid | | stationary |
| | transporter | | |
| | or mono- | | |
| | oxygenase | | |

*All genes have been identified in the complete sequence of the Bacillus subtilis genome
(Kunst et al., Nature 390 (6657), 249-256 (1997)

Although narGHJI and acoABCL have been previously characterized using DNA microarray technology, Applicants have been able to compare the relative fold induction of the genes with more than 4,000 other genes in the genome to derive new functional information. For example it was seen that the narGHJI was the highest induced region under anaerobic conditions in the log phase. The acoABCL is the highest induced region after one hour into the stationary phase. These findings demonstrate that the promoter regions from these genes may be used to regulate gene expression or they may function as diagnostic markers.

### Expression Profiles To Monitor Biomass.

The genes of the present invention may be used in a variety of formats for the monitoring of the state of biomass in a reactor.

A gene expression profile is a reflection of the environmental conditions within which a cell is growing at anyone particular time. As a result, these profiles or patterns can be used as markers to describe the metabolic state of the cells. For example, an increase in mRNA levels for ycgMN, rapF, rapK, rapH, rapG, yvyD, yvaWXY, sunA, yncM, ydjL, yhJRSTUV genes and a reduction in alsT and yxeKLAN will indicate the cell is experiencing nutrient limitation since their expression levels start to change at the end of exponential phase. If the DNA regions yjmCDEFG, ykfABCD, yngEFGHI, and yxjDDEF show increased mRNA levels, that will suggest a more severe state of nutrient limitation since they are normally expressed three hours into the stationary phase. Similarly an increase in transcription for sunA, yolIJK, yvaWXY, ydjL, yvyD, csn, and yncM, but not other stationary phase genes, will indicate a limitation in oxygen supply to the cell.

Formats for using these genes for biomass monitoring will vary depending on the type of fermentation to be monitored and will include but is not limited to DNA microarrv analysis; northern blots [Krumlauf, Robb, Methods Mol. Biol. (Totowa, N.J.) (1991), 7 (Gene Transfer Expression Protocols), 307-23,] primer extension, and nuclease protection assays [Walmsley et al., Methods Mol. Biol. (Totowa, N.J.) (1991), 7 (Gene Transfer Expression Protocols), 271-81] or other mRNA quantification procedures. Methods of gene expression monitoring with DNA microarrays typically involve (1) construction of DNA microarray for Bacillus subtilis (2) RNA isolation, labeling and slide hybridization of a nucleic acid target sample to a high density array of nucleic acid probes, and (3) detecting and quantifying the amount of target nucleic acid hybridized to each probe in the array and calculating a relative expression. Hybridization with these arrays permits simultaneous monitoring of the various members of a gene family and subsequently allows one to optimize production yield in a bioreactor by monitoring the state of the biomass.

Furthermore, the expression monitoring method of the present invention allows for the development of “dynamic” gene database that defmes a gene's function and its interaction with other genes. The identified genes can be used to study the genes responsible for the inactivation and expression analysis of the unanalyzed genes in different regions of Bacillus subtilis genome. The results of this kind of analysis provides valuable information about the necessity of the inactivated genes and their expression patterns during growth in different conditions.

Additionally, the genes which have been identified by the present invention can be employed as promoter candidates and diagnostic markers for the metabolic state of the organism and potential stress factors or limitations of nutrients during growth. For example, an optimized process for the production of a specific bio-based material can be developed with the promoters and gene expression patterns in the present invention. Such a process could involve culture media change, oxygen input, nutrient composition, environmental stress (such as pH and temperature changes), overproduction of a particular product or expression of a foreign gene product. Accordingly, through the use of such methods, the present invention may bejsed to monitor global expression profiles which reflect the state of the cell.

### Regulated Gene Expression

The genes of the present invention may be used to effect the regulated expression of chimeric genes in various Bacillus sp. under specific induction conditions or at a specific point in the cell growth cycle. Useful chimeric genes will include the promoter region of any one of the inducible genes defined herein, operably linked to a coding region of interest to be expressed in a Bacillus host. Any host that is capable of accommodating the promoter region is suitable including but not limited to Bacillus subtillus, Bacillus thuringiensis, Bacillus anthracis, Bacillus cereus, Bacillus brevis, Bacillus megaterium, Bacillus intermedius, Bacillus thermoamyloliquefaciens, Bacillus amyloliquefaciens, Bacillus circulans, Bacillus licheniformis, Bacillus macerans, Bacillus sphaericus, Bacillus stearothermophilus, Bacillus laterosporus, Bacillus acidocaldarius, Bacillus pumilus, and Bacillus pseudofirmus.

Coding regions of interest to be expressed in the recombinant Bacillus host may be either endogenous to the host or heterologous and must be compatible with the host organism. Genes encoding proteins of commercial value are particularly suitable for expression. For example, coding regions of interest may include, but are not limited to those encoding viral, bacterial, fungal, plant, insect, or vertebrate, including mammalian polypeptides and may be, for example, structural proteins, enzymes, or peptides. A particularly preferred, but non-limiting list include, genes encoding enzymes involved in the production of isoprenoid molecules, genes encoding polyhydroxyalkanoic acid (PHA) synthases (phaE; Genbank Accession No. GI 1652508, phaC; Genbank Accession No. GI 1652509) from Synechocystis or other bacteria, genes encoding carotenoid pathway genes such as phytoene synthase (crtB; Genbank Accession No. GI 1652930), phytoene desaturase (crtD; Genbank Accession No. GI 1652929), beta-carotene ketolase (crto; Genbank Accession No. GI 1001724); and the like, ethylene forming enzyme (efe) for ethylene production, pyruvate decarboxylase (pdc), alcohol dehydrogenase (adh), cyclic terpenoid syntahses (i.e. limonene synthase, pinene synthase, bomyl synthase, phellandrene synthase, cineole synthase, and sabinene synthase) for the production of terpenoids, and taxadiene synthase for the production of taxol, and the like. Genes encoding enzymes involved in the production of isoprenoid molecules include for example, geranyfgeranyl pyrophosphate synthase (crtE; Genbank Accelon No. GI 1651762), solanesyl diphosphate synthase (sds; Genbank Accession No. GI 1651651), which can be expressed in Bacillus to exploit the high flux for the isoprenoid pathway in this organism. Genes encoding polyhydroxyalkanoic acid (PHA) synthases (phaE, phaC) may be used for the production of biodegradable plastics.

The initiation regions or promoters for construction of the chimera to be expressed will be derived from the inducible genes identified herein. The promoter regions may be identified from the sequence of the inducible genes and their homologues (see Table 1) and isolated according to common methods (Maniatis supra). Once the promoter regions are identified and isolated they may be operably linked to a coding region of interest to be expressed in suitable expression vectors.

Examples of sequence-dependent protocols for homologue identification include, but are not limited to, methods of nucleic acid hybridization, and methods of DNA and RNA amplification as exemplified by various uses of nucleic acid amplification technologies [e.g., polymerase chain reaction, Mullis et al., U.S. Pat. No. 4,683,202; ligase chain reaction (LCR), Tabor, S. et al., Proc. Acad Sci. USA 82, 1074, (1985)] or strand displacement amplification [SDA, Walker, et al., Proc. Natl. Acad. Sci. U.S.A., 89, 392, (1992)].

Generally two short segments of the instant sequences may be used in polymerase chain reaction protocols to amplify longer nucleic acid fragments encoding homologous genes from DNA or RNA. The polymerase chain reaction may also be performed on a library of cloned nucleic acid fragments wherein the sequence of one primer is derived from the instant nucleic acid fragments, and the sequence of the other primer takes advantage of the presence of the polyadenylic acid tracts to the 3′ end of the mRNA precursor encoding microbial genes.

Alternatively the instant sequences may be employed as hybridization reagents for the identification of homologues. The basic components of a nucleic acid hybridization test include a probe, a sample suspected of containing the gene or gene fragment of interest, and a specific hybridization method. Probes of the present invention are typically single stranded nucleic acid sequences which are complementary to the nucleic acid sequences to be detected. Probes are “hybridizable” to the nucleic acid sequence to be detected.

Vectors or cassettes useful for the transformation of suitable Bacillus host cells are well known in the art. Typically the vector or cassette contains sequences directing transcription and translation of the relevant gene, a selectable marker, and sequences allowing autonomous replication or chromosomal integration. Suitable vectors comprise a region 5′ of the gene which harbors transcriptional initiation controls and a region 3′ of the DNA fragment which controls transcriptional termination. It is most preferred when both control regions are derived from genes homologous to the transformed host cell, although it is to be understood that such control regions need not be derived from the genes native to the specific species chosen as a production host. Termination control regions may also be derived from various genes native to the preferred hosts. Optionally, a termination site may be unnecessary, however, it is most preferred if included.

Application of integration vectors for genetic manipulation is very well established and widely used in Bacillus subtilis (M. Perego, 1993, In Bacillus subtilis and Other Gram-Positive Bacteria, p.615-624.). Alternatively, the promoters to be used can be cloned into a plasmid which is capable of transforming and replicating itself in Bacillus subtilis (L. Janniere, et al, In Bacillus subtilis and Other Gram-Positive Bacteria, p. 625-644; Nagarajan et al, 1987, U.S. Pat. No. 4,801,537). The gene to be expressed can then be cloned downstream from the promoter. Once the recombinant Bacillus sp. is established, gene expression can be accomplished by the conditions such as oxygen-limitation, nitrite addition and others.

Optionally it may be desired toproduce the instant gene product as a secretion product of the transformed hdst. Secretion of desired proteins into the growth media has the advantages of simplified and less costly purification procedures. It is well known in the art that secretion signal sequences are often usefuil in facilitating the active transport of expressible proteins across cell membranes. The creation of a transformed host capable of secretion may be accomplished by the incorporation of a DNA sequence that codes for a secretion signal which is functional in the host production host. Methods for choosing appropriate signal sequences are well known in the art (see for example EP 546049;WO 9324631). The secretion signal DNA or facilitator may be located between the expression-controlling DNA and the instant gene or gene fragment, and in the same reading frame with the latter.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions.

### General Methods

Standard recombinant DNA and molecular cloning techniques used in the Examples are well known in the art and are described by Sambrook, J., Fritsch, E. F. and Maniatis, T. Molecular Cloning.: A Laboratory Manual; Cold Spring Harbor Laboratory Press: Cold Spring Harbor, (1989) (Maniatis) and by T. J. Silhavy, M. L. Bennan, and L. W. Enquist, Experiments with Gene Fusions, Cold Spring Harbor Laboratory, Cold Spring Harbor, N.Y. (1984) and by Ausubel, F. M. et al., Current Protocols in Molecular Biology, pub. by Greene Publishing Assoc. and Wiley-lnterscience (1987).

The meaning of abbreviations is as follows: “hr” means hour(s), “min” means minute(s), “sec” means second(s), “d” means day(s), “mL” means milliliter(s), “μL” means microliter(s), “nL” means nanoliter(s), “μg” means microgram(s), “ng” means nanogram(s), “nM” means millimole(s), “μM” means micromole(s).

### Media and Culture Conditions

Materials and methods suitable for the maintenance and growth of bacterial cultures were found in Experiments in Molecular Genetics (Jeffrey H. Miller), Cold spring Harbor Laboratory Press (1972), Manual of Methods for General Bacteriology (Phillip Gerhardt, R. G. E. Murray, Ralph N. Costilow, Eugene W. Nester, Willis A. Wood, Noel R. Krieg and G. Briggs Phillips, eds), pp. 210-213, American Society for Microbiology, Washington, D.C. or Thomas D. Brock in Biotechnology: A Textbook of Industrial Microbiology, Second Edition (1989) Sinauer Associates, Inc., Sunderland Mass. All reagents and materials used for the growth and maintenance of bacterial cells were obtained from Aldrich Chemicals (Milwaukee, Wis.), DIFCO Laboraoties (Detroit, Mich.), Gibco/BRL (Gaithersburg, Md.), or Sigima Chemical Company (St. Louis, Mo.) unless otherwise specified.

### Molecular Biology Techniques

Methods for agarose gel electrophoresis were performed as described in Sambrook, J., et al., Molecular Cloning A Laboratory Manual, Second Edition, Cold Spring Harbor Laboratory Press (1989). Polymerase Chain Reactions (PCR) techniques were found in White, B., PCR Protocols: Current Methods and Applications, Volume 15(1993) Humana Press Inc.

### Example 1

### Application of DNA Microarray Technology in Bacillus subtilis

Example 1 describes a procedure for the use of DNA microarray in Bacillus subtilis following growth of the cells in different growth medium. The signal intensity of each spots in the array was used to determine genome-wide gene expression patterns of this organism.

### Bacillus subtilis Strain and Culture Media

Bacillus subtilis strain 168 (a derivative JH642) was obtained from Bacillus Genetic Stock Center (Ohio State University, Columbus, Ohio). Cells were routinely grown at 37° C. in the following media: 2×YT medium (Gibco BRL, Gaithersburg, Md.) or Schaeffer's sporulation medium. One liter of the Schaeffer's medium contains the following ingedient: 8 g Bacto-nutrient broth, 1 g ofKCl, 0.12 g ofMgSO₄.7H₂O, 0.5 ml of 1.0 MNaOH, 1 ml of 1.0 M Ca(NO₃)₄, 1 ml of 0.01 M MnCl₂, and 1 ml of 1.0 mM FeSO₄.

### Construction of DNA Microarray for Bacillus subtilis

The oligonucleotides for all 4,100 ORFs of the Bacillus subtilis genome were purchased from Genosys (Woodlands, Tex.). The HotStart PCR kit from Qiagen (Valencia, Calif.) was used for all PCR reactions. The cycling conditions were as follows: 30 seconds of annealing at 55° C., 2 minutes of elongation at 72° C., and 30 seconds of denaturing at 95° C. The PCR products were purified with the QIAquick Multiwell PCR purification kit from Qiagen and the quality of the PCR reactions was checked by electrophoresis on an agarose gel (1%). Each image was stored in a database and the observed sizes of PCR products were automatically comparedto the expected value. This information was also used as a reference to check the quality of hybridization at a later stage. After two rounds of PCR reactions, about 95% of the PCR reactions were successful and the remaining ORFs were amplified with another set of oligonucleotides. If an ORF was larger than 3 kb, only a portion of the gene (2 kb or less) was amplified. A total of 4,020 PCR products were obtained. These PCR products were spotted onto sodium thiocyanate optimized Type 6 slides (Amersham Pharmacia Biotech, Piscataway, N.J.) with the Molecular Dynamics Generation III spotter (Sunnyvale, Calif.). Each of the 4,020 PCR products was spotted in duplicate on a single slide.

Each array slide also contained 10 different internal controls consisting different 1.0 kb lambda DNA fiagments. The PCR product of each control was spotted in three different locations in the array. Every control fragment also contained a T7 promoter generated by PCR reaction. PCR products were directly used to generate RNAs with the in vitro transcription kit (Ambion, Austin, Tex.). An equal amount of control mRNA mixture was spiked into the two total RNA samples before each labeling.

### RNA Isolation, Labeling and Slide Hybridization

Total RNA was isolated from Bacillus subtilis with the Qiagen RNeasy Mini kit. The cell culture was harvested by centrifugation with a Bechman table top centifuge (Beckman Instruments, Fullerton, Calif.). The speed of centrifuge was brought up to 9,000 rpm and then stop immediately. Cells were suspended directly in RLT buffer and placed in a 2 ml tube with ceramic beads from the FastRNA kit (Bio101, Vista, Calif.). The tube was shaken for 40 seconds at the speed setting of 4.0 in a bead beater (FP120 FastPrep cell disrupter, Savant Instruments, Inc., Holbrook, N.Y.). Residue DNA was removed on-column with Qiaen RNase-free DNase. To generate cDNA probes with reverse transcriptase, 10 to 15 ug of RNA was used for each labeling reaction. The protocol for labeling was similar to the one previously described for yeast (DeRisi, J. L., V. P Iyer, and P. O. Brown, 1997, Exploring the metabolic and genetic control of gene expression on a genomic scale, Science. 278:680-686). For this work, random hexamers (Gibco BRL) were used for priming and the fluorophor Cy3-dCTP or Cy5-dCTP (Amersham Pharmacia Biotech) was used for labeling. After labeling, RNA was removed by NaOH treatment and cDNA was immediately purified with a Qiagen PCR Mini kit. The efficiency of labeling was routinely monitored by the absorbance at 260 nm (for DNA concentration), 550 nm (for Cy5), and 650 nm (for Cy3).

Each total RNA preparation was labeled with both Cy3-dCTP and Cy5-dCTP. To hybridize a single glass slide, the Cy3-labeled probe from one growth condition was mixed with the Cy5-labeled probe from another and vice versa. As a result, each experiment required two slides. An equal amount of Cy3- and Cy5-labeled probes based on the incorporated dye concentration was applied to each slide. The amount of cy3- and Cy5-labeled probe was determined by the extinction coefficient at 550 nm (for Cy5 dye) and 650 nm (Cy3). The hybridization was carried out at 37° C. overnight with Microarray Hybridization Buffer containing formamide (Amersham Pharmacia Biotech). Slides were washed at 15 min intervals, once with a solution containing 2×SSC and 0.1% SDS at 37° C. and three times with a solution containing 0.1×SSC and 0.1% SDS at room temperature. The slides were then rinsed with 0.1×SSC and dH₂O. After drying under a stream of N₂, the slides were scanned for fluorescent intensity of both Cy5 and Cy3 fluors. The signal from each spot in the array was quantified using ArrayVision software from Molecular Dynamics.

Data Analysis and Presentation. There are two ways to calculate the signal intensity of each spot. One is the normalized density×area (nD×A), which is the fluorescence units (after background subtraction) divided by the reference. The reference is the mean D×A-background of all elements in the array. The second signal intensity is the D×A after background subtraction. Normalization was carried out with internal controls. The purpose of normalization in the first case is to correct the errors generated due to slide and slide variation and difference in the efficiency of Cy5 and Cy3 incorporation so that data generated within the slide and from different slides can be compared directly. This method is based on total mRNA signals in the array and assumed that less than 10% of the population changed between the two conditions. The purpose of using D×A and normalization with internal controls is to measure the changes in mRNA levels when more than 10% of the total population has been changed. This method is based on total RNA level.

The ratio of intensity for Cy-3/Cy-5 or Cy-5/Cy-3 from two slides of each dye swap hybridization was averaged as one independent experiment. Data were obtained from at least three independent experiments. The ratio of spot intensity represents the relative abundance of mRNA levels under the conditions studied. The levels of mRNA often reflects fold of induction or reduction of a particular DNA region.

### Example 2

### Identification of Anaerobically Induced DNA Region in Bacillus subtilis at Exponential Phase

Using a Bacillus subtilis DNA microarray prepared according to the methods described in Example 1, applicants have identified promoters that can be employed for different level of gene expression in Bacillus subtilis and like organisms with oxygen-limiting environment as the induction conditions. This Example describes the identification of anaerobically induced genes and their corresponding promoters in Bacillus subtilis when grown in 2×YT medium. Cells grown at exponential were used.

Specifically, Bacillus subtilis strains were grown at 37° C. in 2×YT medium supplemented with 1% glucose and 20 mM K₃PO₄ (pH 7.0). For aerobic growth, 20 ml prewarmed medium was inoculated with 0.1 ml of overnight culture (1:200 dilution) in a 250 ml flask placed on a rotary platform at the speed of 250 rpm. For anaerobic growth, 120 ml prewarmed medium was placed in a 150 ml serum bottle. Three anaerobic growth conditions were tested: anaerobic growth with nitrate as the alternative electron acceptor, anaerobic growth with nitrite as the alternative electron acceptor, and fermentative growth without the presence of nitrate or nitrite. Potassium nitrate at a concentration of 5 mM or potassium nitrite at a concentration 2.5 mM was added if used. To create an anaerobic environment, the serum bottle was capped with a Teflon coated stopper and the gas phase was flushed and filled with argon gas.

To isolate RNA from the exponential cultures, samples were talcen at 0.4 O.D. at 600 nm for aerobic cultures, 0.25 O.D. for cultures grown on nitrate, 0.15 O.D. for cultures grown on nitrite, 0.12 O.D. for cultures grown with no amendments and 0.3 O.D. if pyruvate was added during fermentative growth. Total RNA was isolated and labeled with fluorescent dyes as described in Example 1. Each hybridization consisted of aerobic and one of the various anaerobic probes, containing either nitrate, nitrite or no amendment. If the ratio between anaerobic and aerobic samples was high, it indicated that a particular gene or DNA region was idced ude anacrobic conditions. With this DNA microarray technology, the highest induced region in all anaerobic conditions was narGHJI after all the expression patterns of 4,020 genes were examined. The narGHJI region has been shown to be induced under anaerobic conditions, but only with the DNA microarray techniques that the level of induction relative to all other genes can be determined. The neW anaerobic genes identified by this technique were ydjL, csn, yvyD, yvaW, yvaX, and yvaY. These genes have not been characterized and many of them are unknown. Surprisingly, there were three DNA regions that were specifically induced changes in growth conditions when nitrite was used as the electron acceptor. They include dhb, ykuNOP, and feu regions. This unique characteristic of gene induction by nitrite can be used as a mean to design expression vectors.

TABLE 2
Fold induction for genes or gene clusters involved in
nitrate and nitrite respiration in Bacillus subtilis
JH642 when grown under anaerobic conditions.

| Gene | Description | Nitrate* | Nitrite* | No Amendment* |
|---|---|---|---|---|
| narGHJI | nitrate reductase | 112-600 | 102-743 | 61-430 |
| ydjL | similar to 2,3- | 7.7 | 11.6 | 23.2 |
| | butanediol or | | | |
| | sorbitol | | | |
| | dehydrogenase | | | |
| csn | chitosanase | 13.3 | 11.4 | 27.4 |
| yncM | unknown | 4.0 | 6.4 | 21.5 |
| yvyD | unknown | 3.8 | 5.6 | 6.4 |
| yvaWXY | unknown | 5-9 | 7.0-8 | 4.5-5 |
| feuABC | Fe transport | | 10-15 | |
| dhbABC | Fe uptake | | 39-50 | |
| ykuNOP | Unknown | | 18-19 | |

*Units in fold induction vs control

### Example 3

### Identification of DNA Regions Induced at Stationary Phase with Cells Grown in the Presence of Oxygen

Using a Bacillus subtilis DNA microarray prepared according to the methods described in Example 1, applicants have identified herein promoters that can be employed for gene expression in Bacillus subtilis and like organisms when the cells reached stationary phase in the presence of oxygen. This example describes the identification of genes and their corresponding promoters induced at different stages of stationary phase when the culture was grown in the presence of oxygen in Schaeffer's, medium supplemented with 0.1% glucose and 20 mM K₃PO₄(pH 7.0).

Specifically, Bacillus subtilis strains were grown at 37° C. An aliquot of 20 ml prewarmed medium was inoculated with overnight culture to give an O.D. of 0.03 to 0.04 in a 250 ml flask placed on a rotary platform at the speed of 250 rpm. The exponential culture for RNA preparation was harvested at mid log. Cells collected at the end of exponential growth, one hour and three hours into the stationary phase were considered as T0, T1 and T3 samples, respectively. RNA isolation, labeling, and slide hybridization were carried out as described in Example 1. For hybridization, each slide contained two probes, mid-log sample and one of the stationary samples (T0, T1, or T3).

To identify genes induced at stationary phase in the presence of oxygen, the mRNA signals between exponential (log) and one of the stationary samples (T0, T1, or T3) were compared. If the ratio between stationary and log samples was high, it indicated that a particular gene or DNA region was up-regulated at stationary phase. With this DNA microarray technology, many genes were found to have an increased level of mRNA indifferent stages as shown in Table 3. Genes such as ycgAN, csn, yvaW, yvax, yvay, yncm, yvyD, and yqhIJ were all induced in all three stages. Gene such as yolI, yolJ, yolK and ydjL were mostly induced at stage T0 and T2. Expression patterns of these genes at stationary phase had not been studied before. The aco regions involved in metabolism of acetoin at stationary phase have been previously studied, but only with the DNA microarray technology that they were found to be the highest induced region at T1 stage under this growth conditions. There were quite a few clusters of genes, which were uncharacterized, that showed higher levels of mRNA three hours into the stationary phase. They included ykfABCD, yjmCDEFG, and yodLPORST. In contrast, DNA regions such as alsT and yxeKLkAN showed a reduction in mRNA levels upon entering stationary phase. This data is summaried in Table 3. Table 3 describes a selection of genes or gene clusters that showed an induction or reduction (in paranthesis) in mRNA transcriptional levels at stationary phase of Bacillus subtilis when grown in Schaeffer's medium supplemented with 0.1% glucose in the presence of oxygen.

TABLE 3

| Gene | Description | T0/log* | T1/log* | T3/log* |
|---|---|---|---|---|
| csn | | 4.2 | 21.3 | 10.6 |
| vaW | unknown | 4-11 | 19-38 | 3-21 |
| yncM | unknown | 6.61 | 28.65 | 8.71 |
| yvyD | | 8.09 | 6.73 | 10.57 |
| sunA | | 18.08 | 35.65 | |
| yolIJK | | 7-13 | 12-27 | |
| ydjL | | 14.9 | 11.4 | |
| yqhIJ | | 15-36 | 16-38 | 2-4 |
| ycgMN | | 150-300 | 15-18 | 4-6 |
| yhfRSTUV | | 8-12 | | |
| acoABCL | | | 155-358 | 19-46 |
| glvAC | | | 43-134 | |
| ykfABCD | | | | 14-26 |
| yngEFGHI | | | | 13-24 |
| yjmCDEFG | | | | 14-23 |
| yodLPORST | | | | 15-26 |
| alsT | | (15) | (29) | (41) |
| yxeKLMN | | (9-12) | (40-64) | (40-80) |

*Units in fold induction vs control

What is claimed is:

1. A method for the expression of a coding region of interest in a Bacillus sp comprising:
a) providing a transformed Bacillus sp cell having a chimeric gene comprising a nucleic acid fragment comnrising the promoter region of a Bacillus gene operably linked to a coding region of interest expressible in a Bacillus sp, wherein the nucleic acid fragment comprising the promoter region of a Bacillus gene is selected from the group consisting of narGHJT, csn, yncM, yvyD, yvaWXY, ydjL, sunA, and yolIJK and homologues thereof; and
b) growing the transformed Bacillus sp cell of step (a) in the absence of oxygen wherein the chimeric gene of step (a) is expressed.

2. A method for the expression of a coding region of interest in a Bacillus sp comprising:
a) providing a transformed Bacillus sp cell having a chimeric gene comprising a nucleic acid fragment comprising the promoter region of a Bacillus gene operably linked to a coding region of interest expressible in a Bacillus sp, wherein the nucleic acid fragment comprising the promoter region of a Bacillus gene is selected from the group consisting of narGHJI, csn, yncM, yvyD, yvaWXY, ydjL, sun,, and yolIJK and homologues thereof;
b) growing the transformed Bacillus sp. cell of step (a) in the presence of oxygen whereby the cell density is increased; and
c) removing oxygen form the transformed Bacillus sp. cell or step (b) whereby the chimeric gene is expressed.

3. A method according to claim 2 wherein after step (c) oxygen is re-supplied to the transformed Bacillus sp. cell.

4. A method according to either of claims 1 or 2 wherein the nucleic acid fragment comprising the promoter region of a Bacillus gene is selected from the group consisting of SEQ ID NOs: 1- 15.

5. A method for the expression of a coding region of interest in a Bacillus sp comprising:
a) providing a transformed Bacillus sp cell having a chimeric gene comprising a nucleic acid fragment comprising the promoter region of a Bacillus gene operably linled to a coding region of interest expressible in a Bacillus sp, wherein the nucleic acid fragment comprising the promoter region of a Bacillus gene is selected from the group consisting of feuABC, ykuVOP, and dhbABC, and homologues thereof; and
b) growing the transformed Bacillus sp cell of step (a) in the absence of oxygen and in the nresence of nitrite wherein the chimeric gene of step (a) is expressed.

6. A method according to claim 5 wherein the nucleic acid fragment comprising the promoter region of a Bacillus gene is selected from the group consisting of SEQ ID NOs: 16-24.

7. A method according to claim 6 wherein the concentration of nitrite is from about 1 mM to about 10 mM.

8. A method for the expression of a coding region of interest in a Bacillus sp comprising:
a) providing a transformed Bacillus sp cell having a chimeric gene comprising a nucleic acid fragment comprising the promoter region of a Bacillus gene operably linked to a coding region of interest expressible in a Bacillus sp, wherein the nucleic acid fragment comprising the promoter region of a Bacillus gene is selected from the group consisting of ycgMN, dhaS rapF rapG, rapH, rapK, yqhIJ, yveKLMNOPQST, yhJRSTUV csn, yncM yvyD, yvaWXY, ydjL, sunA, and yolIJK, and homologues thereof; and
b) growing the transformied Bacillus sp cell of step (a) in the presence of oxygen until the cell reaches about T0 of the stationary phase wherein the chimeric gene of step (a) is expressed.

9. A method according to claim 8 wherein the nucleic acid fragment comprising the promoter region of a Bacillus gene is selected from the group consisting of SEQ ID NOs:75, 76, 25-49, and 5-15.

10. A method for the expression of a coding region of interest in a Bacilluis sp comprising:
a) providing a transformed Bacillus sp cell having a chimeric gene comprising a nucleic acid fragment comprising the promoter region of a Bacillus gene operably linked to a coding region of interest expressible in a Bacillus sp, wherein the nucleic acid fragment comprising the promoter region of a Bacillus gene is selected from the group consisting of acoABCL, and glvAC, and homologues thereof; and
b) growing the transformed Bacillus sp cell of step (a) in the presence of oxygen until the cell reaches about T1 of the stationary phase wherein the chimeric gene of step (a) is

11. A method according to claim 10 wherein the nucleic acid fragment comprising the promoter region of a Bacillus gene is selected from the group consisting of SEQ ID NOs:4144 and 50-51.

12. A method for the expression of a coding region of interest in a Bacillus sp comprising:
a) providing a transformed Bacillus sp cell having a chimeric gene comprising a nucleic acid fragment comprising the promoter region of a Bacillus gene operably linked to a coding region of interest expressible in a Bacillus sp, wherein the nucleic acid fragment comprising the promoter region of a Bacillus gene is selected from the group consisting of yxjCDEF, yngEFGHI, yjmCDEFG, ykfABCD, and yodOPRST; and homologues thereof; and
b) growing the transformed Bacillus sp cell of step (a) in the presence of oxygen until the cell reaches about T3 of the stationary phase wherein the chimeric gene of step (a) is expressed.

13. A method according to claim 12 the nucleic acid fragment comprising the promoter region of a Bacillus gene is selected from the group consisting of SEQ ID NOs:52-74.

14. A method according to any of claims 1, 2 or 3 wherein the expression of the chimeric gene is down-regulated at T0 of the stationary phase.

15. A method according to any one of claims 1, 2, 3, 4, 8, 10 and 12 wherein the Bacillus sp. cell is selected-from the species consisting of Bacillus subtillus, Bacillus thuringiensis, Bacillus anthracis, Bacillus cereus, Bacillus brevis, Bacillus megaterium, Bacillus interinedius, Bacillus theirmoamyloliquefacieiis, Bacillus anmyloliquefaciens, Bacillus circulans, Bacillus licheniformis, Bacillus macerans, Bacillus sphaericus, Bacillus stearothermophilus, Bacillus laterosporus, Bacillus acidocaldarius, Bacillus pumilus, and Bacillus pseudofirmus.

16. The method according to any one of claims 1, 2, 3, 4, 8, 10 and 12, wherein the coding region of interest is selected from the group consisting of crtE crtB, pds, crtD, crtL, crtZ, crtX crtO, phaC, phaE, efe, pdc, adh, genes encoding limonene synthase, pinene synthase, bornyl synthase, phellandrene synthase, cineole synthase, sabinene synthase, and taxadiene synthase.

17. A method for monitoring the state of the cell metabolism of a Bacillus sp. culture comprising:
a) providing a culture of actively growing Bacillus sp. cells; and
b) measuring the expression levels of a pool of genes isolated from the Bacillus cells of step (a), the pool of genes comprising narGHJI, feuABC, ykuNOP, dhbABC, ydjL, sunA, yolIJK, csn ,yncM, yvyD, yvaWXY, yhJRSTUV, yveKLMNOPQST, dhaS, rapF, rapG, raph, rapK, ycgMN, yqhIJ, givAC, acoABCL, yxjCDEF, yngEFGHI yjmCDEFG, ykfABCD, yodOPRST, alsT, and yxeKLMN, and homologues thereof.

18. A method according to claim 17 wherein a pool of genes isolated from the Bacillus cells is selected from the group consisting of SEQ ID NOs: 1-81.

19. A method according to claim 17 wherein the measuring of gene expression levels is accomplished using a format selected from the group consisting of northern blots, nuclease protection assay or primer extension assays.

20. A method according to claim 19 wherein the measuring of gene expression levels is accomplished using a nucleic acid microarray having the genes narGHJI, feuABC, ykuNOP, dhbABC, ydjL, sunL, yolIJK, csn ,yncM, yvyD, yvaWXY, yhfRSTUV, yveKLMNOPQST, dhaS, rapF, rapG, rapH, rapK, yqhlJ, glvAC, acoABCL, yxjCDEF, yngEFGHI yjmCDEFG, ykABCD, yodOPRST, alsT, and yxeKLMN, and homologues thereof, contained therein.

21. A method according to claim 17 wherein the Bacillus sp. cell is selected from the species consisting of Bacillus subtillus, Bacillus thuringiensis, Bacillus anthracis, Bacillus cereus, Bacillus brevis, Bacillus megaterium, Bacillus intermedius, Bacillus thermoamyloliquefaciens, Bacillus amyloliquefaciens, Bacillus circulans, Bacillus licheniformis, Bacillus niacerans, Bacillus sphaericus, Bacillus stearothermophilus, Bacillus laterosporus, Bacillus acidocaldarius, Bacillus pumilus, and Bacillus pseudofirmus.

22. A method according to claim 17 wherein the actively growing culture is grown in the absence of oxygen and the expression of genes nivGHJI, ydjL, sunL, yolIJK, csn ,yncM, yvyD, and yvaWXY are up-regulated in the log phase.

23. A method according to claim 17 wherein the actively growing culture is grown in the absence of oxygen and in the presence of nitrite and the expression of genes feuABC, ykuNOP, and dhbABC are up-regulated in the log phase.

24. A method according to either of claims 22 or 23 wherein the expression of genes narGHJI is down-regulated at about T0 of the stationary phase.

25. A method according to claim 17 wherein the actively growing culture is grown in the presence of oxygen and the expression of genes ycgMN, yqhIJ, ydjL, sunA, yolIJK, csn, yncM, yvyD, yvaWXY, yhfRSTUV, yveKLMNOPQST, dhaS, rapF, rapG, rapH, rapK, are up-regulated at about T0 of the stationary phase.

26. A method according to claim 17 wherein the actively growing culture is grown in the presence of oxygen and the expression of genes, acoABCL and glvAC are up-regulated at about T1 of the stationary phase.

27. A method according to claim 17 wherein the actively growing culture is grown in the presence of oxygen and the expression of genes, yxjCDEF, yngEFGHI yjmCDEFG, ykfABCD, and yodOPRST are up-regulated at about T3 of the stationary phase.

28. A method according to claim 17 wherein the actively growing culture is grown in the presence of oxygen and the expression of genes, alsT and yxeKLAM are down-regulated at stationary phase or under nutrient-limiting conditions.


SEQUENCE LISTING (81 sequences)

SEQ ID NO: 1 - DNA, 3687 bp, Bacillus subtilis
atgaagaaaaagaaaaggagtccgttgtttaggagattgaattatttctctcctatcgaacaccattcaaataaacatagccaaactacccgcgaggatcgcgattgggagaatgtatacagaaacagatggcagtacacgaaagtcgttcgctccacccacggcgtcaactgtacagggtcttgcagctggaatatttatgtgaaaaacggaatagtcacgtgggaagggcaaaatttgaattatccatcaacaggcccggatatgcctgattttgaaccgagaggctgcccgcggggggccagtttttcatggtatatctacagcccgctccgtgtgaaatatccatacgtgcgcggtgtgctgatcaatttgtggcgggaggcattgcagacgcatcaaaatccattggaagcctggaaatcgatcgtcgaaaaccctgaaaaagcgaagtcctataaacaggcgagagggaaaggcggttttgtgcgcgctgaatggccggaggtgctgaagctgatttcagcctctctgctgtatacagtgatgaaatacgggcctgaccgaaacgtcggtttttctccgattccggccatgtccatgatcagccacgcatcaggctcccggtttatgtcgttaatcggaggccctatgctcagtttttatgactggtatgcggatcttcctccagcatccccgcaaatttggggtgaccagacggacgttccggaaagcagtgattggtacaattccggctatattatcacatggggctccaacgttccgttaacgagaacgcctgacgcgcattttttggcggaggcccgctataaaggcgctaaggtcatttcgatcagtccagattttgcggaatcctcaaagttcgcggatgactggctgagtattcgccaagggactgacggggcgcttgcgatggcgatgggtcacgttattctgcaggaattttacgtgaaccaagaaactgaacgttttattgagtacgcgaagcaatacactgattttccatttctcgtcactctgtcaaaagaaaatggcgtatacacagcgggacggtttctgcatgcgaaggacatcgggcggaagacaaagcatgatcagtggaagcctgcggtttgggatgaacagacaagttcatttgccataccccaagggacaatgggctcgcgctgggacgggcagcagaaatggaacctgcacatgattgatgaagaaaccggggaaccgattgaaccccgtctctctgtgctgggaatagaggacgaaatcggcacggtgcgcatcccgtatttttcaaatgacggaaacaaagtgctcgagcgggatcttcctattaaaaaaatgaacctgaacggtgaagaaacgtacatcacgaccgtgtttgacttgatactggctaactacggcgtgaaccggggcatcggcgaacgatcggctgtctcctatgatgaccctgagccgtttacgcctgcctggcaggaacaaatgacaggaatcaaaaaagaagctgtcgttaagattgccagagagtttgcccaaaatgcgatcgatacagacggccggtccatgattatcgtaggggccggcattaaccactggttcaactccgacacgatctaccgagcagtgttaaatcttgttttacttgtaggcgcccaaggcgtaaacggcggaggctgggcccattacgtggggcaggaaaagctccgacctgctgaagggtggcagacgattgcaactgcaaaggactgggaaggcgtgcccaagctgcaaaatggcacctcatttttctactttgcgacagatcagtggcgttatgaggaccagccgatcagtgatttggcatcaccgattgctgcttcatcccgctacaagcaccacgctgattacaatgtgctggcggcgcggctagggtggcttccgtcttacccgactttcaatcaaaatggcatcgatctgtataaagaagctgaaaaagcaggggcagcaacacctgaagacgtaggtgcgtacgtggcctcacagctccaagagaaaaaactgaaattcgcgattgaagatcctgacaatgaagtgaatttcccaaggaatctctttgtatggcgggcaaatctgatctcaagctcaggaaaagggcatgaatattttctcaagcatttgctggggacaacgaacggtttaatgaatgacgacagcgacagcatccgcccagaagaaatcaaatggcgggagcaggcgccggaaggaaagctcgacttattaatcaatcttgattttcgaatggcgggtacggcgctgtattccgatatcgtgctgccggcggcgacatggtatgaaaaacacgatctcagcagcacagatatgcatccgttcattcatccatttgctcctgcgatctcggctccgtgggaatcgaagtcagactgggatattttcaaggcgctgtcaaaagccgtttccgatctggcagaagaagtcgatatggagccggtgaaagaagtggttgcgacaccgctgctccacgacaccatgcaggaattggcccagccattcggcaaaatcaatgactggagcaaaggcgaatgtgaagccattccgggaaaaacgatgccgaacatccaagtcgttgaacgggattacaaacacatttttcataaaatgactgcacttggtccgaacgttgctttaaagccgagcggaacaaaagggatgagctggtcaatagccgatgaatatgaatcactcaaacagagactgggagaaatcacctcggacagcgtggcaaagggatgtccaaatataagtgaagcaaagcaggctgcagaagcgattttaaccctatcatccacttcgaatggaaaggtcgcagtaaaagcatgggaatcacttgaaaacatcacgaacctgaagctgaaagacctggcggaagaacgcgaggaagaatgctttacgttcgaacaaattacagcccagccgaaaacggtgatcacgtctccagcgtttaccggctctgaaaaaggagggcgcaggtattcgccgtttacaacgaatgttgaaaaattaattccgtggcggacgctgacaggcagacaatcctattatgtcgatcatgaactgatgatggaattcggtgaaacgatggcgacattcaaaccgatcctccagcatcgcccgtttctgagcaaacggcctgatcaagagggaaaagaaatcgtcctcaattatttgacgccgcataataaatggtctgtccacagcatgtattttgattctctgccgatgctgacgctgttccgcggcgggccgaccgtgtggatgaataaagatgatgcagaggacacggatatcaaagacaacgattggattgaatgcttcaaccgaaacggcgttgtcgtcgcgagagccgttttgtctcatcggattcctaaaggaatggcgtttatgcaccatgcccaggaccgccacatcaacgtgcccggcacaaagctgacgaataaccgcggaggcacccataacagcccgacaaggattcacgtcaagccgacacagatgatcggtggctacgcccagctcagctacggctttaattattacggtccaacggggaatcagcgcgacctgaacgtcgtcatccgcaagctgaaggaggtcgattggcttgaagattaa

SEQ ID NO: 2 - DNA, 1464 bp, Bacillus subtilis
ttgaagattaaagcgcaaatcggtatggtcatgaacttggataaatgcatcggctgccacacgtgcagcgtcacctgcaaaaacacgtggacaaaccgttccggtgcggaatatatgtacttcaataatgtagaaacaaagccgggcatcggctacccgaagcaatgggaggaccaggacaaatataaaggcggctggacattgaaaaaaggaaagctcgagctgaaatcgggctcgaaaaccaatcggcttgcaggccttttctataatccgaatcagccgtcaattgatgattactatgaaccttggaactatgattatgaaacattaacgaacagcccgcagaaaaaacaccagccggtagcacgcccgaaatcgtccttgacgggggatttcatgaatatcgaatggggaccgaactgggaggacgatctcgcaggcggccacattacgggacttgaagatcccaacgtacaaaagatggaggaatcgatcaaaacagaattcgatgacgtctttatgatgtatttgccccgtatttgcgagcactgcatcaacccggcatgcgtctcatcctgtccatccggcgccatgtacaaacgcgaggaggacggcattgtgcttgtggatcaaaacgcatgccgttcatggagatattgcgtctcatcctgtccttataaaaaagtctattttaactggcaaacgaacaaagcggaaaaatgcacactctgctttccgcgtttggaggcgggactgccaaccatctgctctgagacgtgtgttggcagaatccgctacctcggcgtcatgctatatgacgcggacaaagtggaggaagcggcatctgttgaaaatgaaaaggatctctaccattcccaattggacgtttttcttgatccgaatgatcctgaggttgccaaactggcaaaagaacaaggcattccggctgaatggatagaggccgcgcagcaatcaccgatctataaaatgatcattgactggaagatcgcgctgccgcttcatcctgagtaccgcacgctgccaatggtgtggtacattccgccgctcagcccgattatgaatctctttgaaggaaaaggcagccggcaaacggcggaagatatttttccggctatcgaccaaatgagaatcccgatagattatttggcgcagctgttaacagccggtgatacggatcatattcggtcaacattaaagaaaatgtctgtcatgcgccagtatatgagagcggtccagacgaataaatcaatcgatccggaactgatctccagtgtcggcttaacagaacagcaaattgaagatatgtatcggctgcttgcgattgccaaatatgatgaccgctttgtgattccgagcagccatcgagaagaagtatcagatttatacgctgaacaaggaagctgcggcttatcattttcaggcggccccggctcctgtttctaa

SEQ ID NO: 3 - DNA, 555 bp, Bacillus subtilis
atgaacaccacagaccggcaaatcacgttctctgctctttcctgtcttctctcttatccggatgaagagtggagagccgagcttcccgattggaaggctcttatccaagaaatcggcaaccggcaaatccgggagaagctgctgcactttttcgagacgtcagccagctattctccggaagcgctgattgaacactatgtctatacattcgacttcgggaaaaaaacaaatatgtatgtcacctactttaactcaggcgagcaaagggaacgcggcattgaattgctgcatttaaaaaacacatacgagcaatccggtttcctgccgacagagaaagagctgcctgattatctgccgctgatgctggaatttgctgcggctgcagaaattgaagcagcgagaagcgtgtttgagaaatatctgtccaatgtgagggagctggcatcccgtctcgaaaaaaatgacagtatatacgctgaactgctgcacgtgctgctggccgcgcttgaaaacattggcgtacgtgaaagcgttgaaggggctgttcaggcatga

SEQ ID NO: 4 - DNA, 672 bp, Bacillus subtilis
atgagcgggcagatcctctggggtattatgccatacattgtattgacaatctttatcggcggccatatttaccgctatcagcatgaccaatttggctggacggcgaaatcaagcgagctgttagaaaagaaaaaacttgcggctggcagcacactttttcactggggactgctgtgcgttgtcggcgggcatgtcatggggattctgatcccagaaggcgtgtatgcttcccttggcatttcagagcatatgtatcacaaaatggcgattggcgctggcttgccggcgggcattgcggcatgtaccggacttgtcatcctgacgtacagaaggctgtttgacaaaagaatccgcaaaacgagctcgccatccgatatccttacgctcctcctgctgctgttcatgatgctgtcaggcgttgcggccacgtttctcaacattgattcgaaaggatttgattaccggaccacagtcgggccctggttcagggaaatcgttttgttcaggcctgacgcctctttgatggagagtgtcccgctatggtttaagtttcatattgtgataggatacgtcgtttttatcctgtggccgtttacgagattggttcatgtgttcagtctgccgctcaagtatctgacccgcagctacgttgtatatcggaaacgctcgtga

SEQ ID NO: 5 - DNA, 834 bp, Bacillus subtilis
atgaaaatcagtatgcaaaaagcagatttttggaaaaaagcagcgatctcattacttgttttcaccatgttttttaccctgatgatgagcgaaacggtttttgcggcgggactgaataaagatcaaaagcgccgggcggaacagctgacaagtatctttgaaaacggcacaacggagatccaatatggatatgtagagcgattggatgacgggcgaggctatacatgcggacgggcaggctttacaacggctaccggggatgcattggaagtagtggaagtatacacaaaggcagttccgaataacaaactgaaaaagtatctgcctgaattgcgccgtctggccaaggaagaaagcgatgatacaagcaatctcaagggattcgcttctgcctggaagtcgcttgcaaatgataaggaatttcgcgccgctcaagacaaagtaaatgaccatttgtattatcagcctgccatgaaacgatcggataatgccggactaaaaacagcattggcaagagctgtgatgtacgatacggttattcagcatggcgatggtgatgaccctgactctttttatgccttgattaaacgtacgaacaaaaaagcgggcggatcacctaaagacggaatagacgagaagaagtggttgaataaattcttggacgtacgctatgacgatctgatgaatccggccaatcatgacacccgtgacgaatggagagaatcagttgcccgtgtggacgtgcttcgctctatcgccaaggagaacaactataatctaaacggaccgattcatgttcgttcaaacgagtacggtaattttgtaatcaaataa

SEQ ID NO: 6 - DNA, 753 bp, Bacillus subtilis
atggcgaaaccactatcaaaagggggaattttggtgaaaaaagtattgattgcaggtgcagtaggaacagcagttcttttcggaaccctttcatcaggtataccaggtttacccgcggcagacgctcaagtcgcaaaagcagcatccgagctgcctaacggaatcggcggccgtgtctacctgaacagtacgggcgccgtttttacagctaaaatcgtgcttcctgaaactgtcaaaaacaacgactcggtctctactccctatatttattctggctttagggcaacaagcggaactgaagccgatatcgggcttcagtacagcaaacaatacaacgtctggaagcccctcatgaaggttgggtccaaaaatgaagaaacgtacatcgaaggaaaagacaaattcacatacaataaaggcttccgccctggaagcacagtccaaatgacaatctataaaaatttaagcggcaatacgcgcatgaccctttggggaacgaacaatgacggctacaccggacggattatcacagaaattcaaggaaccaacatcggcacgatttcaaaatggaaaacacttgctaccgcggctgtttcgtatgaaagccagcgtgatgcgatcaaagcaaccttttcgacctcttttaacaacatcactatcgacaataaagccgtcactcctgtggtagatacacaggatttcgcaaaggtttcagttgcaggaaataacgttacgatctctgttaataaataa

SEQ ID NO: 7 - DNA, 570 bp, Bacillus subtilis
atgaactataacatcagaggagaaaatattgaagtgacacccgcgttaaaggatcatgtcgagaggaagatcggcaagctggagcgctattttgaccatagcgtggatgctgatgtgaacgtcaacttgaagttttacaatgacaaggagtctaaggttgaggttacgattccgatgacagatctggcgcttcggtccgaggtgcataacgaggatatgtacaacgcaattgatctcgcaacaaacaaactggaacgtcaaatccgtaagcataaaacgaaagtaaaccgtaaattccgtgagcagggctctccaaaatatttattggcaaacggtcttggctctgatacagatattgcggttcaggatgacatagaagaggaggagagcttggacatcgtccgtcagaaacgctttaatttaaagccgatggatagtgaagaagcgatcttgcaaatgaatatgctcggccataatttctttgttttcacaaatgcggaaacaaaccttacaaatgtcgtgtaccgcagaaatgacgggaaatatggcttaattgaaccgactgaataa

SEQ ID NO: 8 - DNA, 477 bp, Bacillus subtilis
atgactatatgtttcctattattttcttcttattactttagcaatatttcacctcagaatccactgttcaaaaaaaattttttgcaacaattgtctccccaaggctttggcttttatagtaaaagccctacagaagaaaacatttcatttcacacaaaagaaaatttaaagttacctaatgcacttcccaataatttttttgggataaaaagagaaggaagagttcaggcaatagaattaggcaaaattgtagagaatatcgatccaaagaattggaaaacttgtgaaaacaacaactcctgcacaaatttagagaaacaaataaagcctattaaggttataaaaaatgaagattatatacatcttagcaaaggagaatacctaatatatcgccaaaaaccactctcatggtattggatagactttaagcaaactacctcttttgaaagaaaggtgctaaaaataaaaatagtatga

SEQ ID NO: 9 - DNA, 972 bp, Bacillus subtilis
atgaagatattaaatagtttagaaggttatattgacacctataatccatggaaaaatacatatgcactttttagaagtttacttggtttctcaacattactagtactattattcaatagtactgatattttatttagttatagtgcaaataatgtcacatgtgaaaatgtctatatccctaccgctttttgttttgctaaagaatatagtatcaattttgagattataagatacttaatgatttttatattaaccttagtggttatagggtggagacctagatttaccggtttatttcactggtatatttgctatagtattcaaacttcagctttaactatcgatggtggagagcaaattgcaactgttctttcttttcttatattacctgttacattattagattcaaggcgaaatcattggaatataaagaaaaacaataatgaatctttcacaaagaagacagtattgttttatataatgacaataattaaaattcaagtttttatcatttatttaaacgcagctttagagcgattgaaaaataaagagtgggcagaaggaacagcaatttactatttcttttctgatccggtgtttggattacctgaatatcaacttaacttaatgaatccactacttgaaagcaattttattgttgtcatcacttggttagtaactatttttgagttgttcttagcagcaagcataatttcaaatatcagaataaagagaattgcccttgttttgggaatattatttcatattgggataatattcagcattggtattgtaagttttggcttgatcatgatatcagcattaattatatatctgcatcctgtacaacaaaatatcactatgaattggtgttctcctttatttaaatatatatatgtaaaaggaaagagaaatttcaaaagaataggaggtgaatcagtcaagtttcttacaaaattgtttcatagctaa

SEQ ID NO: 10 - DNA, 612 bp, Bacillus subtilis
ttgaaaagtaaattacttaggctattgattgtttccatggtaacgatattggttttttcattagtaggactctctaaggagtcaagtacatctgctaaagaaaaccatacattttctggagaagattactttagaggacttttatttggacaaggggaagttggtaaattaatttcaaacgatttggaccctaaactcgtaaaagaggcaaatagtacagaaggtaaaaagttagtaaatgatgtagtcaaatttataaaaaaagatcaaccacaatatatggatgaattgaaacaatcgattgacagcaaagaccctaaaaaactcattgaaaatatgaccaaagcagaccaacttatccaaaaatatgctaagaaaaatgaaaacgtaaaatactcttctaataaagttactccatcttgtgggctttatgccgtctgtgtagcagctggatatttatatgttgtgggcgttaacgcagttgcattacaaacggctgccgcagtaacaactgcagtgtggaaatacgttgccaaatattcctcttcagcttctaataattctgatttagaagcggctgctgcaaaaaccctaaaattgattcatcaataa

SEQ ID NO: 11 - DNA, 1041 bp, Bacillus subtilis
atgaaggcagcaagatggcataaccaaaaggatatccgtattgaacatatcgaagagccaaaaacggagccgggaaaagtaaagatcaaagtcaaatggtgcggcatctgcggaagtgatttacacgaatatctgggcggcccgatctttattccggttgacaaaccgcacccattaacaaatgaaacggcacctgtcacaatggggcatgaattctccggtgaagttgtcgaagtcggagaaggcgttgaaaattataaagttggagaccgcgttgtagtcgagccgatttttgctacacacggccaccaaggcgcctacaaccttgatgaacaaatgggattcctcggcttagccggcggaggcggcggtttctctgaatacgtctctgtggatgaagagcttttgttcaaacttcctgatgaattatcatatgaacaaggcgcgctcgttgaaccttctgcagttgctctatacgctgtccgctcaagcaaactcaaagcaggcgacaaagcggctgtattcggctgcggcccgatcggacttcttgtcattgaagcgctgaaggctgccggtgcaactgatatttacgctgttgagctttctcctgaacgccagcaaaaagctgaggagcttggcgcgatcatcgttgatccgtctaaaacagacgatgtagtcgctgagattgcagaacgtacaggaggcggtgttgacgtagcattcgaagtcactggtgtcccagtggtgttacgacaagccatccagtccactacaattgccggtgaaaccgtcatcgtcagcatttgggaaaaaggtgctgaaatccatccgaacgatatcgtaatcaaagaacgtacagtaaaaggaattatcggataccgcgacatcttcccggctgtattgtcattaatgaaagaaggctatttctcagccgacaaactcgtaacgaaaaaaatcgtactagatgatttgatcgaggaaggcttcggggctcttattaaagagaaaagccaagtcaaaatccttgttagacctaactaa

SEQ ID NO: 12 - DNA, 171 bp, Bacillus subtilis
atggaaaagctatttaaagaagttaaactagaggaactcgaaaaccaaaaaggtagtggattaggaaaagctcagtgtgctgcgttgtggctacaatgtgctagtggcggtacaattggttgtggtggcggagctgttgcttgtcaaaactatcgtcaattctgcagataa

SEQ ID NO: 13 - DNA, 414 bp, Bacillus subtilis
atgaaaaagtggattgttttatttcttgttttaatagcagcagccattagtattttcgtttatgtttctacaggtagcgaaaaacctttttataatgatataaatttaactcaatatcaaaaagaagtagactctaaaaaacctaaatttatttatgtttatgagacaagttgtcctccttgtcaagaaataaaacctgagttaaatgaagtaattaaaaaagaaaagttaaaagtacaggctttaaatattgaagaaaaggaaaattataacactgaatttttagataaatataatttgaataaaactccaacgattctctattacaaagatggcaaagaaaaagatcggttagagggctatagaagtgcaagccaaatagaaaagttctttgataaaaatggtgatagataa

SEQ ID NO: 14 - DNA, 1269 bp, Bacillus subtilis
atgaaactgagtgatatttatttggaattaaagaaaggctatgccgattctttattgtattcagatttgtcattgttggttaatataatggaatatgaaaaagatattgatgtgatgtcaattcaatctttggttgcaggttatgaaaaatcagatactcctacaataacatgcggtattatagtttataacgaaagcaagagaattaaaaagtgtttaaatagtgttaaagatgattttaacgagattattgttctagattcatactccactgatgataccgttgatattattaaatgtgattttcctgatgttgaaattaaatatgaaaagtggaagaatgatttttcctatgctagaaataaaattatagagtatgctacttccgaatggatttattttattgatgcagataatttatactctaaagaaaacaaagggaaaatagctaaagtagctagagttttagagtttttttctattgattgtgtagttagtccatatatagaagaatatactggacatctatattctgatacacgaagaatgtttcggctcaatggtaaagttaaatttcatgggaaagtgcatgaagaacctatgaattataatcatagtctaccttttaatttcattgtgaaccttaaggtttaccataatggatataatccttcagagaataatataaaatcaaaaacacgaaggaatataaatctcacagaagaaatgttaagattggagcccgaaaacccaaaatggttattctttttcggcagagaactacatttacttgataaagatgaagaagcaattgattatctgaaaaaatcaataaacaactataaaaaatttaatgatcaaagacattttatagatgctttagtgctattatgtactttattattgcagagaaataattatgttgacttaactttatatttggatatattggaaactgaatatccaagatgtgttgatgttgattactttagatctgcaattttgttagtagatatgcaaaataaacttacttctttaagcaatatgattgatgaagctcttacagacgagagatacagtgctataaatacaacaaaagatcactttaaaagaattttaataagccttaatattcaactcgaaaattgggaaagagtaaaagaaatatcaggggaaattaaaaatgataatatgaaaaaagaaattaaacaatatcttgccaactcactccacaatattgaacacgtcctgaaaggaattgaagtatga

SEQ ID NO: 15 - DNA, 447 bp, Bacillus subtilis
atgaatacaagatatgtaaaatcattttttttattactgttttttctctctttctttggcacaatggctagtttattctacagtgagatcatgcatttcaaaccatgtgttctatgttggtatcaaagaatatttctatatcctatacctattatcttactaataggcttattaaaaaaagatcttaattcgatattttatgttgttttcctttcatcaattggattgattattgcgttttatcattatattatccaacttacacaaagcaaaagtgtcgtatgtgaaattggaaccaacagctgcgcaaaaattgaagtagagtatctaggctttattacattacccttaatgagttcagtatgttttgcattgatatttggtataggactgaaattaattatcaaaagcaagaaattaaaacaaaatcaacatgtatataattga

SEQ ID NO: 16 - DNA, 954 bp, Bacillus subtilis
atgaaaaagatatctcttaccttattaatcttacttctcgcgctgacggcggcagcttgcggcagcaaaaatgaatcaactgccagcaaggcaagcggcacagcatctgagaagaagaaaattgaataccttgataaaacatatgaagtaactgtaccgacagacaaaattgccattacgggaagcgttgaatcaatggaagacgcgaaattgcttgacgttcatccgcaaggcgcaatttcattctccggcaaattccctgatatgttcaaagacatcactgataaagccgaaccaaccggagaaaaaatggagccaaatattgaaaagattcttgaaatgaagccagatgttatccttgcttcaacaaagtttccggaaaaaacgctgcaaaaaatcagcacagcaggcacgacgatcccagtttctcatatctcttcaaactggaaggaaaacatgatgcttcttgcccagctgactggaaaagagaaaaaagcaaagaaaattattgcagactatgaacaggatctaaaagaaataaaaacaaaaatcaacgataaagcgaaagattcaaaagcgcttgtcatcagaatcagacaaggcaacatttacatttaccctgaacaggtgtatttcaactccacactatacggtgatttaggccttaaggcgccgaacgaagtaaaggctgcaaaagcgcaagagctgagttcattagaaaaattaagtgaaatgaacccggaccatattttcgtccaattttctgatgatgaaaatgcagacaaacctgatgccttaaaagatttagagaaaaatccaatctggaaaagccttaaagcagtcaaagaagaccatgtgtatgtcaactcagtggaccctctcgcacaaggcggcacagcttggagcaaagtccgtttcctgaaagcggctgctgaaaaattgacacaaaactaa

SEQ ID NO: 17 - DNA, 1005 bp, Bacillus subtilis
atgtattcaaaacagtggacacgtatcatattgattacttctccatttgctatagcgctgtcacttttgctttcaatcctttatggggcaaagcatctcagcacagatattgtttttacatctcttattcatttcgatccgggaaacacagaccatcaaattatatggcattcccggattccaagggctgccggcgctctgctcataggggcagcccttgctgtttctggagcgcttatgcagggcattacgcgcaattatttagcttcgccatccattatgggtgtttcagatggttcagcgtttatcattacgctttgcatggttctgctcccgcaatcatcttcgattgaaatgatgatatactcttttatcggctcagcgttaggagcggtgttagtattcggccttgccgccatgatgccaaacggatttacccccgtgcagctcgccatcatcggcacagtcacaagcatgctgctcagcagcttatcagcggccatgtcgatttattttcaaatttctcaggatctcagtttctggtacagtgccagacttcatcaaatgagtccagatttcctgaagcttgccgctccgtttttcctgattggcattataatggccatttctctcagcaaaaaggtaaccgctgtatcattaggggacgacatttctaaaagcctggggcaaaagaaaaaaaccattaaaatcatggcgatgctttccgtcatcattctaaccggcagtgccgtagcgctggccggaaaaattgcgtttgtcgggttggttgttccgcatatcacgagatttctcgtcggctctgattacagcaggctgattccgtgttcctgtattttgggcggaatctttttaaccctgtgtgatctcgcaagcagatttatcaactatccgtttgaaacaccgattgaggtcgtaacatccattatcggcgtacctttcttcctttatttaattaaacgaaaaggaggggagcaaaatggctaa

SEQ ID NO: 18 - DNA, 1185 bp, Bacillus subtilis
atggctaaaaaatatgcattgttcatcgctttgattcttgttgtcagctatttcagcttaacgagcggatcattttctgttcgtcctgctgagctgctctccactctttttcaaatcgacccgaatccgcagtatgaaattttgctgttcgatttaagactgccgcgggttgtcatggctgctattattggactcggtcttggcattgcaggcgctgttatccaggccatcacgagaaacgggcttgctgaccctggaattctcggaatcaacgcaggggcaggagctggcattgtagcgtttatgctcttattccaaggccagaaggaagtgacatccatagctgcagcgatgggaatgccgctctttggattgataggcgggctcatcgcggcgatcctgatttacatatttgcatggcacagaggcaatttagattcaggaagaattattttggtagggattgcgatcaattcaggattcagcgccctgtctttgtttttatctttaaaaatggacccgcaagactatcaaatggccatggtgtggaaaaacggaagcatctggtctgccaactggacgtatattacagctgtactcccatggatgctgctgtttataccgattcttatcggcaaatcccgcctgctcgacaccattcgttttgatgaagacacagtcagaagcctcggtatttcatcaaataaagaaaaaaccatccttctcgttgcctgtgtagcaatcatcagcgcctgtgtctccgtagcgggaagtatggcgtttgtcggcttaattgctccccatatctcacggagactggctggcgtcgaacatcgctatatcctgccactgagcggtttaatcgggatgcttcttgtgataagcgcagactttgccggaaaactgttttttcagcccgcagaagtgcccgcagcatcattttggcgatcctcggagttccttatttcttatatctgcttttcaagcaaaaaaagggggagaatgcttgaaaggatctctttcagaacacaaagccggcaacaggaggttcacgctgtacctccctccttcctacagcacagacagcgggggatttcctgctgtttacgtgcaggatggcagttctttgttccaaaaccaaatcgaattactagaaagcgcctttcaacagcaaaggctccctga

SEQ ID NO: 19 - DNA, 477 bp, Bacillus subtilis
atggctaaagccttgattacatatgccagcatgtcaggaaatacagaagacattgccttcataataaaagatacgcttcaggaatatgagttggatatcgattgtgtcgagataaatgatatggatgcgtcttgtttaacctcctatgattatgtactgattggcacctatacatggggggacggcgatttgccctacgaagcggaggattttttcgaagaggtcaaacagattcagcttaatggtttaaaaacagcctgcttcgggtctggcgattattcttatccaaagttttgcgaagcggtgaatttgttcaatgtcatgctgcaagaggcgggagctgctgtttaccaggaaacactaaaaattgaattagcgcctgaaacagatgaagatgtggaaagctgccgagcgtttgcgagaggttttcttgcatgggcagattatatgaacaaggaaaaaatccatgtttcataa

SEQ ID NO: 20 - DNA, 894 bp, Bacillus subtilis
atgtttcataaaggggcaaccgctgttacggcatcggcgttttctggatattttgtggcggtacaaagagaaggcatttttcattactctttggagcagggctggagaaagctttttcgtttgaaaagtaagatacactgtatcagctacatagggccttacttatttggcgttggtgaaaagggaacagtcattcgttcggctgatgaagggaaaacctggacgatgtcgagctttccgacaaatgcaacagtgtgggcgattaccggcagaaacaacgggtttgtctgcgcccacggtaagcattgtatttatgtatcggatgattttggtgtctcatggcgcgtagccaaaccttttgccgaatttcataatccccctgttatccggtcgttatgccttcacgggggcaatctctttatcggcacgcaaatacacgaatattttggcggcatttgggcttacgacattaagcgtgacactgtccaagttgtcaaaaaagaaaaaaaccggatgacggcatccatgctcgtgttcaatgaaaattggctggtggcggcgatgggttctgtgaaaggaaagcacggtgctgtcactgtaaggaatcttttgaatggtgaagaattcaccatacaatccagtatgatcagaaatgaagaatcatttcttgatctttcagaggatgatggcattatatatgtcactacaacacaagatgaaaatggtttttcgagaatttaccaggttgatctcgaagcccggtcgttaaaatggttcgataccattaagggacatggatggagagtggccaatcagaaagagaatttcttttgcgcaggcttgtatgaatgtaaatttgtccagccgtacgaagtttcagcaatgattcattag

SEQ ID NO: 21 - DNA, 537 bp, Bacillus subtilis
ttggcgaagattttgctcgtttatgcaacaatgtcaggcaacactgaagctatggcagatttgattgaaaaggggcttcaggaggcgttagcagaagtagaccgtttcgaagcaatggatattgatgatgcccagctgtttaccgattatgaccatgtcataatgggaacctacacgtggggagacggagatctgcctgatgaatttttagatcttgttgaagacatggaggagattgatttttccggcaaaacatgcgctgtattcggttccggtgatacagcatatgaatttttctgcggagcggttgatacgctagaggcaaaaataaaagaacgcggtggagacattgtgctgccttcggtaaaaatcgaaaataatccagaaggtgaagaagaggaagaattaataaacttcgggagacaattcgcaaagaaaaagcgggtgcgctgtctgatcactcactgggaactgctaaaacggctgttcctttttttcttgtctttgtatctttcctttgatacagtaatgaggtag

SEQ ID NO: 22 - DNA, 786 bp, Bacillus subtilis
atgaatgcaaagggtatagagggaaaaattgcttttataacaggggctgcccaaggaataggcgaagctgttgcgcggacgcttgccagtcaaggcgcacatattgcggcagttgattataatcctgaaaagctggaaaaggttgtgagcagcctcaaagcagaagcccgccatgcagaagcttttcctgcggatgtgagagacagcgcggcgattgacgagatcacggcgcgcatcgaacgtgaaatggggccgattgatattttagtgaatgtagcgggtgtccttcgcccgggactgatccattcgcttagcgatgaggaatgggaggcgacgttctcagtgaattcgactggcgtatttaacgcctcgcgttcagtcagcaaatatatgatggaccgaagatcgggttcgattgtaacagtcggatcgaatcctgccggtgtaccaagaacatctatggcggcatatgcgtcttcaaaggctgcggctgtgatgtttacgaaatgccttggccttgagcttgcagaatacaatattcgctgcaacattgtatctcccggatcaacggaaacagacatgcagtggtcattatgggccgacgagaatggagcggagcaagtcataaaaggatcacttgagacatttaaaacagggatcccgctcaaaaaactagccaagccttcggatattgcggatgcggtgctctttttggtttctggccaggcagggcatattacgatgcataatttatgcgtagatggcggggcgaccttaggcgtgtaa

SEQ ID NO: 23 - DNA, 939 bp, Bacillus subtilis
atggctatacctgccattcagccgtatcaaatgccgacagcatctgatatgccgcaaaacaaagtatcatgggtgcctgatccgaatcgggctgtcttgttaatacacgatatgcaaaactattttgttgatgctttcacagcgggagcgtctccggtaacagagctttcagcgaatatacgaaagctgaagaatcaatgtgttcagcttgggattcctgttgtctataccgcacagccgggaagccaaaatccggatgaccgtgcgctgctgacagacttttggggcccgggattaaacagcggtccttatgaggagaaaattataaccgagctggcaccagaggatgatgatcttgtgctgacaaaatggagatacagcgcgtttaagagaacgaatctgcttgaaatgatgcgcaaagagggacgcgatcagctgatcattacaggaatttacgcccatatcggctgtcttgttacagcatgtgaagcatttatggaggatattaaagccttttttgtgggagatgcagttgctgatttttcattagaaaaacatcaaatggcgctggaatatgcggctggacgctgtgcgtttaccgtgatgactgacagtcttcttgatcagctgcagaatgcgccggcagacgttcaaaaaacgtcagcaaacactggcaaaaagaacgtgtttacatgtgagaatatccgtaaacaaattgctgagcttctacaagaaacaccggaagacatcacagatcaagaggatttgctcgatcgtggtcttgattcggtaaggatcatgacattggtggaacaatggcgccgtgaaggggcagaggtgactttcgtggaattggctgaacgcccaacgatcgaagaatggcagaaattgctcacaactcgcagccagcaagtgctgccaaacgcggattatttataa

SEQ ID NO: 24 - DNA, 1197 bp, Bacillus subtilis
atgttggatcaaaacgttataacagaaacaaaagcggagcatttgcttcatgaatatcagccgggcgcctttttcttagcgtctcctcatcgtgtactgttagcgaaaggcatatgtgaaattgtaccggaggcagacgggcaaaaccaaatggaaaccctttctggccgaattgcagaggcgttacgtcaggcaaaacaatcagggcaaagccggccgcttgttgtcggggccgttccttttgatcaagtaaaagcagcgcggctcgttgtacctgaagaagtgcgctggtcaggaccgcttcaatttgatcatgaggaaaaggaacagcaggctgggcatacataccacataaagcctgttcctgaacctgaggattataaaaatggtgttgaacaagggctggcacgcattgccgatggaacactcagcaaaatcgtcctgtccagatcgctgcatttgacatcgcctgaaccgattcagacggatgaattgcttcgccatctggctcagcataactcgcatggctacacgtttgccgcagacgtgtccagtcaggaggaaacgtctccccgcagaacattgctcggagcaagtccggagcttctcgtttcaaggatgggaacacaggtcgtttccaacccattagccggctcaagaccgcgcagtaatgatcctgttgaagaccagcgccgggcagctgaattgctttctcccgcaaaggatcttcatgagcacgcggttgtcgctgacgcggttgcggcagcgctgagacctttctgccggacgctggaggttccggagaagccttcactgatcaaaacggaaacgatgtggcacctgtccagcgtgattaagggagagctttccgacccgtctgtaaccgcacttgaattggcggcggcgctccacccgacgccagccgtctgcggaacaccgactgatcttgcaagagaagcgattctcagcattgaaccatttgaccgcggtttctttaccggcatggtcggatggtgtgacgatgccggtgacggagaatggatcgtgaccatccgttgtgcagaagcagaagaacgctcactccgcctgtatgctggagctggtgttgtggccggttcaaagcctgaggacgagcttcaggagacgtccgcaaagtttcggacaatgctgcgggcaatgggcgtggatcacatatga

SEQ ID NO: 25 - DNA, 1488 bp, Bacillus subtilis
atgagttctttaacgatgcaagtgacgaaaaggctggagacatttttacagggaacaaagaagctttatattgacggaaagtttgttccgagtgcctcaggggcaacctttgacactccaaacccggcgaccggcgaaaccttgatgacgctgtatgaagcccaggctgcggatgtggacaaagctgttaaagctgcccggaaagcctttgaccaaggtgaatggagaacaatgtctccagcttcgagaagcagactgatgtataagctggcagacttaatggaagagcataaaactgagcttgctcagcttgaaacacttgataatgggaaaccgatcaatgaaacgactaatggagatattccgctggctattgagcatatgcgctattacgccggctggtgtacaaaaataacaggacagacgattccggtttccggcgcttattttaattatacgcgtcatgagcctgtcggtgtcgtcggccagatcattccatggaatttcccgctcctgatggcgatgtggaaaatgggcgcggcacttgcaacaggctgtacaatcgtcctcaaaccggctgaacaaacaccgctttcagctctttatttggcagaattaattgaccaagccggtttccctgccggtgtaatcaacatcatcccaggattcggtgaagatgcgggagaagcgctgacgaaccacgaagcggttgataaaattgcctttaccggttccactgaaatcggaaagaaaatcatgtccaccgcagcgaaaagcattaagcgtgtgacattggagctgggcggaaaatcgcctaatattcttctgccggatgcgaatttaaaaaaagccatcccgggcgctttaaacggtgtgatgtttaaccagggccaagtctgctgtgcgggctcacgtgtcttcattcataaagaccaatatgatgaagttgttgatgaaatggcatcctatgctgagtcactccgccaaggagcgggacttcataaagatactcaaatcgggcctctcgtaagcaaggaacagcatgagcgcgttctttcctatattcaaaaaggaaaagatgaaggagcaaaagcagtgaccggcggaagctgtccttttgaagcaggatattttgtcgcaccgactgtgtttgcgaatgttgaagacgaaatgaccatcgcaaaagaagaaattttcggacccgtgctgactgcaattccgtacgaaacagtcgatgaagttattgaacgggcaaaccattcagaatatgggcttgcagccggactatggacagagaacgtcaagcaggctcactatatcgcggaccgacttcaagccggaaccgtttgggtcaactgctataatgtgtttgacgcggcgtctccatttggcggttataaacagtcaggactcggacgagaaatgggatcatatgccttggataattacacagaagtcaaaagtgtatgggtaaaccttgaagactaa

SEQ ID NO: 26 - DNA, 1146 bp, Bacillus subtilis
gtgacaggtgtcatatcttcttcttccatcggagaaaagattaacgaatggtatatgtacatacgccgattcagcatacccgatgcagaatatttgcgacgagaaatcaagcaagagctggatcaaatggaagaagatcaagaccttcatttgtactattcactgatggagtttcggcacaacctaatgcttgagtaccttgaaccgttagaaaaaatgaggattgaggaacagccgagactgtctgatctgctgcttgagattgataaaaaacaggctcgtttaactggtctgcttgagtactattttaacttcttcagaggcatgtacgagctggaccagcgggaatatctgtcggctattaaatttttcaaaaaggccgaaagcaagctgatattcgttaaggatcggatagagaaagctgagtttttctttaagatgtctgaatcttattactatatgaaacaaacgtatttttcaatggactatgcacggcaagcatatgaaatatacaaagaacatgaagcttataatataagattgctgcagtgtcattctttatttgccaccaattttttagatttaaaacagtatgaggatgccatctcacattttcaaaaagcttattctatggcagaagctgaaaagcagccccaattaatggggagaactttgtacaatatcgggctttgtaaaaacagccaaagccaatatgaggatgccataccttatttcaaaagagcaatagctgtttttgaagaatcaaatattcttccttccttacctcaagcgtattttttaattacacagatccattataaattaggaaaaatagataaagctcatgaatatcatagtaagggaatggcttattcacaaaaggccggagatgtaatatatttatcagagtttgaatttttgaaatctttatacttatcaggcccggatgaagaagcaattcaaggattttttgattttctcgaaagtaaaatgttgtatgctgatcttgaagatttcgctattgatgtggcaaaatattatcatgaacgtaaaaattttcaaaaagcttctgcttattttttgaaggtggaacaagtaaggcaacttattcaaggaggagtgagtttgtatgaaattgaagtctaa

SEQ ID NO: 27 - DNA, 1098 bp, Bacillus subtilis
atgaataagatcgcacccgcagaaatcgctagcatgctcaacgattggtaccttgccatcaagaaacatgaagttgaagaatcctcccgtttatttgaagaagtgaagcctttattggatgacatggaagaggatcaggaggtgcttgcctacttctccttattggaactgcgccacaaggttttgcttcacgaggcgagaggacagggctttcagcatgaggagccttctcatatgaatgctacgtctgacatgctgaaatattacttttttctgtttgaaggcatgtatgaggcctataaaaataattatgacattgccattgggctgtataaagatgcagagcagtatctcgacaacattcccgatccgattgaaaaagccgaatttcacctgaaggtcggtaagctctattataagctgggacaaaatattgtgtccctcaatcatacacggcaagcagtcaaaacattcagagaagagacagattataaaaagaagctggcttcagccctgattaccatgtcaggcaattttacagagatgagccagtttgaagaagctgaggcttatttggacgaagcaattcggatcacgagtgaattagaggatcatttttttgaagcccagcttttgcataacttcggccttctacatgcgcaaagcggcaaatcagaagaagcggtttcgaaattagaggaggctctacagaacgatgagtatgcccgctccgcctattattatcattctgcctacttgctgatacgagagctgtttaagatcaaaaagaaagaacaggccttatcttattaccaagacgtgaaggaaaaattgactgctgagccgaatagaatatgtgaggcaaaaatagacattttatatgccatttatgcagaagggggtcatgcggaaacgtttcacttatgcaaacaacatatggatgacttgttgtccgagaaagagtatgacagtgtaagagaactttccattttggctggcgaacggtatagggaacttgagctttacaaagaagctgcccactttttttatgaagcattacagattgaagaactgattaaacgaacggaggttatataa

SEQ ID NO: 28 - DNA, 1296 bp, Bacillus subtilis
ttgagtcaagccataccgtcttcgcgtgttggtgttaagattaatgaatggtataaaatgattcgccagttcagtgttccggatgctgagattctgaaagcggaggttgagcaggacattcagcagatggaagaagatcaggatttactgatctattattctctgatgtgttttcggcaccagctgatgcttgattatttggagccgggaaaaacatacgggaatcgccctacagtgacagagcttcttgaaacgatcgagacccctcagaaaaaactcacaggtcttttgaaatactactctttgtttttccgcggcatgtatgaatttgaccaaaaagaatatgtggaagcgatcggatattatcgcgaggcggagaaagaactgccgtttgtgtcagatgatattgagaaagcggaattccattttaaagtggcagaagcgtattatcacatgaagcaaacccatgtgtcgatgtatcatattcttcaagccttggacatttatcaaaaccatcctctatacagcattagaacgatacaaagcttgtttgtgatcgccggcaactatgatgatttcaaacattatgataaagcgctcccgcatttagaggcggcgctggaattggcaatggacattcaaaatgacaggtttatcgccatttctctattgaacattgcaaacagctatgacagatcaggagacgatcagatggctgtagaacatttccaaaaagcggcgaaagtaagcagagagaaagtgcctgatctgcttccgaaagtcttgtttggattaagctggacattatgtaaagcgggccaaacacagaaggcgtttcagttcatagaggaaggattagaccatatcacagcacgttctcacaaattttataaagaattgtttctgttcttgcaggccgtgtacaaggagactgttgatgaacgaaaaattcatgatcttttaagctatttcgaaaaaaagaacctgcacgcttacattgaagcatgtgcccggagtgctgccgctgtttttgaaagcagctgtcactttgaacaagcagctgcgttttatcggaaagtgctgaaagcccaagaagatattctaaaagggagagtgtttatatgcctattaagaaaaaaagtgatgatgtgtctggctgttactctagttttcggaagcatgtcgtttccaaccctgacaaactccggtggatttaaggaatcgacagatcgaaatacgacgtatatcgatcattccccttacaaacttagtgatcagaagaaagcccttagctag

SEQ ID NO: 29 - DNA, 1116 bp, Bacillus subtilis
atgagtaagatcgcttctgaagttgtcgctactacactgaatgactggtacattgctataaaaaaacaaaaggttgatgaatcaataaaatattattcagagataaagaaactttttgatgaaatggaagaagatcaagaagttcttgcgtattatagtctattagaagaaagacataaaatgttgctgcattcttcacgaggagagcctttacaaaagcacacctattttactgaagacaatcaaaacttcataacaaaaacaaatgataaattagaatacaacttttatttatttgaagcaatgtacgaggcatacaacaaaaactatgatcgagcaattaacctatatggattagctgagaaaaagcttgcagaaattccagatgaaattgaagcagctgaattttactctaaagtctcttacttatatactcttgttaaacaaagcattgtggcacaacattatataaaaaatgcaatttcaatatataagcgacaccctgattataaatgcaaactagctacatcaacaatgattgcagctgcaaactatgctgatatgaaacgatttgaggaagcagaacaatattacttagaagcaattgatattgcaaaagaaacaaaagatgaatttttaaaagctcaattatttcacaatcttagtatcgtttattctgattggaacaaacctgataaatgcattgaatctcttgaaaaagcaataggaaatgaatcttggttacattcgatttattatataaattctttattcatgatgattaaagaactctttaaaattgacgaaaaaatgaaagccattaatttttacaataaagcacaggaaagactcatattaatggagaataaagtatacgaagccaaaatcagcatcctgtataacctttattgtggggaattaaaaaataatttcaataattgtattagtaatattgagtttttaaaacagcaaaatgaacttgaaagtgtagatgaattgtcctacatagctgcaaaaaggtttgaatcaataggtgcttttgaagaagcaacgagctttttcaatgcgaaaatttgggctgaacagaaaatgaatcaggtggagggaatcttatga

SEQ ID NO: 30 - DNA, 1089 bp, Bacillus subtilis
atgctgaaaagaacgccgttatttgacctgtataaggaatatggaggaaaaacgattgatttcggaggctgggagcttcctgttcaattttcttctataaaaaaagaacacgaggctgtccgaactgcagccggtttgtttgatgtatctcatatgggagaagtcgaagtgtcagggaacgacagtctgtcttttttgcaaagattgatgacaaatgatgtttccgcgttaacgccaggccgtgctcaatatacagcgatgtgttacccggatggcggaaccgtcgatgatttgcttatctatcaaaaaggagagaaccgctatctgcttgtcattaatgcttctaatatagataaagacttggcttggatgaaagaacatgcagcaggtgatgtgcagattgacaatcagtcagatcaaatcgcgctcttggctgtacagggaccgaaagcagaagcgatcttaaaaaatctgacagatgcggatgtgtctgcattaaagccgtttgcgtttattgatgaagccgatatcagcggccgcaaagcacttatttcacgcactggctatacgggagaagacgggtatgaaatttactgccgcagtgatgatgctatgcatatttggaaaaaaatcatcgatgcaggggatgcatacggattgattccatgcggtctcggtgcacgtgatacactccggtttgaagcgaacgtcccgctctacggtcaggagctgacccgggatattacaccgattgaagcaggtataggctttgctgtaaagcacaaaaaggagtctgactttttcggtaagtcagtattgagtgaacaaaaagaaaacggagcgaagcgcaaacttgtcggtctcgaaatgattgaaaaagggataccgcggcacggatatgaggttttccaaaatggcaagtctgtcggaaaggtgacaaccggcacgcagtcaccgacattaggaaaaaacgtcggccttgccttaattgattcggaaacgagtgagatcgggactgttgtagatgtagagatacgcaaaaaattagtgaaagcaaaggttgtcaaaacaccattttataaacgctaa

SEQ ID NO: 31 - DNA, 1347 bp, Bacillus subtilis
atgaagcaccgttatttgcccgcaacagaaaaggataaacaggagatgcttgctactatcggcgtaagcagcatcgatgatttatttgctgatataccggaaaacgtcaaatataaaaaagagcatcaaatcaaaaaagcgaaatcagagacagaattaacaagagaactgacaaagctggcctctaaaaatcgtgataccgtacaatacgcttctttcttaggagcgggtgtatatgaccactatcagcctgtcattgtggatcatgtcatttcgcgctctgagttttataccgcatatacgccttatcagccagagatttcacaaggagagctccaggctatttttgaattccaaacgatgatctgtgaactgacaggcatggatatcgccaactcctcgatgtatgacggcggaacagccttggcagaagcagcaatgcttgcttcaggccacacgaaaaagaaaaaaattgttgtgtcaaaaaccgtgcatcctgaatcgcgagaggtgctgaaaacttacgcaaaaggtcagtatattgatgttgttgaagtacccgctgcggatggcgttacggatcttgatgcattgcgccaaaccgtttgcgagaacacagccgcagtgatcgttcagtacccgaattttttcggcaggatcgagccgctaaaggatattgagcctatcgctcatcaagggaaatccatgtttattgtttcagccaacccgctggcgctaggtcttctcactccgccgggcaagtttcagtctgatatcgtcgtcggtgatgcgcaccgtttcggcattccttcagcatacggcggcccgcattgcggcttttttgccgttactaaaaaattaatgagaaaggtgccgggccgtctcgtcggacaaacggaagacgaaaacggaaaaagaggctttgtgcttaccctgcaagccagggagcagcatatccgccgggataaagcaacatcaaatatatgctcgaaccaagctttaaatgcgctggcagcatcagtggccatgactgctctcggaaaaaacggcgtaaaagatatagcccgccaaaatctcttaaaagccaactatgcaaagcaagaagcaaaaaaagcaggccttactgttatgtttgacgggccgatgtttaatgaatttgtcatcaaactggatgagccggtgagagctgtgaacaagcgtttgctggcaaaaggcatgattggcggatatgatcttgggttgacgtatccagagctggactgccatatgctgattgctgtaacagagctgagaacaaaagaagaaattgacgcactcattcaggaattgggggatcgccatgagtaa

SEQ ID NO: 32 - DNA, 705 bp, Bacillus subtilis
atgaatgagaatatgagtttcaaagaattatatgcgattgtcagacacagattcgtgctgattctgctcatcacaatcggcgtcacccttattatgggttttgtgcaatttaaggtcatttcaccgacctaccaggcgtcgacacaggtgctggttcatgaatcagacggtgaagaaaactcgaatctcagtgacatccagcgaaatcttcagtatagcagcacgttccaatcgattatgaaaagcactgccttgatggaagaagttaaggcggaattgcacctatctgaatcggcttcctcgctgaaaggaaaagtggttaccagcagtgaaaatgaatcagaaataatcaacgttgccgttcaggatcacgatccggcgaaagcagctgagattgcgaacacgttagtgaacaagtttgaaaaagaagtagatgaaagaatgaatgtacaaggcgtacatatattatcagaggcgaaggcttcggaaagcccgatgatcaagccggccaggctgcgaaatatggtcatggcttttggcgctgctgtcatgggcggcattacactggcattttttctgcattttctcgatgatacatgcaaaagcgcacggcagctcagcgagagaaccggattgccatgcttaggctccgttcctgatgtccacaaagggcggaatcgcgggataaaacatttcggggagtga

SEQ ID NO: 33 - DNA, 684 bp, Bacillus subtilis
gtgatctttagaaaaaagaaagcaaggcgaggtttggctcaaatatctgttttacacaataaatcagttgttgcggaacaatatcgcaccattcggacaaacattgagttctcatctgtccagaccaacttgcgatctatcctcgtcacctcctctgtgcctggtgaaggtaaatcgttcagtgcagcgaatcttgcggctgtctttgcgcagcagcaggaaaagaaagtactgctggtggatgccgatttaagaaagccgaccatcaatcagacgtttcaggttgataatgtaaccgggctgacaaatgtgctggtcggcaatgcttcactcagtgagacggtgcaaaagacgccgatcgataacttatatgtactgacaagcgggccgaccccgccaaacccggcagaactgttgtcttcaaaagcaatgggagatttaatatctgagatctatgaacaattcagcctcgtcatctttgattcccctcctcttttggctgttgcagatgctcagattctagcaaatcagacagacggcagcgtgctcgtcgttttaagcggaaaaacaaaaaccgataccgttctgaaagcaaaagatgcactggaacaatccaatgcgaagctgttaggcgctcttttaaacaaaaagaaaatgaaaaaatcggaacactattcctactag

SEQ ID NO: 34 - DNA, 1797 bp, Bacillus subtilis
atgattattgcgctggatacttacctcgttttaaattcagttattgcaggatatcaatttttaaaagattcctatcaattttatgactccggagcattactgcttaccgctgtcagcttgctcctcagctatcatgtgtgtgctttcctgttcaatcagtataaacaggtgtggacatacaccgggcttggcgagctgattgtcctgcttaagggcattacgctttcagccgctgtgaccggcgtcattcagtatgctgtgtatcacacgatgttcttccgtctgttaaccgcgtgctgggtgcttcagcttttgtctattggagggacccgtattttatccagagtattaaacgaaagcatcaggaaaaaacgctgcgcctcgtcccgcgcgctgattatcggggcgggctcaggtgggactctgatggtcaggcagctgctttcgaaagatgaacctgatatcatacctgtcgcttttattgatgacgaccaaacgaagcataaattagaaattatggggctgcccgtaatcggcggaaaagaaagtatcatgcctgcggtgcaaaagctcaaaattaattatattattattgccattccttcactccgcacccatgagcttcaggtgttatataaagaatgtgtgcgaaccggagtaagcattaaaattatgcctcattttgatgaaatgctgcttggcacacgaactgccggacaaatcagagatgtaaaagctgaggacttgctcggcagaaagccggtaaccctcgacactagcgaaatttcgaaccgcatcaaaggaaaaacagttctcgtcacgggagcgggcggatcaatcggctcggaaatctgccgtcagatcagcgcgtttcagcctaaggaaatcattctgctcggccatggggaaaacagcattcattcgatttatacagagctgaacggacgattcggcaaacacattgtgttccatacggaaatcgctgatgtgcaggaccgcgataaaatgtttaccttgatgaaaaaatacgagccgcatgttgtctatcatgcagctgcccataagcatgtgcctttaatggaacacaatccagaagaggcggtcaaaaacaatattatcggaacaaaaaatgtcgcggaagcagccgatatgtcgggaactgagacattcgtgctgatttcatcggacaaagcggtgaacccagccaacgtaatgggggcgacaaaacgattcgcagagatgattattatgaatcttgggaaagtcagcagaaccaaatttgttgctgttcgcttcggcaatgtactcgggagccgcggcagcgtcattccaattttcaaaaaacagattgaaaaaggcggcccggtgacagtaacacatccggcaatgacccgctatttcatgacgattcccgaggcatcaaggcttgtgattcaggctggggcactggcgaaagggcgtcaaattttcgttctcgatatgggagagcccgtaaagattgtggatcttgccaaaaacctcattcatttgtccggctacacgactgagcaggttccaatcgaattcacaggcattcgtccgggcgaaaaaatgtatgaagaattgctgaacaaaaatgaagtccatgctgaacaaatctttccaaaaattcacatcggtaaagcggtggacggcgattggccggtgctgatgcgctttatcgaggattttcatgagctgccggaagccgacctgagagcgaggctgtttgcggcaatcaatacatcagaagaaatgacggctgccagcgttcattag

SEQ ID NO: 35 - DNA, 1146 bp, Bacillus subtilis
atgacgaaaaagatattgttttgcgcgactgttgattatcattttaaggcctttcacctcccttattttaaatggttcaagcaaatgggctgggaggttcatgtcgccgcgaacggacaaaccaagctgccgtatgtggatgagaaattctccatcccgattcgcaggtcaccttttgaccctcagaacctggccgtttataggcagctgaagaaagtgattgacacttatgaatacgacattgtccattgccatacaccggtcggcggcgttctcgccagactggcggcgaggcaggcacggcggcacggaacaaaggtgctgtacacagcgcacggatttcacttctgcaaaggggcaccgatgaaaaattggcttctttactatccggttgagaaatggctttcagcatatacagactgcctgattacgattaatgaagaggattacatacgggcgaaaggacttcaaaggccgggcggaaggacgcagaaaattcacggcattggcgtcaataccgagcgtttccggcctgtcagtccgatagagcagcaaagactcagagaaaagcacgggttccgtgaagatgattttatattggtttatccggctgagctcaatctgaacaaaaaccagaagcagttaattgaagccgcagccttgctaaaagaaaaaattccctcactccgccttgtgtttgccggggaaggggcaatggaacatacgtatcaaacgttagctgaaaagcttggtgcctccgcccatgtctgtttttacggcttttgcagcgacatacatgagttgattcagcttgcggatgtatctgtcgcatccagcattagagaaggcctcggtatgaatgtgcttgagggaatggcggcagaacaaccggcgatcgccacagataatcgcgggcatcgggaaatcatccgcgacggagaaaacggttttctgatcaaaatcggtgacagtgctgcttttgcccgccggattgaacagctttaccataagccggagctctgccgaaagctgggacaggaaggccgaaaaacagccttgcgcttctcggaggcgcgaacggtggaagaaatggcagatatttattccgcgtacatggatatggatacaaaggagaaaagcgtatga

SEQ ID NO: 36 - DNA, 837 bp, Bacillus subtilis
atgaactcaggaccgaaagtttctgtcattatgggcatttataattgcgaacgcactttggcagaaagcatagaatccatactcagccaatcctataaaaattgggagctgattttgtgcgatgatgcgtcaacagacggcacgctccgtatcgcgaagcagtatgccgctcattacagcgaccgcatcaaactgattcaaaacaaaacaaataagcggcttgccgcatcattaaatcattgtctttcgcatgcgacaggcgattatatcgaacgtcaggacggagatgacctttcgtttccgcgccgtctggaaaagcaggtcgcgtttttagaaaagcaccgacactatcaggtggttggcaccggcatgcttgtgtttgatgaatttggcgtaagaggcgcccgcattctgccttctgttccggagccgggcatcatggcaaaagggactccattttgccacggcacgattatgatgagagcgagtgcctaccgcacgctgaaaggctaccggtcggtgcggcggacgagacgaatggaagatattgatttgtggcttcgcttttttgaagagggcttcaggggctataatcttcaggaagccttgtataaagtgagggaagacagcgatgcattcaaacggcggtcatttacgtattcaatcgacaatgccattcttgtctatcaggcgtgcagacgcttgaagcttcctttatctgattacatatatatcgcaaaaccgttaattcgcgcctttatgcctgcagctgtgatgaatcgctaccataaaaaaagagtgatgaaccaaaaggaagggcttgtcaagcatgaatag

SEQ ID NO: 37 - DNA, 1155 bp, Bacillus subtilis
atgaatagcagccaaaagcgcgtgctccatgttctcagcggcatgaacaggggcggcgcggaaaccatggtaatgaatttatatcggaagatggacaaaagcaaagtgcaatttgattttttaacgtatcgaaatgatccgtgcgcttatgatgaagagattttatctttaggcgggcggcttttttatgtcccgagcattgggcaaagcaatccccttacatttgtgaggaatgtgagaaacgcgataaaagaaaatgggccgttcagcgccgttcatgcgcacacggatttccaaacgggttttatcgcccttgcggcaaggctcgccggagtgccggtcagggtatgccactcccacaatacgtcttggaagaccggcttcaactggaaggatcgattgcagctgctcgtgttcaggcggctcattttggcaaatgcgacagcgctgtgtgcctgcggagaggatgcgggcaggtttttatttggacagtccaatatggagcgggagcgtgttcaccttcttcctaacgggattgaccttgagttgttcgccccaaatgggcaggcggctgatgaagaaaaagcagcacgcggcattgcagccgaccggctcatcattggccatgtggcccggtttcatgaagtgaaaaaccacgcgttcctgttgaagcttgccgcacatctcaaggaaagaggcattcgctttcagctcgttctggcgggagacgggccgttgtgcggggagatagaggaggaggcgcggcagcagaatttgctatcagacgtcctctttttaggcacggaagaacggatccatgaactgatgcgaacattcgatgtatttgtcatgccgtctctgtacgaaggcttgccggttgtgcttgtggaagcgcaggcgtcggggcttccatgcatcatttcagacagcattacagaaaaagtcgacgccggtctcgggcttgtcacaagattaagtctttctgagccgatcagcgtctgggctgaaaccattgcaagggcggccgccgcaggcaggccgaagcgtgagttcatcaaagaaacactcgctcaacttggctacgatgcacagcaaaatgtaggagcgctgctgaatgtatacaacatcagcacggaaaaggaccataaccgatga

SEQ ID NO: 38 - DNA, 1104 bp, Bacillus subtilis
atgattgtatatgccgtcaatatggggattgtatttatttggtcttggttcgctaaaatgtgcggcggccgtgatgattcgcttgccacggggtatcggccgaataagcttttgatctggattccgctcgcttcacttgtgctcgtgtcaggtctccgctatcgagtcggcacggattttcagacgtacacgctgttgtacgaattggcgggcgattatcaaaatgtgtggcagatattcggtttcggcacagcgaaaacagcgacagatccggggtttaccgcactcctttggctgatgaatttcatcacggaagatcctcaaatcatgtattttacggtggcggtcgtgacctacagctttattatgaagacactcgccgactatggcaggccgtttgagctgagtgtctttttatttttgggaacctttcattattacgcatcttttaacggcatcaggcaatacatggtggcagctgttttgttttgggcgatccgttatatcattagcgggaactggaagcgatatttcctgattgtgctggtcagctcgctctttcattcgtcggcgctgattatgattccagtgtactttattgtcagaagaaaagcctggtcaccggcgatattcggcctatccgctttatttctcggcatgacatttttatatcaaaaatttatttctgtgtttgtcgttgtacttgaaaacagctcatacagccattatgaaaaatggctcatgacgaacacaaatggaatgaatgtgatcaaaatcgctgttttggttctgccgctgttccttgcattttgctataaagaacgactgcggagtctgtggccgcaaattgatattgtcgtcaatttgtgcctgctaggttttttgttcggccttttggccacaaaggacgtgatttttgccagattcaatatttatttcggtctgtatcaaatgatcctagtcccttatttcgtcaggatatttgatgaaaaatcgaacgctcttatctatatcgctatcgttgtttgttattttctttacagttatttgcttatgccggtcgattcatcggttctgccttacagaacgattttttcccggtaa

SEQ ID NO: 39 - DNA, 1077 bp, Bacillus subtilis
atgtcgttacaatcgttgaaaatcaattttgcagaatggctgctgctaaaggtcaaatacccgtcccaatattggctgggagcggcagatcaaccggtaaaggccgcagcacatcagaaaaaaatcatactgaccctgctgccgtcccatgacaatttgggagatcacgcaattgcttatgccagcaaggcatttcttgagcaagaatacccggactttgacatcgtcgaggtcgatatgaaggacatttacaaatcagcaaaaagcctgatccgctcgcgccatccggaggatatggtctttatcatcggcggcggaaacatgggggatttataccgttatgaggagtggacgcgccgcttcatcattaaaacattccatgactatcgggttgtccagcttccggcaacggctcatttttctgacacgaaaaaagggcgcaaagagctgaaacgggcacagaaaatttataatgcgcaccccggcctattgctgatggcgcgggatgaaacaacgtatcaatttatgaaacagcattttcaagaaaaaacaattttgaagcagccggacatggtgctgtatttagacagaagcaaggctcccgcagaacgcgaaggggtttatatgtgtttgcgcgaggatcaggaaagcgtgcttcaggaggagcagaggaaccgggtcaaggctgcgctatgtgaggaattcggcgagatcaaatcctttacgacaacgatcggccgccgggtcagccgcgatacacgcgaacatgaacttgaagcactgtggtctaagctgcaaagcgcagaagccgtcgtcactgacaggcttcatggcatgattttttgcgcgctgacaggaacgccgtgtgttgtcattcgctcctttgaccataaggtgatggagggctatcaatggcttaaagacatcccgttcatgaagctgatagaacatccggagccagagcgcgtaacagccgcagtcaatgagcttttaacaaaagaaacatcccgtgcaggctttccgagagatgtgtattttaaaggtctgcgtgacaaaatcagcggtgaagcgcaatga

SEQ ID NO: 40 - DNA, 1035 bp, Bacillus subtilis
atgatcccgctcgtcagcattattgtcccgatgtataatgttgaaccatttatagaagagtgcattgattctttgcttcgtcaaacgctttctgatattgaaatcatcctcgtgaatgacggaacaccggatcgttcaggcgaaattgcagaggactatgcaaaacgggatgcgagaatccgggtcattcatcaggcaaacggcgggcttagttcagcgcgaaatacgggaataaaggccgcgcggggcacttacatcggctttgtagacggagacgattatgtatcatccgccatgttccagaggctgactgaagaagcggagcaaaatcagcttgacatcgtcggatgcggtttttacaagcagtcatcggacaggcggacatatgtgccgccgcagcttgaggcaaaccgcgtgctgacgaaaccagaaatgactgaacagcttaaacatgctcacgaaacgagatttatctggtatgtatggcgttatctttaccgtcgtgagctttttgaaagggcgaatctgctgtttgatgaagacatccgttttgctgaagactctccctttaatttgtccgcttttcgcgaagcggagcgggtgaaaatgcttgatgaaggattgtacatttatcgtgaaaacccgaacagcctgacagaaatcccttataagccggcgatggatgaacatcttcaaaaacaatatcaggctaaaatcgcattctacaatcactacggcttagcaggcgcatgtaaagaagatttgaatgtgtacatttgcaggcaccagcttccgatgcttttggcaaatgcctgtgcttctccgaattcgccgaaagacatcaaaaagaagatcagacagattttatcctatgacatggtgcggcaggctgtcagacatacaccgtttcagcatgagaaattattaagaggagagcgtttggtattagcactgtgtaaatggcggctcacttttctcatcaagctgtttttcgagcagcgggggacaatgaaaggcagtgcgaagcaggcatga

SEQ ID NO: 41 - DNA, 1002 bp, Bacillus subtilis
atgaaattgttaaaacgagaaggcttgtcattaactgaggaaaaagcgctgtggatgtaccaaaagatgctggagatcaggggctttgaagacaaagtgcatgaactgttcgcccagggagtgcttcccggattcgttcatttatatgccggtgaggaagccgtggctgtaggggtgtgcgctcatttacatgatggcgacagcattacaagcacccacaggggacatggacattgtatcgccaaaggctgtgacctggacggcatgatggcggaaattttcgggaaagcgaccggattgtgcaaaggcaagggcggttctatgcacattgcggatcttgataaaggcatgttaggcgcaaatggaatcgtcgggggcggctttacgctcgcatgcggatcagcgctcacggctaaatataaacagactaaaaatgtaagcgtttgctttttcggggacggggcaaataaccaaggtaccttccacgaagggctgaatttagcggctgtatggaaccttcctgtcgtatttgttgctgaaaacaacggctatggcgaagctaccccatttgagtacgcatcagcctgtgattcaatcgccgatcgggcggctgcttataacatgccgggggttacagttgacggcaaagatattttagcagtttaccaggcagccgaggaagcgatagaaagagcaagaaacggcggcggcccgtctttgattgaatgtatgacctacagaaactacggccatttcgaaggagatgcccaaacctataaaacgaaggatgaaagagttgagcaccttgaagaaaaagatgccattcaaggttttaaaaactaccttttaaaagaaacagatgctaataagctgtcagacattgaacagcgtgtcagcgaatcgattgaaaaagccgtctcgttcagcgaagacagcccatatccaaaagattcggagctgctgacagatgtgtatgtgtcatatgaaaaaggaggaatgtaa

SEQ ID NO: 42 - DNA, 1029 bp, Bacillus subtilis
atggcgagagtcataagcatgtcagacgcgatcaatgaagcaatgaagcttgcgatgagaaaagacgaaaatgtgcttttgatcggtgaggatgtcgccgggggagcggcggtcgatcatttgcaggatgatgaagcatggggcggtgtattaggggtcacaaagggactcgtacaggaattcgggcgtacaagagtgctggacactccgatttctgaggcaggctatatgggagcggctatggctgcggcatcaaccggtttgagaccgattgccgagctgatgtttaacgattttatcggcacgtgctttgatcaggtgatcaaccaaggggcgaaattccgttatatgttcggcggaaaagcgcaagtgccgattaccgtccgcaccacatacggagcagggttccgggccgctgcccagcattcacaatcgctgtatggccttttcacgagcatccctggactgaagacagttgttccatccaatccgtatgatgccaaaggtcttttgcttgcagcaatagaagataatgatccggtgtttttctttgaagacaaaacgtcctacaacatgaagggcgaggtgccggaagattattatacaattcccctcggaaaagcggatatcaaacgcgaaggcaatgatgttacgctctttgcagtcggcaagcaggtcaatactgcgcttgaagcggctgcacagctttcagagaggggcatcgaagccgaggtccttgatccccgcagtctgtctcctctggatgaggatgcgattttcacatcgttagaaaaaacaaaccggctgatcatcattgatgaagccaatccgcgatgcagcattgccacggatattgctgcgcttgtcgctgacaagggctttgatttgcttgatgcgccgattaaacggattacagcgccgcatacaccggttccgttttcaccagtgcttgaagatcaatatttgccgacaccagataaaattgtcagcgtcacgcttgaattgcttggcgagccggcattgaattaa

SEQ ID NO: 43 - DNA, 1197 bp, Bacillus subtilis
atggcggtaaaagtagtgatgccaaaattgggaatggccatgaaacaaggggaagtatcgatatggaataaaaaagtaggcgacccggttgaaaagggagaaagcattgccagcattcaatcggagaaaattgaaatggagatcgaagcgcctgaaaaaggaacgctgatcgatatcaaagtgaaagagggagaagaggttccgcccggcacagctatctgctatatcggggacgccaatgagtcggtgcaggaagaggcgggggcgcctgttgctgaagacaatatgccgcaagccgtccagcccgtcaaacaagaaaacaaacccgcagcctccaaaaaagatcgaatgaaaatatctccagtcgccaggaaaatagcagaaaaagcaggattagacctaaaacaactgaaaggaactggaccaggcggacgaatcgtgaaggatgacgtaacaaaggctcttgctgaacagaaaaaagatcaagcaaagcctgtttcggagcagaaagcgcaggaaatcccggtgacaggcatgagaaaggtcatcgctgcccgaatgcaggaaagcctggcaaacagcgcgcagctgacgatcacgatgaaagctgatatcaccaagcttgccactcttcaaaaacagctttcaccaactgcggaagagagatacggcacaaaactgacgatcactcattttgtctcaagagccgccgttctcgctctgcaagctcaccctgtgctgaacagcttttatcaaaatgagcgcatcatcacacatccccatgtgcaccttggtatggctgtagccttggaaaatggcttagtggtgcctgtcatccgccatgctgaaaagctatcgctgattgaactggctcaatccatctcagaaaatgccaaaaaagcacgcgagggacgtgcgggaagcgaagaactgcaaggatctactttctccattacaaaccttggcgcgtttggagttgagcatttcacaccgatactaaatccgccggaaacaggcattctcggcatcggagcaagctatgacacaccggtgtatcaaggggaggagatcgtcagaagcacgatcctgccactcagcctgacatttgatcacagagcgtgtgacggcgcccctgccgctgcattcctgaaggcgatgaaaacatatttggaagaacccgcagcattaattttatag

SEQ ID NO: 44 - DNA, 1377 bp, Bacillus subtilis
atgacattagccattatcggcggcggacctgcaggctatgcggctgcggtttccgcggcacagcagggcagaaacgtgctgctcattgacaaaggcaagcttggggggacctgcctgaatgaaggctgcatcccgacaaagtctttgttagaaagcgcaaacgttcttgataaaatcaagcatgccgacagctttggaatcgaacttccggcaggtgcgatatcagtcgattggagtaaaatgcaaagccgaaaacaacaggttgtcagtcagcttgtccaaggcgttcagtacctaatgaagaaaaatcaaatacaggttgtaaagggaacagcctcctttctttctgaaagaaagctcttgatcgaaggagaaaacggaaaagaaatcagagaggcggaccaagtattgattgcctccgggtcagagccaatcgagctgccttttgccccatttgacggcgaatggatcctcgacagcaaagacgcgctttctctttccgagattccgtcttcactagtcattgtcggcggcggtgtcatcgggtgtgagtatgcagggctgttcgccagattgggatcgcaggtgaccatcattgaaacagcggaccggctgatcccggctgaagatgaagatattgcccgtctctttcaggagaaacttgaggaagacggtgtcgaagtgcatacttcatccagattagggcgggtggatcaaacggccaaaacggcaatatggaaaagcggtcagcgagagtttaaaacgaaggccgattatgtgctggtggcgatcggcagaaaaccccgtcttgacggattgcagctggaacaggccggagttgatttttctccaaagggcattccggtgaatgggcacatgcagacgaacgtgcctcatatttacgcgtgcggagatgctatagggggcattcagctcgcgcatgccgctttccatgagggcatcatcgctgcttctcatgcttccggaagggatgtcaaaatcaatgagaaacatgtgccgcgctgcatctatacgtccccggaaatcgcgtgtatcggaatgacagaacgacaggcaagaagcatatacggggatgtgaagatcggcgaattttcattttccgcaaacggcaaggcgctcattaaacagcaagcggaaggaaaggtcaaaatcatggctgaaccggaattcggcgaaatcgtgggtgtctcgatgattggcccggatgtaaccgagctcatcggccaagcggcagcgatcatgaatggtgagatgacggcagatatggcggagcattttatcgccgcccatccgactttatcggaaacattgcatgaggcgctgttaagcacgatcggccttgcggtacatgcataa

SEQ ID NO: 45 - DNA, 582 bp, Bacillus subtilis
atgacagccgtttgtttagtaagacatggagaaaccgattggaacctgcagcaaaaatgccaaggcaaaaccgatatcccgctaaacgcaacaggtgaacgccaagcaagagaaaccggagaatatgtaaaggacttttcttgggatattattgtgacgagcccgctgaaaagagcgaaaagaaccgcggaaattattaatgaatatctgcatcttccgatagtcgagatggatgattttaaggaacgcgattacggcgacgcggagggcatgccgctggaggaacggacaaagcgctatccagataacatctatccgaatatggaaaccttagaagaactcactgacaggctgatgggcggtttggcaaaagtgaatcaggcgtatccaaacaagaaggtgctgatcgtggcgcacggtgcggcaattcacgccctgctgacagaaatatccggcggtgacccggagcttcaaagcacccgtctcgtcaacgcctgcctcagcaacattgaatttgcagaagaaaaatggcggataaaagactataatatcaacagccacttatccggctttatcaaataa

SEQ ID NO: 46 - DNA, 1095 bp, Bacillus subtilis
atgaatgcggttattgttgatgcaaaacgaacgatctttggaaatcaaaacggactgctgaagcccttcctgccggaggatttggcggctcccatcatccgctgtctcagccgaaagctagaggatcaagttgacgaggtcattctcggaaacgctactggcagaggcggcaacctggccagactgtcagcccttcaagccggactgcctttatcggttcccggaatgacaattgacagacagtgcggctccggccttgaagctgtgcgctatgcctgcagccttattcaagcgggagccggcacgatgtatatcgcgggcggctcagaaagcagcagccaatcccctttttcagaacgggctcgcttttctccagatgcgatcggcgatccagacatgggcattgcggcagaatatacggctgcacgctattccatcagcagaagcatgcaggatgagtacgcgcttctcagccatcaacgcagcaggaacgcgcatgatgaaggattttaccgtgaagaagttgttgctctcggggaattggagacggacgaagcatttttgaaaacgcggccaatagaagcgattattccccgtgcaaagccggttttcgacaccagctccggaacagtcacagcagccaacagcagtggcatagcagacggagcagccgctcttttggtaatggaagaagaaaaagcagcagccctgggacttaagcctgtgcttcggtttatcggcagcgctgtcagcggcattcaccccaactttccgcccgcggcaccggttgtcgcgattcgtcagctcttacatacacacgatgtaacacctgatgatatcgatttatttgaaatcaatgaagcctttgccgtcaaaatttgtgtctgctcgcaagaactcggcattcccttttcaaaaatcaatgtgcgcggcggcgccttagctcttggccatccgtacggtgcatcaggtgcagctctggtaaccagattgttttatgaagcgaaaagacggccagactgtcaatatgctgttgcagccatcggaagcggcggcggaatcggactggctttattatttgaagttcttgcatag

SEQ ID NO: 47 - DNA, 1440 bp, Bacillus subtilis
atgacaattactcatacctattcatctactgccgaaacatcgcccggccgtgtagcgatccagactgaatcggagcaaatcacgtaccatgattgggatcggcttgtctctcaaaccgcaaattggctgcggtcacagccgagcatgccgaatcgtgtggcgatcctgctcccaaatagtctcgcgtttttacagctgtttgccggagccgcagcggctggatgtacggccattcccatcgacacacgctggagcccggctgaatgtaaggagcggctgtccataagcaatgcggatcttgtggttactttagcctttttcaaaaacaaactgacagatagccagacacctgttgtattgctggataactgtatggcagatatttctgaggcagccgctgatcccttgcctaccattgatccggagcaccctttttatatgggatttacgtcgggctcgacaggaaaaccgaaggcctttacgcgatctcaccgctcatggatggagagctttacctgtacagaaacagatttttcgatttcatcagatgataaggttctgattcccggagcgttaatgtcctctcacttcctatatggggctgtcagcactttgtttctcggaggaaccgtttgtttgctgaaaaagttttctcctgccaaagcgaaggaatggctgtgccgtgaatccatcagtgttctctataccgtaccgacgatgacagacgccctcgcaaggattgagggttttcccgacagtcccgtcaaaatcatttcatccggcgcagactggccggcagaatccaagaagaagcttgccgctgcatggcctcatctcaagctgtacgatttttacggcacatcagagcttagttttgtgacgttttcttcaccggaagacagcaaacggaagccgcattcagcgggccgcccttttcataatgtccggatcgaaatccgcaacgctggaggagaacgctgccagccaggagaaatcggaaaaatatttgtcaaaagcccgatgaggttttccggctatgtgaacggcagcacaccagatgaatggatgacagtagatgatatgggctacgttgatgaagagggctttctatacatatcaggaagagaaaacgggatgatcgtgtacggaggattaaatattttcccagaagaaattgaacgtgtgcttctcgcctgcccagaggttgaaagcgcggctgtcgttggcattcccgacgagtattggggagaaatcgctgtagctgtcattcttggaaacgctaatgccagaacactgaaagcctggtgtaaacagaaattagcctcctataaaattccgaaaaaatgggtgtttgcagacagcttgccggaaacgagcagcggaaaaattgcccgttccagagtgaaaaaatggctggaagagagtgtacagtataaatga

SEQ ID NO: 48 - DNA, 561 bp, Bacillus subtilis
atgctgaaattaatcgacatgatgcatattgcgattttcaccgcgctgatggcagtgctcggctttatgccccctctcttcttatcctttacacccgttccgattacattgcaaacgcttggtgtcatgttggcaggcagcattctcaggccaaagtctgctttcttaagccagcttgtctttttgctgctcgtcgccttcggagcgccgctgttgcccggcggacgaggcggttttggtgtgtttttcggaccgagcgcaggctttttgattgcttatcccctcgcttcatggctgatcagtttagccgctaacaggctgcggaaggtgacagtattgcgtctctttttcactcatatcgtattcggcatcatctttatttatctgcttggtataccggtacaagcttttatcatgcatattgatttgtcacaggccgccttcatgagccttgcatatgtgcctggtgatttgataaaagcggctgtatctgcatttctggcgataaaaatcactcaagccttgtctctttctgatacgatgtttacaaaaggaggatga

SEQ ID NO: 49 - DNA, 1299 bp, Bacillus subtilis
ttgttatttaaaaaagacagaaaacaagaaacagcttacttttcagattcaaacggacaacaaaaaaaccgcattcagctcacaaacaaacatgcagatgtcaaaaaacagctcaaaatggtcaggttgggagatgctgagctttatgtgttagagcagcttcagccactcattcaagaaaatatcgtaaatatcgtcgatgcgttttataaaaaccttgaccatgaaagctcattgatggatatcattaatgatcacagctcagttgaccgcttaaaacaaacgttaaaacggcatattcaggaaatgtttgcaggcgttatcgatgatgaatttattgaaaagcgtaaccgaatcgcctccatccatttaagaatcggccttttgccaaaatggtatatgggtgcgtttcaagagctccttttgtcaatgattgacatttatgaagcgtccattacaaatcagcaagaactgctaaaagccattaaagcaacaacaaaaatcttgaacttagaacagcagcttgtccttgaagcgtttcaaagcgagtacaaccagacccgtgatgaacaagaagaaaagaaaaaccttcttcatcagaaaattcaagaaacctctggatcgattgccaatctgttttcagaaacaagcagatcagttcaagagcttgtggacaaatctgaaggcatttctcaagcatccaaagccggcactgtaacatccagcactgttgaagaaaagtcgatcggcggaaaaaaagagctagaagtccagcaaaaacagatgaacaaaattgacacaagccttgtccaaatcgaaaaagaaatggtcaagctggatgaaatcgcgcagcaaattgaaaaaatcttcggcatcgtcacaggcatagctgaacaaacaaaccttctctcgctcaatgcatctattgaatccgcccgcgccggagaacacggcaaaggctttgctgtcgtggcaaatgaagtgcggaagctttctgaggatacgaaaaaaaccgtctctactgtttctgagcttgtgaacaatacgaatacacaaatcaacattgtatccaagcatatcaaagacgtgaatgagctagtcagcgaaagtaaagaaaaaatgacgcaaattaaccgcttattcgatgaaatcgtccacagcatgaaaatcagcaaagagcaatcaggcaaaatcgacgtcgatctgcaagcctttcttggagggcttcaggaagtcagccgcgccgtttcccatgtggccgcttccgttgattcgcttgtcatcctgacagaagaataa

SEQ ID NO: 50 - DNA, 1350 bp, Bacillus subtilis
atgaagaaaaaatcattctcaatcgtaatagcgggcggagggagcactttcactccagggatcgtactcatgctcttggaccatttggaggagtttccgatcagaaagctgaagctgtatgataatgataaggagagacaggatcgaattgcaggcgcctgtgacgtttttatcagagaaaaagcgccggatattgaatttgcagcgacgactgacccggaagaagcttttacagatgtcgattttgttatggcgcacatcagagtagggaaatacgcgatgcgcgcgcttgatgagcaaattccgttaaagtacggagttgtcggccaggagacgtgcgggccgggcgggatcgcatacggtatgcgttcgatcggcggtgtgcttgaaatattagattacatggaaaaatactcgcctgatgcgtggatgctcaattattccaatccggcggcaattgtggctgaagctacgagacgccttagaccgaattctaaaattctcaatatctgtgatatgccggttgggatcgaagaccggatggcgcaaattcttggcttatcctcaagaaaagaaatgaaggtccgctattacggattgaatcatttcggctggtggacatcgattcaggatcaagagggcaacgatttaatgccgaagctgaaggaacatgtatcccaatacggttatattccgaaaacagaggctgaagctgtggaggcaagctggaatgacacgttcgccaaagcgcgtgacgtgcaggctgcagatcctgacacactgccgaatacgtatttgcaatattatttgttcccagatgatatggtgaaaaaatcaaatccgaatcatacgcgggcgaatgaagtcatggaagggcgcgaagcttttattttcagccaatgtgacatgatcacacgtgaacagtcctcggaaaacagcgaaattaaaatcgatgaccacgcatcgtatatcgttgatcttgcccgggcgattgcctacaacacaggtgaaagaatgctgttgattgttgaaaataacggtgcaattgcgaactttgacccgactgcgatggttgaggtgccatgcatcgtcggctcaaatggacctgaaccgattaccgttggcaccattccgcaattccagaaagggctcatggagcagcaggtatccgttgagaagctgactgttgaagcgtgggcagagaaatcgttccaaaagctgtggcaggcgctgatcctgtcaaaaacagtgccgaacgcgcgtgtggcaagactcattcttgaggatttagtggaggccaacaaagacttctggcctgagcttgatcaaagcccaacccgcatatcataa

SEQ ID NO: 51 - DNA, 1584 bp, Bacillus subtilis
atgatgcaaaaaattcagcgctttggaagcgcgatgtttgtgcctgttttattattcgcgttcgccggcattatcgtcggtatcagcacgctctttaaaaataaaaccctcatgggaccgctcgccgatcctgacggtttttggtatcagtgctggtatatcattgagcagggcggctggactgtttttaaccaaatgccgctcttattcgccattggcatcccggttgctttggcgaagaaagctcaggcacgcgcctgtttggaagcgcttactgtctacctgacattcaactattttgtcagcgcgatattgacggtatggggaggagcatttggcgtcgacatgaatcaagaggtcggcggaacgagcgggttaacgatgattgcgggcataaaaacgctcgataccaacatcatcggagccatctttatttcttcgattgtcgtctttttgcataatcgctattttgataaaaaactgcccgattttctcggcatctttcaaggctcaacatatatcgtgatgatttccttctttattatgatcccaattgcgttggctgtgtcttatatttggccgatggttcaatcgggaatcggctcgcttcaaagcttcctggttgcttctggggcggtgggcgtttggatatacacgtttttggaacggattttaattccgaccggccttcatcactttatttacacgccgtttatttatggcccggctgtagcggaaggcgggatcgtcacgtattgggcacagcatctcggcgaatattcgcaaagcgccaaaccgctgaaggagctctttccgcaaggcggattcgcgcttcacggcaactcgaaaatcttcggtattccgggtatcgccctggctttttatgtgacagccaaaaaggaaaagaaaaaactcgtcgcagggctgctgattcctgtcacactgacagcgattgtcgccggtattacagagccgattgagtttacgttcttattcatttcacctttcttatttgcggttcacgccgtgcttgccgccacaatgtcgacagttatgtatatggccggcgtcgtcggaaatatgggaggcggactgattgaggcggtaaccttgaactggattccgctctttggcagccacggtatgacatatgtgtatcaaattttgatcgggctctcgtttacagcaatttattttttcgtcttcagatttttaatcctcaaattcaatatcgctacaccaggacgggaaaaggatgaacagcaggaaacaaagctatattcgaaaaaggaatacagagaacgaaaaaacaaggatgaaacggcctccgctgctgaaacggctgatgacaccgcttttctgtatattgaagcgctgggcggaaaagacaacatcactgaagtcacaaactgcgccacccgcctcagagtcagtgtcaaggatgaaacaaaggttgaacccgacagcgtattccgcgcgcttggcgcacacggcgttgtcaggaacgggaaggcgtttcaggtaattatcggattaagcgtgccgcagatgcgggagcgtgtggaaaaaatattgaatcaataa

SEQ ID NO: 52 - DNA, 1365 bp, Bacillus subtilis
gtggaactgatcatcattctattggcgttaggtttgctgatgtttacggcgtatcggggattttctgtcatattgtttgcgccgatttgcgcgttattcgcggtgctgctgacagatccaagccatgtgcttccttttttttcatcaatttttatggagaagatggcgggttttattaagctgtatttcccagtgtttttgctcggtgctatttttggaaaggtcgttgaaatggccgggcttgcggcatcaatcgcgaaaacaattgtccggcttgtcggggcaaaaagagcgatacttgccattgtgctgatgggtgctgtcttgacgtacagcggtgtcagcctgtttgttgtcgtatttgctgtatatccttttgcgaaaaacatgttccaagaagcaaacataccaaaacgcctcatcccgggtacgattgctttaggagcttttacgtttacgatggacgcacttccgggaacgccgcaaatccaaaatgtcatcccgacgtcgtttttcaaaacagacatttatgccgccccttggctgggtttgatgggcgcagtgattgtgctggcagctgggatgctctatttggaatcaaggcggaagaaagcgcaggcatctggcgaaggctatggcggttttgattcgcagaatgctcctgctcctgaatcgattgagtccgcggctgaaccggacaaaagcccgattcggcacgcccttgcctttgtcccgcttatcctcgtcggtgcagtgaataaatatttcaccatttacctgccaaagtggtatccgaatggatttaattttccttccataggattaaaggagttcggcaggcttgatatttcttcagcggctgctatttggtcggtggagattgctttagtgattggcatcatcacaacgatattatttgattggagaagtgtgtttgcccaattgaaggaagggctgaatgaaggaattggcggcgccttgctggcatctatgaatacgggtgctgagtacgggttcggcggcattatcgccgcgctgccggggtttcataagctgagcagcggaatttcacatacttttaccgatccgcttgtaaatggcgccgttacgacaactgcgctggcgggaatcaccggctcggcttcgggaggaatgggcattgcgttaagcgcgatgtcagaacaatacttacaggcgattcaggcttacaatattccgccagaggtgatgcatcgggtcatttcaatggcatcaggcgggatggatacactgccgcataatggcgccgttatcacgctttctggccgtgacgggtttgacccaccggcaatcctatcgcgatatttttgcgatcacgctcattaa

SEQ ID NO: 53 - DNA, 717 bp, Bacillus subtilis
atgggaaaagtgctgtcatcaagcaaggaagctgcgaaactgattcatgatggggatacgctgatcgcgggagggtttgggctgtgcggcatccctgaacagctcattttgtctataagagatcagggagtaaaggatttaaccgttgtcagcaataactgcggagtcgatgactgggggcttggtttgcttctggctaacaagcaaatcaagaaaatgatcgcttcctatgtcggtgaaaataaaatttttgagcggcagtttttaagcggagagcttgaggtagagcttgttccccaaggaacgctcgctgagagaattcgtgcaggcggtgcaggcataccgggattttatacggcgacaggcgtcggcacctccatagccgagggaaaagaacataaaacattcggcggccggacttatgtgctggagcgaggcattaccggcgatgtggcgatcgtcaaagcgtggaaagcggacaccatgggcaatttgatttttaggaaaacggcgagaaatttcaatcccattgccgccatggcaggcaagatcacgattgccgaggcggaagaaatcgtggaagcaggagagctcgatccagatcacatccatacgccgggaatttacgtacagcatgtcgtgcttggcgcgagccaagaaaaacggattgaaaaacgaacagttcagcaagcatcgggaaagggtgaggccaagtga

SEQ ID NO: 54 - DNA, 651 bp, Bacillus subtilis
gtgaaggaagcgagaaaacgaatggtcaaacgggctgtacaagaaatcaaggacggcatgaatgtgaatctcgggattggaatgccgacgcttgtcgcaaatgagatacccgatggcgttcacgtcatgcttcagtcggaaaacggcttgctcggaattggcccctatcctctggaaggaacggaagacgcggatttgatcaatgcgggaaaggaaacgatcactgaagtgacaggcgcctcttattttgacagcgctgagtcattcgcgatgataagaggcgggcatatcgatttagctattctcggcggaatggaggtttcggagcagggggatttggccaattggatgatcccgggcaaaatggtaaaagggatgggcggcgccatggatctcgtcaacggggcgaaacgaatcgttgtcatcatggagcacgtcaataagcatggtgaatcaaaggtgaaaaaaacatgctcccttccgctgacaggccagaaagtcgtacacaggctgattacggatttggctgtatttgattttgtgaacggccgcatgacactgacggagcttcaggatggtgtcacaattgaagaggtttatgaaaaaacagaagctgatttcgctgtaagccagtctgtactcaattcttaa

SEQ ID NO: 55 - DNA, 774 bp, Bacillus subtilis
atgagaaaacaagtcgctttggtgacaggggctgccggcggaatcagattcgaaatcgcaagagaattcgcccgggaaggtgccagcgtcatcgtttcagacctccgtccggaagcatgtgaaaaagcagcctccaagcttgcagaagaaggctttgacgcggcggccattccgtatgatgtgacaaaggaagcgcaagttgctgatacggtgaacgtcatccaaaaacaatacggccgcttggatattctggtgaacaatgccggtattcagcacgtcgctccgattgaagagtttccgacagacacctttgaacagctgatcaaggtcatgctgacggctccctttattgcaatgaagcatgtttttccgatcatgaaaaaacagcagtttggcagaatcattaatattgcgtctgttaatggattagtgggctttgcagggaaatccgcttataatagcgccaagcacggcgtcattggactcacaaaagtaggggcgctggaaggcgcgccccacggcataacagtcaatgcgctctgtccgggttatgtcgatacccagcttgtacgcaatcagcttagcgatctatcgaaaactagaaatgtcccttacgactctgtacttgaacaagtcatttttccgcttgtgccgcaaaagcgactgctttccgtcaaggaaattgcggattatgccgtgtttttggcaagcgagaaggcgaagggcgtcactgggcaggctgtcgtccttgatgggggctacaccgcacaatga

SEQ ID NO: 56 - DNA, 1788 bp, Bacillus subtilis
atgtcacggctccttgtgaccttatgccaaaagcagaggaactggccgcagccatttctgccaacggaccgatcgctgtccgtcaggctaaatttgcaatcaataaaggattggagacagatcttgctacaggccttgcgattgaacaaaaagcgtatgaacaaaccatcccgacaaaagacaggagagaagggcttcaggcctttcaagaaaaaagacgggccgtatacaagggaatataaaagggaggcaatgctgatggattatgaaaaggaacgaacagaacgggctgaacggattcgaaaaggcggagcggaaaagtatcatcaaagcaatcgggaaaaaggcaagctctttgtcagagagcggctttcccttctctttgacgatgacattgagctagaagacgctttttttgcggaatgtatgtcagacgggcttcccgctgacggagttgtaaccgctatcggcaaaatcggcggccaaaccgtttgcgtcatggcgaatgattcaacagtgaaagcggggtcatggggagcaaaaacagttgaaaaaatcatcagaattcaagaaatcgccgaaaaattaaactgtccgctcatttatttagtcgattcggcaggcgcccgaattaccgaccaaatcaatgtctttccagggagacgcggtgcaggaagaattttttacaatcaagtcaaattatcgggacgcattccgcaaatctgtctgcttttcggaccatctgcggcaggaggcgcttatattccggccttctgtgatatcgtcgttatggtagacggtaacgcctccatgtatttaggttcgccaaggatggcggaaatggttattggagaaaaagtgtctctcgaagaaatgggtggcgcccgtatgcattgctcaatctccggctgcggagatattcttgcagaaactgaagaagaagccatacagctggtgcgggcttatttgtcttactttccggcaaattttcaagaaaaagcgcccattcatgagaaacgcccgccaaaacacttcgaaactccgcttgccgacgtcattccgcaaaatcaaaacgcaccttttgatatgcatgagctcattgagcgggtcatagatgaagactcattttttgagatcaaagccttatttgcaccggaattattgacgggcctcgcacgaatccacggacagcctgtcggcattgttgcaaaccagccgaaggtaaaaggaggcgtcttattccacgattcagcagacaaagcggctaagtttattaccttatgtgacgcttttcatatcccattgctgttcttagccgatatccccggttttatgattggcacaaaagtagaacaggctgggattatcagacacggagcgaaaatgatttctgcgatgtcggaggcaactgttccaaaactctctgtcattgtccgaaaagcttacggggcggggttgtatgcaatggcagggccggcatttgaaccggattgctgtctagcgctcccaaccgcccaaatcgccgtcatgggccctgaggccgctgtaaacgctgtctacgctaaaaaaatcgccgagctgccagaagaagagagagccgcatttatcagcagcaaacgggaggaatacaaagaggacatcaatatctaccgtctggcttcagaaatgatcattgatgctgttatcccagccaattcgctgcgtgatgagctggccaaacggctcaaggcatacatgacaaaggaaatgacatttaccaatcgaaagcatccggtttatccggtgtaa

SEQ ID NO: 57 - DNA, 783 bp, Bacillus subtilis
atgggagattctattctttttactgttaaaaatgaacatatggcgttgatcaccttaaacaggcctcaggcagcaaatgctctttcagcggaaatgcttagaaacctgcaaatgattatccaggaaattgaatttaactcaaacatccgttgcgtcatcctcacaggcaccggtgaaaaagcgttttgtgcaggggcagacctgaaggaacggataaaactgaaagaagatcaggttctggaaagtgtatctctcattcaaagaacggcggctttacttgatgccttgccgcagccggtcatagctgcgataaatggaagcgcattaggcggcggactagaattggcattggcatgcgaccttcgaatcgcaactgaagcagctgtgctgggacttccggaaacagggttagctattatcccgggcgctggagggacccaaaggctgccccggctgattggcagaggaaaagcaaaagaattcatttatacaggcagacgcgtgaccgcacacgaagcaaaagaaatcggccttgtagagcatgtcacggctccttgtgaccttatgccaaaagcagaggaactggccgcagccatttctgccaacggaccgatcgctgtccgtcaggctaaatttgcaatcaataaaggattggagacagatcttgctacaggccttgcgattgaacaaaaagcgtatgaacaaaccatcccgacaaaagacaggagagaagggcttcaggcctttcaagaaaaaagacgggccgtatacaagggaatataa

SEQ ID NO: 58 - DNA, 900 bp, Bacillus subtilis
atgccatatcctaaaaaagtgacaatcaaagaagtcggcccgcgtgatggcttacaaaacgagcccgtttggatcgcaacagaggataaaataacctggatcaaccagctttcccggacagggctgtcgtatattgaaatcacatccttcgttcacccgaaatggattccggcgcttcgagatgctatcgatgtagcaaaaggcatcgaccgagaaaaaggggtaacgtacgcggcacttgtcccgaatcaaagaggactggagaatgcacttgaaggaggcattaacgaggcttgcgtttttatgtccgccagcgagacgcacaacagaaaaaacatcaataaatccacttctgaatccctccatatactcaaacaagtaaacaacgacgcacaaaaagcaaacctcacaacaagagcctacctctcgactgttttcggctgtccgtacgaaaaagatgtccccattgaacaagtcattcgcctttcagaagctctatttgaatttgggatttctgaactgtcgcttggagatacgattggagcagctaatcccgcccaagtggaaactgtacttgaagctcttttggcacgattcccggctaatcaaattgccctgcattttcatgatacgagaggaaccgctctggccaacatggtcacagcactccaaatgggcatcacggtgttcgacggctcggcaggcgggcttgggggatgcccatatgcgccaggttcatcaggaaacgccgcaactgaggatatcgtgtacatgcttgaacagatggatatcaaaacaaatgtaaagctagaaaaactgctatctgcggccaaatggattgaagaaaaaatgggcaaaccgctgccgagcagaaatttacaggtgtttaaatcatcttga

SEQ ID NO: 59 - DNA, 1335 bp, Bacillus subtilis
atgtttacaaaagtactgatcgccaaccgcggtgaaattgcaatgagaattatccgaacatgcagccgtctcggcattaaaacggtcgctgtttattcagaagcagacaaggacgcgccccatacaaaagccgctacagaggcatatttgatcggggaatcgagagtcagtgaaagttatttaaatatagagagaatcataaagacggcgaaaaaagcaaaagccgacgcgatccacccgggatatggattgttatcagaaaacagccggttcgctgaacgctgcaagcaagaaaacatcgtgtttatcggaccttcccctgatatcatcgcaaagatgggcagcaaaattgaagcgcgaaaagcaatggaggctgcaggtgtccctgtggtgccgggcgtttctgaatccctcggagatatagaggcagcctgccgcaccgcaagtcaaatcggctatcctgtcatgctgaaagcttcagcgggcggaggcggcatcggaatgcagcgtgttgaaaatgaagaagcattaaaaaaagcgtacgagggaaacaaaaagcgcgcagcagattttttcggtgacgggtctatgtatatagaaaaagttattgaacatgcgcgccacatcgaggttcagcttttggccgatcaacacggccatacagtacatctgtttgaacgtgattgctctgttcagaggcgccaccaaaaagtcattgaagaagcaccgtctccatttgtagacgatgaactaagaatgaagatcggtcaaacagcggtaaaagcagcgaaggcaatcggctatacgaacgcaggcaccatcgaatttatagttgaccagaaacaaaatttttatttcctcgaaatgaatacgagactgcaagttgaacaccccgtgactgaagaaataacaggcctggacttagttgagcagcagctgcggattgctgcgggccatacactcacattctcccaaaaagacatccaacggaacgggcatgcgatagaggttcgaatatacgcggaagatcccaagaccttcttcccgtcaccaggtacgatcactgcgttttcacttcctgaccaaaaaggagtcagacacgaatgtgcggtagcaaaagacagcaccgttacccctttttatgacccgatgatcgctaagatgattgtcaaaggccaaaccagaacagaagcaattgaaaaactagagacagcgcttcgcgactatcgtgtagagggaatcaaaacaaaccttccgctcctcatacaggctgcggcaacaaaggcatttaaagaaggggatgtcacgactgactttttgaaacagcacctataa

SEQ ID NO: 60 - DNA, 1650 bp, Bacillus subtilis
atggctgaactcatccattccacaatcggcaggctgctggaacaaacagctgatgcgtatcccgatcgagatgctgttgtgtatccagaccgaaatatccgctatacgtacgctcaatttgacagtctgtgccgtcaaaccgctaaaggtctcatgcggatggggattggaaaaggagaccacgtcgccatatgggcttctaatatctctgaatggcttgccgtccagttcgcaactgcgaagatcggagccgtgctcgtgaccgtcaataccaattatcaagcacatgagcttgattacttgttaaagcaatcggatgccgcggcgcttattatcatggattcatacaggggcacttcttatccagacatcgtgaacagtttaattccagaactgcaagaagcaaagcccggccaactgaaatctgaacgctatccctttttaaaaacgctgatctatattggcaataaacgattgtctggcatgtatcattgggacgatacagagatattggcgaaaacagtgacagatgctgagcttgaagagagaatgaacagcctggataaagacaatgtgattaatatgcaatacacatcaggaacgacagggtttccaaaaggcgtgatgctgacccatttcaatgtcatcaataacgctgctaatatcgctgaatgtatggctttaacctctcaagaccgcatgtgcatccctgttccgttttttcactgctttggatgcgtccttggggttttggcatgtgtatccgtcggggcagccatgatacccgtgcaagaatttgatcccgttaccgtccttaaaacggtagaaaaagagaaatgcacagtgctccatggtgtgcctaccatgtttatcgccgagctgcatcatccggattttgatgcatatgatctatcgacgctccgaacaggaatcatggccggctctccctgtccaagtgaagtgatgaaagctgtgattgaaaggatgggcatgaaagacattacgatcgcctatggacaaaccgaagcctcgccagtcattacacaaacgagagcaaatgattccttcataagaagagtcgaaacaaccggccgtgccctgccacatactgaggtgaaaattgtagaacccgggacatgtcaagaagttcaaagaggcatgcagggagaactgtgcacccgtggctatcacgtcatgaaaggctattataaagacaaagatgcgaccagaaaagcaatcaatcatgacggatggctgtttaccggagatcttgctgtcatggatgaagacgggtactgccgcatcaccggaagattaaaagatatgctcatcagaggcggcgagaacatttatccgcgggaaattgaagaatttttataccagcatcccgctgttttagatgtacaggtggttggtgtgcctgacgccaaattcggggaggaagctgcagcctggattaaactgaaagacggtaaaagcgtttcacctgatgagcttaaagcctattgcaaagggaaaatcgcccgccacaaaattccgcgttatgttatttttacggatgactatccgatgacggcctcaggcaaaattcaaaaatataaactgcgagaaaaaacgattgaaatgttcaacttatcatcaagtcaatga

SEQ ID NO: 61 - DNA, 1014 bp, Bacillus subtilis
ttgaaaacgataacaattgcagctgaagaagcaaaggaactcgtttggcaaaagctggacggtgccggtttgaatgaacgagatgctgaaaaagtggcagatgttctcgtgcacgctgatttgcgcaatgtacattcgcatggcgtgctgcacacagaacactatgtgaacaggcttttagcgggagggatcaatcctggggcacagcctgtttttaaagagacggggcctgtgaccggggtgcttgacggagacgatggtttcggtcatgtgaattgcgacatggcgatggaccatgcaattgacatggcgaagaaaaaaggagtcggcatggtcacggccgtaaacagcagccattgcggagcgctaagctattttgtgcaaaaagcggctgacgaaaagctgatcggaatggcaatgacgcatacagacagtatcgttgtcccatttggggggaggactcctattttagggacaaatccgattgcttacggagttccggctaagcataaaaaaccgtttatcctagatatggcgacatccaaagtggcttttgggaagattctgcaggcccgtgaagagggcaaagaaattcctgaaggatggggagtcgatgaaaacggagaagcagtaactgatcctgacaaggtcgtctcactttcaacattcgggggcccgaaaggctatggactatcgattgtagtggatgtgttttccggattgctggcgggcgcggcttttggccctcatattgccaaaatgtacaacggccttgatcaaaaaagaaagctggggcattacgtttgcgcgatcaatccatccttttttactgactgggatacgtttttagagcagatggatgccatgattgatgaactgcagcaatcaccgccggctgttggattcgaaagagtgtatgtgcccggcgagatcgagcagctgcatgaagaaagaaataagaaaaacggaatttctatcgcccggagcgtgtatgaattcttaaaaagcaggtga

SEQ ID NO: 62 - DNA, 1020 bp, Bacillus subtilis
atgaaagcggttcaagtgcgaaaagcgtatgatctggtgacagcggaggtgaagaagccagttctttcaaaggatgatgaagtgctcgtgaaagtcaagcgagtcggcatttgcggttcagacatgcacatttatcatggaacgaatccgctcgctaccctcccgagagtcatcggacacgaggtaacgggacaagtggaggcagttggtgcgaatgtacagagcctaaaacccggtgatcatgtggtgattgagccgatttcttattgcggatcgtgctatgcctgccgcaaagggcggccgaatgtttgcgccaagctttctgtatttggcgtacatgaggacggaggcatgcgggaatatattgtgcttccggaaagacagcttcacgcggtctcaaaggacttgccttgggaggaagcagtcatggccgagccttatacgataggcgcccaggcagtgtggagaggccaggtggaaaaaggtgataccgtcctgatccagggagcggggcccatcgggatctgtgtgttaaaaatggcaaaactggcgggcgctgctgtcatgatgactgacttgaacaacgagcggctggcatttgcgaaagaaaacggcgccgatgctgttgtaaatgtccaagcagaacatgttgccgagcgggtccttgaatggactgggaatgaaggagcaaacgtggtcattgatgctgtttgcctgccggagacttttgcactttcaattgaggctgtgtcaccggcgggacatgtggttgtgcttggatttgatgaaagagcggctcagatttctcagctgccaattacaaaaaaagaagtcacgataaccggatcccgattgcagaccaatcagtttccaaaagtggtagagcttttgaatggaggccggttaatgcataacgggctggtgacccatacattttcagttgatgacgttcatcatgcatttcagtttattaaggagcatccagatcaggtgcggaaagccgtcatcacgtttgattaa

SEQ ID NO: 63 - DNA, 1080 bp, Bacillus subtilis
atgaatatgacattccgatggtatggacgaggcaacgatacagtcacacttgaatacgtgaagcaaattcccggtgtcaaaggcatcgtttgggctctccatcaaaagcccgtcggcgacgtgtgggaaaaagaagaaatcagagccgaaactgaatatattcaatcctatggttttcatgctgaagttgtagaaagcgtgaatgttcacgaagcgattaaacttgggaacgaagaacgcggccggtatattgaaaactacaagcaaacgatccgcaaccttgccggatttggcgtgaaagtgatctgctataattttatgccggtttttgattggacacgcacggacatgttccggccgctagaagatggatcgaccgctctgttttttgaaaaggccaaggtggaaagccttgatcctcaagagctgattcggacggtggaggaagcatccgacatgacactgccggggtgggagcccgaaaaattggctcggatcaaagagctttttgctgcctacagaacggtcgatgaagaaaagctatgggacaatttatcattctttttgcaggaaattcttcctgttgctgaggcctatggtgttcaaatggccattcatccggatgacccgccgtggccgattttcggactgccgcgcattatcacaggagaggcaagctataagaaactgcgggcgatatcagattcaccgtctaattgtatcaccctttgtacaggttcaatgggagccaatcccgctaacgacatggtggagatcgctaaaacgtatgccggcatcgctccattttcacatattcgcaatgtgaaaatttatgagaatggcgattttattgaaacatctcatttaacaaaggatggttcgatcaacattcaaggcgtgatggaagaactgcataagcaggattacgaaggatatgtcagaccggatcatgggcgccatctttggggcgagcaatgccgcccgggatatggcttatacgatcgggcacttggcatcatgtatttgaacgggctgtgggacgcttatgaagcaatggcaaaaaaagaggtgggcatatga

SEQ ID NO: 64 - DNA, 837 bp, Bacillus subtilis
atgatcccgctgcatgagaacctggctggtaaaacggctgtcatcactggcggcagcggcgtgctttgctctgcgatggcccgggagctagcccgtcatggcatgaaggtggcgattttgaatcggacggctgaaaaaggccaagcggtcgtgaaggagataacggcggctggcggcacagcgtgcgctgttgctgcggatgtgctggacaggatgtcactggagcgggcaaaggaagacatccttggccaatttggcgctgttgatctgttaattaacggggctggcggcaatcatcctgacgcgataaccgatgtggagacatatgaagaagcgggagaaggccaatccttttttgatatggatgagaggggctttctaactgtattctccaccaacttcaccggtgcgtttctggcctcgcaagtgtttggtaaagaactgctgaaggcggattcacccgcgatcatcaacctttcttccatgagtgcttattcacctatgacgaaggttccggcatacagtgctgcgaaagcatccatcaataattttacgatgtggatggctgttcattttgccgaaaccgggctgcgggtcaatgcgattgccccaggcttctttctgacaaaacaaaatcatgatctgctgatcaaccaagacggaacgttcaccagccgatctcacaaaattattgcgggaacaccgatgaagcgcttcggaaaaccggaggatttgctcggtacgctcctttggctggcggatgaatcctattccggttttgtcactgggatcaccgttcctgtcgatggaggatttatggcttattcaggtgtgtaa

SEQ ID NO: 65 - DNA, 1269 bp, Bacillus subtilis
atgttttcaaaagataagcttcccgttatcctttttttgttcctggcaggggtgattaattacctggatcgctcggcgctttccattgcagctccttttattcaggatgatctcacattgtctgccacacaaatgggcttgattttcagcagtttttcgataggttatgccatttttaattttcttgggggcgtggcatccgaccgctatggggcaaagctgaccttgtttgtcgcgatggttgtttggtcgctgtttagcggagcagtcgccctcgcttttggctttgtcagcctgctgattatacgcattctcttcggaatgggagaaggcccgctttcggcgaccatcaacaagatggtgaacaactggttcccgccgacccagcgggcgtccgttatcggtgtaaccaacagcggcacgcccctcgggggagccatttccggcccgatagtcggcatgatcgcagtggcgttcagctggaaggtatccttcgttctcattatgattattggattgatatgggcagtgctctggttcaagtttgtcaaagaaaagccgcaagagacgatcaaggaagcaccggcaataaaagcagaaacgtctcccggagaaaaaattccgctcaccttttacctgaagcaaaaaacagtcctgttcacggcgttcgcttttttcgcttacaactacatcctcttcttctttttgacatggtttccgagctatcttgtcgacgagcggggattaagtgttgaatcgatgagtgtcatcacggtcataccgtggattttaggatttatcgggctggctgcggggggatttgtttctgactatgtgtacaaaaaaacggcccgaaaaggtgtgctgttctcgcgcaaggttgtgcttgtcacgtgtttgttttcatcagctgtcctgattggttttgccgggcttgtggcaacgactgcgggggctgtcactcttgtcgctctgtcagtgttctttctttatttgaccggtgctatctattgggctgtcattcaagatgtggttgatcaaaacaatgtcggttctgttggcggcttcatgcatttcctcgccaacacggcaggaattatcggcccggctttaaccggatttattgttgaccaaacaggcacgttttctggagcatttttgcttgccggtgggctggctgtcttcgcttcacttgctgtgattcgttttgtccgtccaatcattggtaagccagcgggaacagaagctgagaatcctgtgtcttattaa

SEQ ID NO: 66 - DNA, 705 bp, Bacillus subtilis
gtgcgcatcgggggttttgggacaggacgtatcgccgcgggcattgatttcagcttgatccgcaaacaccctaaaatcttttggggatacagcgatattacgtttttacatactgccattcatcaaaacacaggtcttgtcactttccatggcccgatgctcagcacggatattggccttgacgacgttcacccgctgacaaaagcgtcatataagcagctcttccaggagacggaattcacctatacagaagagctttctccgctgaccgagcttgttcctggaaaagcggaaggcgagcttgtcgggggaaatctgtctttgctgacgtctacactgggcacgccatttgaaattgatacgagaggaaagcttctgtttattgaagatattgacgaggagccttatcaaatcgaccggatgctgaatcagctgaaaatgggggggaagctgacggacgcggcgggaattctagtttgtgattttcacaattgtgtcccggtgaagcgagagaagtctctctcgcttgagcaggtgctggaagactatattatttctgcgggcaggcctgctctgagaggatttaaaatcggccactgctcgccaagtattgccgttccgatcggtgcgaaagctgctatgaatacagcagaaaaaacagccgtaatagaggcgggcgtttcagaaggggcgctgaagacatga

SEQ ID NO: 67 - DNA, 1101 bp, Bacillus subtilis
atgaaaatcattcgaatcgaaacaagccgaatcgctgtcccgctgacaaagccgtttaaaaccgcacttcgcactgtgtatacggctgaatcagtcatagtaaggattacttatgacagcggtgcagtcggatggggagaagcacccccgacgttagtgattacaggagacagcatggatagcattgaaagtgccatccaccatgtgttgaagccggcattgcttggaaaaagcctggcgggctatgaggccattctgcacgacatccagcatcttcttacaggaaatatgagcgcgaaggctgctgtagaaatggctctatacgacggctgggcgcagatgtgcgggctgccgctttatcaaatgcttggcggatatcgagatacgctggaaacagattatactgtcagtgtcaactcacctgaagagatggcagctgatgccgagaattatctcaaacaaggctttcaaacgctgaaaataaaggtcggaaaagatgatattgcaacagatatcgcccgtatccaggaaatcagaaaacgtgtcggatcagctgtgaaactgcgtttagacgctaatcaggggtggaggccgaaggaagcggtaactgccattcggaaaatggaggatgcgggcctaggcattgagcttgtcgagcagcctgtccataaagatgatctcgctgggcttaaaaaggtgacagatgcaacagatacgccgattatggctgatgaaagtgtttttacaccgcgccaggcgttcgaagttctgcaaacccggagcgcagacttgatcaatattaaattgatgaaagcgggcggcatcagcggagcagagaaaattaatgccatggcggaggcctgcggggtggagtgtatggtcggcagcatgatcgaaacgaagctgggcattacggccgcggcgcattttgcggcaagcaagagaaacatcacacgctttgattttgacgcgccgctgatgctgaaaacagatgtattcaatggcggcataacatatagcggcagcacgatttcgatgcctggcaaaccgggcctcggaatcatcggggctgcgcttttgaaaggggaaaaagagcaatga

SEQ ID NO: 68 - DNA, 891 bp, Bacillus subtilis
atgatgcacactgtcatatcagcagtggccaacatctggacagcgcctgattcacctcgtccgtctgatcaattcatgcttcaaccgactgtaatgatcagagactggctggagcgcatgacgtatgatgaacggcttggattatgtacagacaatgtaatccaaactcaggttctctttggcgaaaaggtacttgtgacggcggaacagggggaatgggtttctgtgatcgtgcctagccagccatcccgaaaggatccgcgcggatacccgggctggatgaaaaagtaccagctggaaaaaacaaagcccatccatacacaacacgatgtgatgatcagcaaacctgctgcctttttgtacagaagcaatggggaaaaggagatcgaattaagctttttgacagttctgccccttattgcaaaagaaaacggatattttaaggtttcgaccgtttttggggaaaggtttgtgaggcaaagtgatgcagtgcctgtcagccaacagaaagggactgctgaagacatcattcaaacgggtgcgttttttcttggccttccctacctgtggggagggatcagcgggtttgggtttgattgctccggatttatgtacagtatatttaaggcgaatggatacagcatcccccgtgatgcgggagatcaggctaaggcagggaaggttgtcccgcttgatgatatgaaagccggtgatctgctgttttttgcttatgaggaaggaaaaggagcgattcatcacgtcggtctgtatgtaggcggcgggaaaatgcttcattctccaaagacagggaagtcaatcgaaatcctcacattaacagagacaatctatgaaaaagaattatgtgcggtgcgccgctgtttttcagaataa

SEQ ID NO: 69 - DNA, 984 bp, Bacillus subtilis
atgaggcgactgcctttgttagaggtcagccagctgaaaatgcattttgacgcagggaaaaagcggacagtcaaagctgtcgacggggtcacctttcagattcgtgaaggagaaacgttcgggctagtcggggaatcagggtgcgggaaatcaaccttggggagagtgctgatgcgcctttatcagccgacagaaggaagcgtgacataccgcggcacaaatcttcatgcactaagtgaaaaagagcagtttgccttcaaccgcaaactgcagatgatttttcaggacccttatgcttcacttaacccgcgcatgaccgttcgagaaattattttggagccgatggagattcataatctctacaatacccataaagcacggctttccgtcgtggacgagctgcttgaggcagttgggcttcaccccgattttggcagccgttatccgcatgaattcagcggcgggcaaaggcagagaatcgggattgcgagagcactgtcgctgaatcctgaatttatcgtggcggacgaaccgatttctgcacttgatgtctctgttcaagcgcaggtggtcaacctgctgaagcggcttcaaaaagagaaagggcttacgtttttattcattgcccatgatctttcgatggtgaagcatatcagtgacaggatcggtgttatgtacttaggacacatgatggaaattacagagagcggcaccttgtatcgtgaaccgctccatccctatacaaaggcgcttttgtcctcgattccgattccagatcctgaattggaggacaagcgtgagcgtattctcttgaaaggggagctgccgagcccggtcaatccgccaagcggctgcgtgtttcgtacccgctgtccggagcgatgcctgaatgtggagaatctcgtccccagcttcaagaaatcgaacccggccgttttgtcgcttgccatttgtatcgaaatgctgaaacgaaggaaaaagtaa

SEQ ID NO: 70 - DNA, 1416 bp, Bacillus subtilis
ttgaaaaacaaatggtataaaccgaaacggcattggaaggagatcgagttatggaaggacgttccggaagagaaatggaacgattggctttggcagctgacacacactgtaagaacgttagatgatttaaagaaagtcattaatctgaccgaggatgaagaggaaggcgtcagaatttctaccaaaacgatccccttaaatattacaccttactatgcttctttaatggaccccgacaatccgagatgcccggtacgcatgcagtctgtgccgctttctgaagaaatgcacaaaacaaaatacgatctggaagacccgcttcatgaggatgaagattcaccggtacccggtctgacacaccgctatcccgaccgtgtgctgtttcttgtcacgaatcaatgttccatgtactgccgctactgcacaagaaggcgcttttccggacaaatcggaatgggcgtccccaaaaaacagcttgatgctgcaattgcttatatccgggaaacacccgaaatccgcgattgtttaatttcaggcggtgatgggctgctcatcaacgaccaaattttagaatatattttaaaagagctgcgcagcattccgcatctggaagtcatcagaatcggaacaagagctcccgtcgtctttccgcagcgcattaccgatcatctgtgcgagatattgaaaaaatatcatccggtctggctgaacacccattttaacacaagcatcgaaatgacagaagaatccgttgaggcatgtgaaaagctggtgaacgcgggagtgccggtcggaaatcaggctgtcgtattagcaggtattaatgattcggttccaattatgaaaaagctcatgcatgacttggtaaaaatcagagtccgtccttattatatttaccaatgtgatctgtcagaaggaatagggcatttcagagctcctgtttccaaaggtttggagatcattgaagggctgagaggtcatacctcaggctatgcggttcctacctttgtcgttgacgcaccaggcggaggaggtaaaatcgccctgcagccaaactatgtcctgtcacaaagtcctgacaaagtgatcttaagaaattttgaaggtgtgattacgtcatatccggaaccagagaattatatccccaatcaggcagacgcctattttgagtccgttttccctgaaaccgctgacaaaaaggagccgatcgggctgagtgccatttttgctgacaaagaagtttcgtttacacctgaaaatgtagacagaatcaaaaggagagaggcatacatcgcaaatccggagcatgaaacattaaaagatcggcgtgagaaaagagatcagctcaaagaaaagaaatttttggcgcagcagaaaaaacagaaagagactgaatgcggaggggattcttcatga

SEQ ID NO: 71 - DNA, 828 bp, Bacillus subtilis
atgctcaagtcaataaagagtagcggtgtcacagcagttttggaccatgacggctttaataaacgaatcagagtggttcgttatgacggagccattgagaaggccctgccggatatcgtggcagcggcaaaagaagagaatgcagaaaaaatcattgtctatgcgaagcagcatgatgagccgatccttgccaaacaattatttgcgccggagggctatctaaagggctattatctcggccattcggcttgtgtcatggtacgttacctttcagaaagccggagacaaacagattcttatacagaggaacaggagatcatcgaagccatatatcgcacagcgccccgtcttcgcaacgacagtacacccgtttttacgatgagaaaagcagaaacaaacgacatgtaccagctatcgatgctgtataaaaaagtattccgcacgtacccaaccccggtatttgaccccgcttatattgaaaagacgatgaatgcaaatacggtgtattatatcatgcttgatcatgaccgcctgatcagcgcagcaagcgcagaaatcaatccagagcttgggcatgcagaaataaccgattgcgctgtgctgccggaatatcgcggccattcgttaacaagctttttaatcgaggcgttagaaaaagaaatggctggagaggatatcgttcatgtgttttctctcgcccgtgcttcgtcttttgggatgaatgctgtgttgtaccattcaggttatcagtatggcggaaggctgatcaataattgctttatagccgaaggccttgaaaacatgaatatttggtgcaagcaactgtaa

SEQ ID NO: 72 - DNA, 654 bp, Bacillus subtilis
atgggcttgggagtagcagaaagagaacagattgcaaaacgcgctgctactgaaattaagcagggcatgattgtgaatctcggtatcggtatcccttccttggtaccgaactttttgaagcctgacatgcaggtcatgtttcaagcggaaaacggtgtccttggcattggagaaagtcccgaaaagggagaagaggatgcgcatttatgcaacgccgcgggatatcctgtccgcgctgtaaaaggggcttcttattttgatacaaccatgtcttttgcgatgatcagaaaaggcaaaattgacattacgattttaggcgccctgcaggtgagccaatcaggagatttggcaaattggcttgttccgggaaaaaaggtgcctggtatgggcggggcgatggagcttgcccaaaaagcgaaaaaagtggttgtcgtcatgagtcatacagatcaaaagggaaggcctaaattaacagaaagatgtacgctgccattaactgctgcaggctgtgtagatttgattattaccgaaaaagcggttcttgaggtcgatagccatcacttcattttaaaagagctgatgaatggctcgacaatcgatgaggtgacgaggctgacagaagctgaaatcaaaatagatatgcctttttcttaa

SEQ ID NO: 73 - DNA, 690 bp, Bacillus subtilis
atggcgccatttcaaaaagcaatcagcattgacacagcaattgcagatgttcgggatggatcggttctgatgtttggcggttttgggggagtcgggtcgcctccttcattgattgaagcgatattggacagcggtgtaacggatttgactgtgatttgcaatgacgccggtttcccggatatcggaatcggcccgcttattgttaatcaacgggtcaaaaccctgatcgcctcgcatatcggttccaatccagtagccggaaaacagatgacagaggggacgttagaggttcaattttcacctcagggaacgcttgcggaacggattcgcgccggcggagcggggcttggcggtattttaaccgacgtgggcattgataatcaaatggtttgcgaaaaaaaggacatcgtaacagtggcgggaaaacgatacttgattgaagaggcgctgactgctgattttgctttcatcaatgcttacattgcagatgaattcggcaatctaacgtatgacaaaaccgcgcgcaatatgaacccgcttatggcaatggccgccaggagaacctttgccgaagctgagcgtatcgttccgatgggggagatttctgaagaaatgattgtcacaccgggggtttttgttgagggggttgtacgaagcgagggagtgaagtggaaatgggcttgggagtag

SEQ ID NO: 74 - DNA, 1335 bp, Bacillus subtilis
atgagcagttatttgattaagccagagcttagctcggcctatccggttgtcagttatgcgaagggttcatatgtttatgatcagaccggaaaaaaatatctcgacggctcgtcaggtgcggtgacatgtaatatcggccacggagttcgtgatgtgactgagaagctgaaagaacagcttgatcaggtgtcttttgcttaccgctcacagtttacgagtgagcccgccgagcaattagccgctctcttggcacaggagctgcccggagatgtgaattggtctttttttgtcaacagcggatcagaagcgatagaaacagctatgaaaatcgccattcagtattggcaggaaaaaaagcaaacacaaaaatccatctttttgtctcgatggagcagttaccacggaataactttgggagcgctttcattgtctggtttttatgaaaggagataccggttcacccatctcattgagcggtatccagctatctcagctccacatatttatcggctgaatcacgagacggaagaagactttgttcagactgcagctgatgaactggacaccatgattaaaagaatcggaagccaattcatcgccggctttgtggctgagcctattattggtgctgcaggagcagcgattactccgcctccgggatattatgagagattaagtgaggtatgccgcacacacgatgtgctttttattgcagatgaagtgatgacggggcttgggagaacaggaaggatgctcgcgacagagcattgggataccgtacctgatattgctgtactggggaagggactcggtgcggggtatgcacctattgctgctgccgtcgtatctgattctattattgaaaccataaaacaagggtcaggtgtgattatgagcggtcacacatatagtgcacatccctattcagccaaagctgctcttgaagttttgcgatatgtgttaaagcacggcttgatcaaacaatcagaaaaaaagggcgctgtgctgaagaagaagcttgatgaggcggcatctcaaagcggcattataggtgaggtgcgcggaaaaggactgctattaggcattgaatttgtggcagaccaaaaaacgaagaaagtgtttccgccagagcaggcgataacccagcttattgtcagcgaggcgaaaaaacgcgggctgattgtttatccttccaaagctggaatagacagtggagaaggagatgctgtcattattgctcctccttttactatttcagacggtgaaatggaagagcttatctctattttttcagaaacagttgcagcggtcgaaaaaaacttaaaaaaggattga

SEQ ID NO: 75 - DNA, 912 bp, Bacillus subtilis
gtgatcacaagagattttttcttatttttatccaaaagcggctttctcaataaaatggcgaggaactggggaagtcgggtagcagcgggtaaaattatcggcgggaatgactttaacagttcaatcccgaccattcgacagcttaacagccaaggcttgtcagttactgtcgatcatttaggcgagtttgtgaacagcgccgaggtcgcacgggagcgtacggaagagtgcattcaaaccattgcgaccatcgcggatcaggagctgaactcacacgtttctttaaaaatgacgtctttaggtttggatatagatatggatttggtgtatgaaaatatgacaaaaatccttcagacggccgagaaacataaaatcatggtcaccattgacatggaggacgaagtcagatgccagaaaacgcttgatattttcaaagatttcagaaagaaatacgagcatgtgagcacagtgctgcaagcctatctgtaccggacggaaaaagacattgacgatttggattctttaaacccgttccttcgccttgtaaaaggagcttataaagaatcagaaaaagtagctttcccggagaaaagcgatgtcgatgaaaattacaaaaaaatcatccgaaagcagctcttaaacggtcactatacagcgattgccacacatgacgacaaaatgatcgactttacaaagcagcttgccaaggaacatggcattgccaatgacaagtttgaatttcagatgctgtacggcatgcggtcgcaaacccagctcagcctcgtaaaagaaggttataacatgagagtctacctgccatacggcgaggattggtacggctactttatgagacgccttgcagaacgtccgtcaaacattgcatttgctttcaaaggaatgacaaagaagtaa

SEQ ID NO: 76 - DNA, 1548 bp, Bacillus subtilis
atgacaacaccttacaaacacgagccattcacaaatttccaagatcaaaactacgtggaagcgtttaaaaaagcgcttgcgacagtaagcgaatatttaggaaaagactatccgcttgtcattaacggcgagagagtggaaacggaagcgaaaatcgtttcaatcaacccagctgataaagaagaagtcgtcggccgagtgtcaaaagcgtctcaagagcacgctgagcaagcgattcaagcggctgcaaaagcatttgaagagtggagatacacgtctcctgaagagagagcggctgtcctgttccgcgctgctgccaaagtccgcagaagaaaacatgaattctcagctttgcttgtgaaagaagcaggaaagccttggaacgaggcggatgccgatacggctgaagcgattgacttcatggagtattatgcacgccaaatgatcgaactggcaaaaggcaaaccggtcaacagccgtgaaggcgagaaaaaccaatatgtatacacgccgactggagtgacagtcgttatcccgccttggaacttcttgtttgcgatcatggcaggcacaacagtggcgccgatcgttactggaaacacagtggttctgaaacctgcgagtgctacacctgttattgcagcaaaatttgttgaggtgcttgaagagtccggattgccaaaaggcgtagtcaactttgttccgggaagcggatcggaagtaggcgactatcttgttgaccatccgaaaacaagccttatcacatttacgggatcaagagaagttggtacgagaattttcgaacgcgcggcgaaggttcagccgggccagcagcatttaaagcgtgtcatcgctgaaatgggcggtaaagatacggttgttgttgatgaggatgcggacattgaattagcggctcaatcgatctttacttcagcattcggctttgcgggacaaaaatgctctgcaggttcacgtgcagtagttcatgaaaaagtgtatgatcaagtattagagcgtgtcattgaaattacggaatcaaaagtaacagctaaacctgacagtgcagatgtttatatgggacctgtcattgaccaaggttcttatgataaaattatgagctatattgagatcggaaaacaggaagggcgtttagtaagcggcggtactggtgatgattcgaaaggatacttcatcaaaccgacgatcttcgctgaccttgatccgaaagcaagactcatgcaggaagaaattttcggacctgtcgttgcattttgtaaagtgtcagactttgatgaagctttagaagtggcaaacaatactgaatatggtttgacaggcgcggttatcacaaacaaccgcaagcacatcgagcgtgcgaaacaggaattccatgtcggaaacctatacttcaaccgcaactgtacaggtgctatcgtcggctaccatccgtttggcggcttcaaaatgtcgggaacggattcaaaagcaggcgggccggattacttggctctgcatatgcaagcaaaaacaatcagtgaaatgttctaa

SEQ ID NO: 77 - DNA, 1398 bp, Bacillus subtilis
atggagtcttttttcaatagtttgattaatattccaagtgatttcatctggaaatacctattttatattttaatagggcttggattattttttaccatacgttttggttttatccaattccgttattttattgaaatgttcagaatagtaggggagaagccggaaggaaataaaggtgtttcatctatgcaggcattctttatttcggccgcatcccgagtcggcacagggaatttgactggtgtagccttagcaattgcgacaggcggaccaggcgctgtattttggatgtgggtagtggctgcagtaggcatggcttcaagctttgtcgaaagtacattagcacagctttataaggtaagagacggggaggatttccgcggagggccggcctactatattcaaaagggtcttggtgccagatggcttggcatcgtttttgcaatcttaattaccgtctcattcggcttgatttttaacgctgttcaaacaaatacaattgctggagcattggatggcgcattccatgtaaataaaatagttgtagccatagttctggcggttttaactgcgtttatcattttcggcggtttaaaacgtgttgtcgctgtttcacagctaattgtgccggttatggcaggcatttatattcttatcgctttatttgttgtcatcacgaatattacggctttccctggcgttatcgctacaattgttaaaaatgctttaggttttgaacaagtcgtcggcggcggaataggcggcatcatcgttatcggtgcgcaacgcggacttttttcaaacgaagcaggaatggggagcgcaccaaacgcggctgcgacggctcatgtatcccatccggcaaagcaaggctttattcaaacattaggcgtatttttcgatacatttatcatatgtacgtccacagcatttattattttgctgtacagtgtaacgccaaaaggcgacggcatccaagtcacacaggctgctcttaaccatcacattggaggctgggcgccgactttcatcgcagtcgcaatgttcttgtttgcattcagttcagttgtcggcaactattattatggcgagacaaacattgaatttattaaaacaagcaaaacatggctgaacatttaccgtatcgctgttattgctatggttgtgtatggatctttatcaggcttccaaatcgtttgggatatggcggacctctttatgggtatcatggcgctgatcaacttaattgtgattgcgctgctgtcaaacgttgcttacaaagtgtataaagattacgcgaaacagcgtaagcaaggacttgatcctgtgtttaaagcgaaaaacatcccagggctgaaaaacgctgaaacatgggaagatgagaaacaagaagcataa

SEQ ID NO: 78 - DNA, 675 bp, Bacillus subtilis
atgaacacgattgattgggaattcatgatatcagcgttcccgactttaattcaggcccttccgatcaccttgtttatggcaatagcagctatgatttttgccattatcggaggacttattctcgcactcattacaaaaaacaaaattccagtgcttcatcagctgtcaaagctgtatatatcctttttccgaggcgtgccgacacttgtacagctgttcttaatctattacgggctgccgcagctatttccagagatgagcaaaatgacagctctcacagctgccatcatcgggttaagcttaaaaaacgcagcttatttggcagaaatcttccgggccgccctcaattctgttgatgacgggcagctggaggcgtgcctgtctgtcggtatgacaaaatttcaggcatacagacggattattttgccgcaagcgatccgaaatgcgattccggcaacgggcaatacatttatcgggctcctgaaagaaacgtcactggcctttacattaggggtcatggagatgttcgcccaagggaagatgtacgcttcaggaaacctcaaatattttgagacgtatttggcggttgcgatcgtctattgggttcttaccattatctacagcattttgcaggacttgtttgaacgtgccatgagcaagccataccggacttag

SEQ ID NO: 79 - DNA, 795 bp, Bacillus subtilis
atgaagatgaaaaaatggacagtgctggtcgttgctgcattattagcggtgctctcagcttgcggcaatggaaacagcagcagtaaagaggatgacaatgtgcttcatgtcggtgcgacaggacaaagttacccatttgcttataaagaaaacggaaagctgacaggctttgacgtggaagtgatggaagcagtcgctaagaaaattgacatgaaactggactggaagctgcttgaattcagcgggctgatgggagagcttcaaacaggcaagcttgacaccatttccaaccaggtagctgtgacagacgaacgtaaggaaacgtataactttacgaaaccatacgcttatgcgggaacacagattgtcgtcaaaaaagacaatacagacatcaaatcagtagacgatttaaaaggcaagacagtcgcagccgttctcggttcaaaccacgcgaaaaaccttgaaagcaaagatcctgataaaaaaatcaatatcaaaacgtacgaaacacaagagggtacgctgaaggatgttgcgtacggccgtgtagacgcttatgtcaacagccgaactgtattgatcgcgcaaatcaagaagaccggtttgccattaaagcttgcaggagatccgattgtttacgaacaggttgcattcccatttgccaaggacgatgcgcacgacaagctccgcaaaaaagtcaataaggccctagatgaattgcgtaaagacggaacactgaaaaaactctctgaaaaatactttaatgaagatatcacagtagaacagaagcattaa

SEQ ID NO: 80 - DNA, 498 bp, Bacillus subtilis
atgaagccacgataccgccttgcagttgaacgtgatgccgaacagcttctcgagctgacattgcgggcttatgaaccgattcgaaagctcggcattcgttttgctgctgctcatgcggatttggatttggtgctgaaaaatattcgggaaaatgcttgctacgtcatggaagaagacgggcggatcatcgcgaccatcaccttgagaatgccttggggaaaacagccgggaccgtatggcgttccgcatatctggtggtttgctgtggaccccgacaccggtaaaaaaggaatcggtacaaagctgcttcaatggctggaggaaacaatccttcgcgatacgttaaaggttccgtttgtttcactcggaacagcggataagcatccgtggctgattgagatgtacgaacgaaaaggatatgtccgctcaggtgaacaagaccttggaaaagggcatatcacagtctatatgaaaaaacaattgggacatgatctataa

SEQ ID NO: 81 - DNA, 1326 bp, Bacillus subtilis
atgacaagcaaaaagaaacaaatcaaattaggggtatttttagcaggtacaggccatcatgttgcgtcttggcggcacccggacgcgccgtcagatgcgagcatgaatttggattattttaaagagcttgcgaaaacagcggagcgaggcaagctggatatgctgtttttagcggacagcctttcaattgacagcaaatcacatccaaatgtattaacaaggtttgagccattcaccctgctctctgctttggcgcaggtcacatcaaaaatcggactgacagcaacagcctccactacatacagcgagccattccatattgccagacagtttgcgtcattggatcatctgtccaatggccgtgccggatggaacgtcgtcacttcatctattgaatcaacagcgctgaatttcagcggtgaaaagcaccttgaacaccatttgcgctatcagcgggcagaggaatttgtcgagattgtaaaggggctttgggattcatgggaagaggacgcctttatccgtaataaagaaacgggtgaattctttgacaaagaaaaaatgcatgagctgaaccacaaaggagaatatttctcggttcgcggacctctaaacgtttcaagaaccccgcagggccagccggtcattatccaggcaggatcatcaggagacggaaaagcgctggctgccaaaacagccgaagtgatcttcacagcacaaaaccacctggaatcagctcaagaattttatcaatccattaaagaacaggctgcggaattcggacgtgatccagaaaaaattgccattatgccgggtattttcccaatcattgccgatacagaagaagcagcgcaagccaaatacaaggagctccaagatctgattatcccatctgtcggtctgcaaattctccaaaattacttaggcggaattgatttgtcggcatatccgcttgatgggccgctgccgaagcttgacgccgaagcttccaatgcggtgaagagccgcttcaagcttgttcaggagatggctgaacgtgacaatatgacgatacgagagctttacaaatacgttgcaggctccagaggccaccatatcttcgtcggcacgccggagcagctcgccgacaagatgcaggaatgggtggatacgaaagcgtgtgacgggtttaacatcatgcctccgcttcttccagaaggaattgaagtgtttgttgatcaagtggttccgattttacaggagcgcggcgtgttcagaaaagaatatgaaggcacaacattacgagagcacttcggtttggaaaagccggtaaaccgctatgcaaagtaa